(19) United States
(12) Patent Application Publication
(10) Pub. No.: US 2006/0116393 A1
(43) Pub. Date: Jun. 1, 2006

(54) Substituted pyrido [2,1-a] isoquinoline derivatives

(76) Inventors: Markus Boehringer, Moehlin (CH); Daniel Hunziker, Moehlin (CH); Bernd Kuhn, Liestal (CH); Bernd Michael Loeffler, Zug (CH); Fabienne Ricklin, Hombourg (FR); Hans Peter Wessel, Schliengen (DE)

Correspondence Address: HOFFMANN-LA ROCHE INC.;PATENT LAW DEPARTMENT, 340 KINGSLAND STREET, NUTLEY, NJ, 07110, US

(21) Appl. No.: 11/288,648

(22) Filed: Nov. 28, 2005

(30) Foreign Application Priority Data
Nov. 30, 2004 (EP) 04106191.2

(51) Int. Cl. A61K 31/4745; C07D 451/02

(52) U.S. Cl. 514/294; 546/94

(57) ABSTRACT

The present invention relates to compounds of formula (I) 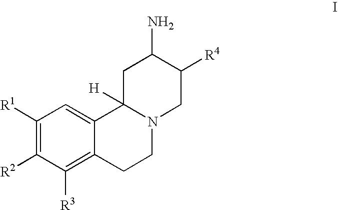
wherein R¹ to R⁴ are as defined in the description and claims, and pharmaceutically acceptable salts thereof. The compounds are useful for the treatment and/or prophylaxis of diseases which are associated with DPP-IV, such as diabetes, particularly non-insulin dependent diabetes mellitus, and impaired glucose tolerance.

### FIELD OF THE INVENTION

The present invention is directed to novel pyrido[2,1-a]isoquinoline derivatives, their manufacture and their use as medicaments.

In one embodiment, the invention is directed to compounds of the general formula 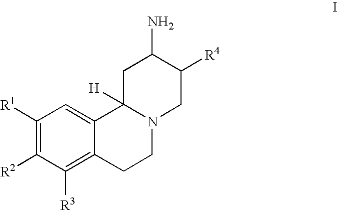
and pharmaceutically acceptable salts thereof.

All documents cited or relied upon below are expressly incorporated herein by reference.

### BACKGROUND OF THE INVENTION

The enzyme dipeptidyl peptidase IV (EC.3.4.14.5, abbreviated in the following as DPP-IV) is involved in the regulation of the activities of several hormones. In particular DPP-IV efficiently and rapidly degrades glucagon like peptide 1 (GLP-1), which is one of the most potent stimulator of insulin production and secretion. Inhibiting DPP-IV would potentiate the effect of endogenous GLP-1, and lead to higher plasma insulin concentrations. In patients suffering from impaired glucose tolerance and type 2 diabetes mellitus, higher plasma insulin concentration would moderate the dangerous hyperglycaemia and accordingly reduce the risk of tissue damage. Consequently, DPP-IV inhibitors have been suggested as drug candidates for the treatment of impaired glucose tolerance and type 2 diabetes mellitus (e.g. Villhauer, WO98/19998). Other related state of the art can be found in WO 99/38501, DE 19616486, DE 19834591, WO 01/40180, WO 01/55105, U.S. Pat. No. 6,110,949, WO 00/34241 and U.S. Pat. No. 6,011,155.

Furthermore, DPP IV contributes to the generation and modulation of a T cell immune response. DPP IV (also known as CD26) has an essential role in immune regulation as a T cell activation molecule and a regulator of chemokine function thus suggesting a role for DPP-IV in the pathophysiology of immune-mediated disorders as well as autoimmune diseases (Hosano O. et al., Modern Rheumatology 2003, 13(3), 199-204). Abnormal expression of DPP-IV is found in the case of autoimmune diseases, HIV-related diseases and cancer. Natural substrates for DPP-IV are involved in immunomodulation, psycho/neuronal modulation and physiological processes in general (Boonacker E.; Van Noorden C. J. F, European Journal of Cell Biology 2003, 82(2), 53-73). Furthermore, it has been shown that there is a correlation between DPP-IV and the key nuclear protein topoisomerase alpha (Aytac U., Dang, N. H., Current Drug Targets: Immune, Endocrine and Metabolic Disorders 2004, 4(1), 11-18). Thus, DPP-IV inhibitors may be useful as medicaments for the treatment of various diseases in which DPP-IV is involved.

### SUMMARY OF THE INVENTION

In one embodiment of the present invention, provided is a compound of the formula (I):

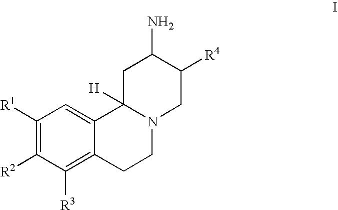

wherein

R¹ is selected from hydrogen or methoxy;

R² is selected from the group consisting of

hydroxy,

lower alkoxy, provided that R² is not methoxy in case R¹ is methoxy,

lower alkoxy mono- or disubstituted by
hydroxy, lower alkoxy, benzyloxy,
amino, alkylamino, dialkylamino, cyano,
unsubstituted phenyl, phenyl substituted by one to three groups selected from lower alkyl, lower alkoxy, halogen and lower halogenalkyl, or
tetrazolyl,

—O—(CH₂)ₘ—C(O)—NR⁸R⁹, wherein m is 1 or 2 and wherein R⁸ and R⁹ are independently selected from hydrogen, lower alkyl or tetrazolyl, or R⁸ and R⁹ together with the nitrogen atom to which they are attached form a 5- or 6-membered heterocycle which may contain an additional heteroatom selected from N, O or S, and which may be substituted by lower alkyl,

—O—(CH₂)ₙ—COOR¹⁰, wherein n is 1 or 2 and R¹⁰ is hydrogen or lower alkyl,

—O—(CH₂)ₚ—NH—C(O)—OR¹¹, wherein p is 1 or 2 and wherein R¹¹ is lower alkyl,

—O—SO₂—R ², wherein R¹² is lower alkyl,

—NR¹³R¹⁴, wherein R¹³ is hydrogen or lower alkyl and R¹⁴ is lower alkyl or benzyl, and

—NH—CO—(CH₂)_{q}—R¹⁵, wherein q is 1 or 2 and wherein R¹⁵ is lower alkyl or tetrazolyl;

R³ is selected from the group consisting of

hydrogen,

hydroxy,

lower alkoxy,

lower alkoxy mono- or disubstituted by
hydroxy, alkoxy, benzyloxy,
amino, alkylamino, dialkylamino, cyano,
unsubstituted phenyl, phenyl substituted one to three groups selected from lower alkyl, lower alkoxy, halogen and lower halogenalkyl, or
tetrazolyl,

—O—(CH₂)ₘ—C(O)—NR⁸R⁹, wherein m is 1 or 2 and wherein R⁸ and R⁹ are independently selected from hydrogen, lower alkyl or tetrazolyl, or R⁸ and R⁹ together with the nitrogen atom to which they are attached form a 5- or 6-membered heterocycle which may contain an additional heteroatom selected from N, O or S, and which may be substituted by lower alkyl,

—O—(CH₂)ₙ—COOR¹⁰, wherein n is 1 or 2 and R¹⁰ is hydrogen or lower alkyl,

—O—(CH₂)ₚ—NH—C(O)—OR¹¹, wherein p is 1 or 2 and wherein R¹¹ is lower alkyl,

—O—SO₂—R¹², wherein R¹² is lower alkyl

—NR¹³R¹⁴, wherein R¹³ is hydrogen or lower alkyl and R¹⁴ is lower alkyl or benzyl, and

—NH—CO—(CH₂)_{q}—R¹⁵, wherein q is 1 or 2 and wherein R¹⁵ is lower alkyl or tetrazolyl;

R⁴ is

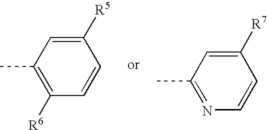

wherein

R⁵ is selected from the group consisting of lower alkyl, lower hydroxyalkyl, lower halogenalkyl, halogen and cycloalkyl; or

R⁵ can also be hydrogen in case R² is selected from the group consisting of —(CH₂)ₘ—C(O)—NR⁸R⁹, —O—(CH₂)ₚ—NH—C(O)—OR¹¹, —O—SO₂—R¹², —NR¹³R¹⁴, —NH—CO—(CH₂)_{q}—R¹⁵ and lower alkoxy which is mono- or disubstituted by a group selected from hydroxy, benzyloxy, amino, alkylamino, dialkylamino or cyano;

R⁶ is selected from the group consisting of hydrogen, lower alkyl, lower alkoxy, lower hydroxyalkyl, lower halogenalkyl, halogen and cycloalkyl;

R⁷ is selected from the group consisting of lower alkyl, cycloalkyl, lower hydroxyalkyl, halogen and lower halogenalkyl;

and pharmaceutically acceptable salts thereof.

In another embodiment of the present invention, provided is a process for the manufacture of the compound according to formula I, which process comprises the steps of: converting a compound of the formula

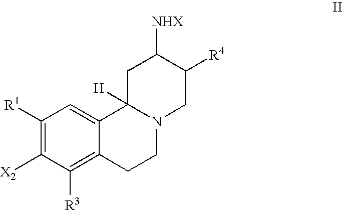

wherein X is hydrogen or tert-butoxycarbonyl, X₂ is —OH or —NH₂, R¹ and R⁴ are as defined above and R³ is hydrogen, by side chain transformation into a compound of the formula

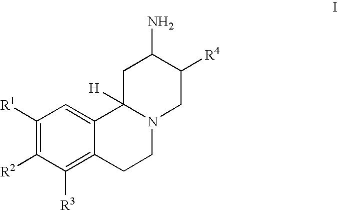

wherein R¹, R² and R⁴ are defined above and R³ is hydrogen, or alternatively, converting a compound of the formula

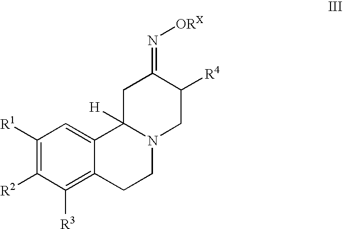

wherein R^{x} is hydrogen or benzyl and R¹ to R⁴ are as defined above, by catalytic hydrogen reduction into a compound of the formula

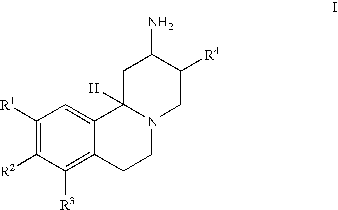

wherein R¹ to R⁴ are defined as above,

and optionally converting the compound of formula I into a pharmaceutically acceptable salt.

In a further embodiment of the present invention, provided is a pharmaceutical composition, comprising a therapeutically effective amount of a compound according to claim 1 and a pharmaceutically acceptable carrier and/or adjuvant.

In a yet another embodiment of the present invention, provided is a method for the treatment and/or prophylaxis of diseases which are associated with DPP-IV comprising the step of administering a therapeutically effective amount of a compound according to claim 1 to a human being or animal in need thereof.

### DETAILED DESCRIPTION

The invention provides for novel DPP-IV inhibitors that very efficiently lower plasma glucose levels. Consequently, the compounds of the present invention are useful for the treatment and/or prophylaxis of diabetes, particularly non-insulin dependent diabetes mellitus, and/or impaired glucose tolerance, as well as other conditions wherein the amplification of action of a peptide normally inactivated by DPP-IV gives a therapeutic benefit. In addition, the compounds of the present invention can also be used in the treatment and/or prophylaxis of obesity, metabolic syndrome, β-cell protection, autoimmune diseases such as inflammatory bowel disease, encephalitis periaxialis scleroticans and rheumatoid arthritis, Colitis Ulcerosa, Morbus Crohn, psoriasis, lichen planus and/or benign prostate hypertrophy. The compounds may also be useful for the prevention of AIDS (acquired immunodeficiency syndrome) or for preventing metastasis, particularly preventing metastasis of breast and prostate cancer to lung. Furthermore, the compounds of the present invention can be used as diuretic agents and for the treatment and/or prophylaxis of hypertension.

The compounds of the present invention exhibit improved therapeutic and pharmacological properties compared to other DPP-IV inhibitors known in the art, such as e.g. in context of pharmacokinetics and bioavailability.

Unless otherwise indicated, the following definitions are set forth to illustrate and define the meaning and scope of the various terms used to describe the invention herein.

In this specification the term “lower” is used to mean a group consisting of one to six, preferably of one to four carbon atom(s).

The term “halogen” refers to fluorine, chlorine, bromine and iodine, with fluorine and chlorine being preferred. Most preferred halogen is chlorine.

The term “alkyl”, alone or in combination with other groups, refers to a branched or straight-chain monovalent saturated aliphatic hydrocarbon radical of one to twenty carbon atoms, preferably one to sixteen carbon atoms, more preferably one to ten carbon atoms.

The term “lower alkyl”, alone or in combination with other groups, refers to a branched or straight-chain monovalent alkyl radical of one to six carbon atoms, preferably one to four carbon atoms. This term is further exemplified by radicals such as methyl, ethyl, n-propyl, isopropyl, n-butyl, s-butyl, isobutyl, t-butyl, n-pentyl, 3-methylbutyl, n-hexyl, 2-ethylbutyl and the like. Preferable lower alkyl residues are methyl and ethyl, with methyl being especially preferred.

The term “lower halogenalkyl” refers to a lower alkyl group wherein at least one of the hydrogens of the lower alkyl group is replaced by a halogen atom, preferably fluoro or chloro, most preferably fluoro. Among the preferred lower halogenalkyl groups are trifluoromethyl, difluoromethyl, fluoromethyl and chloromethyl, with fluoromethyl and trifluoromethyl being especially preferred.

The term “alkoxy” refers to the group R′—O—, wherein R′ is alkyl. The term “lower-alkoxy” refers to the group R′—O—, wherein R′ is lower alkyl. Examples of lower alkoxy groups are e.g. methoxy, ethoxy, propoxy, isopropoxy, butoxy, isobutoxy and hexyloxy, with methoxy being especially preferred.

The term “lower hydroxyalkyl” refers to a lower alkyl group wherein at least one of the hydrogens of the lower alkyl group is replaced by a hydroxy group. Among the preferred lower hydroxyalkyl groups are hydroxymethyl, 2-hydroxyethyl, 2,3-dihydroxypropyl, and 1-hydroxymethyl-2-hydroxyethyl.

The term “cycloalkyl” refers to a monovalent carbocyclic radical of three to six, preferably three to five carbon atoms. This term is further exemplified by radicals such as cyclopropyl, cyclobutyl, cyclopentyl and cyclohexyl, with cyclopropyl being preferred.

The term “R⁸ and R⁹ together with the nitrogen atom to which they are attached form a 5- or 6-membered heterocycle which may contain an additional heteroatom selected from N, O or S” means that R⁸ and R⁹ together with the nitrogen atom form a ring such as pyrrolidinyl, piperidyl, imidazolidinyl, pyrazolidinyl, morpholinyl, piperazinyl or thiomorpholinyl, with morpholinyl and piperazinyl being especially preferred.

The term “pharmaceutically acceptable salts” refers to salts of the compounds of formula (I) with inorganic or organic acids such as hydrochloric acid, hydrobromic acid, nitric acid, sulphuric acid, phosphoric acid, citric acid, formic acid, maleic acid, acetic acid, fumaric acid, succinic acid, tartaric acid, methanesulphonic acid, salicylic acid, p-toluenesulphonic acid and the like, which are non toxic to living organisms. Preferred salts with acids are formates, maleates, citrates, hydrochlorides, hydrobromides and methanesulfonic acid salts, with hydrochlorides being especially preferred.

In detail, the present invention relates to compounds of the general formula

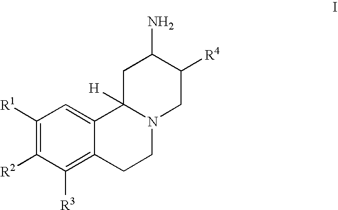

wherein

R¹ is selected from hydrogen or methoxy;

R² is selected from the group consisting of

hydroxy,

lower alkoxy, provided that R² is not methoxy in case R¹ is methoxy,

lower alkoxy mono- or disubstituted by hydroxy, lower alkoxy, benzyloxy,

amino, alkylamino, dialkylamino, cyano,

unsubstituted phenyl, phenyl substituted by one to three groups selected from lower alkyl, lower alkoxy, halogen or lower halogenalkyl, tetrazolyl,

—O—(CH₂)ₘ—C(O)—NR⁸R⁹, wherein m is 1 or 2 and wherein R⁸ and R⁹ are independently selected from hydrogen, lower allyl or tetrazolyl, or R⁸ and R⁹ together with the nitrogen atom to which they are attached form a 5- or 6-membered heterocycle which may contain an additional heteroatom selected from N, O or S, and which may be substituted by lower alkyl,

—O—(CH₂)ₙ—COOR¹⁰, wherein n is 1 or 2 and R¹⁰ is hydrogen or lower alkyl,

—O—(CH₂)ₚ—NH—C(O)—OR¹¹, wherein p is 1 or 2 and wherein R¹¹ is lower alkyl,

—O—SO₂—R ², wherein R¹² is lower alkyl,

—NR¹³R¹⁴, wherein R¹³ is hydrogen or lower alkyl and R¹⁴ is lower alkyl or benzyl, and

—NH—CO—(CH₂)_{q}—R ⁵, wherein q is 1 or 2 and wherein R¹⁵ is lower alkyl or tetrazolyl;

R³ is selected from the group consisting of

hydrogen,

hydroxy,

lower alkoxy,

lower alkoxy mono- or disubstituted by

hydroxy, alkoxy, benzyloxy,

amino, alkylamino, dialkylamino, cyano,

unsubstituted phenyl, phenyl substituted one to three groups selected from lower alkyl, lower alkoxy, halogen or lower halogenalkyl,

tetrazolyl, and

—O—(CH₂)ₘ—C(O)—NR⁸R⁹, wherein m is 1 or 2 and wherein R⁸ and R⁹ are independently selected from hydrogen, lower alkyl or tetrazolyl, or R⁸ and R⁹ together with the nitrogen atom to which they are attached form a 5- or 6-membered heterocycle which may contain an additional heteroatom selected from N, O or S, and which may be substituted by lower alkyl,

—O—(CH₂)ₙ—COOR¹⁰, wherein n is 1 or 2 and R¹⁰ is hydrogen or lower alkyl,

—O—(CH₂)ₚ—NH—C(O)—OR¹¹, wherein p is 1 or 2 and wherein R¹¹ is lower alkyl,

—O—SO₂—R¹², wherein R¹² is lower alkyl,

—NR¹³R¹⁴, wherein R¹³ is hydrogen or lower alkyl and R¹⁴ is lower alkyl or benzyl, and

—NH—CO—(CH₂)_{q}—R¹⁵, wherein q is 1 or 2 and wherein R¹⁵ is lower alkyl or tetrazolyl;

R⁴ is

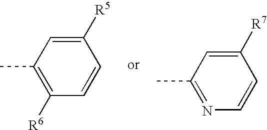

wherein

R⁵ is selected from the group consisting of lower alkyl, lower hydroxyalkyl, lower halogenalkyl, halogen and cycloalkyl; or

R⁵ can also be hydrogen in case R² is selected from the group consisting of

—(CH₂)ₘ—C(O)—NR⁸R⁹, —O—(CH₂)ₚ—NH—C(O)—OR¹¹, —O—SO₂—R , —NR¹³R¹⁴,

—NH—CO—(CH₂)_{q}—R¹⁵ and lower alkoxy which is mono- or disubstituted by a group selected from hydroxy, benzyloxy, amino, alkylamino, dialkylamino or cyano;

R⁶ is selected from the group consisting of hydrogen, lower alkyl, lower alkoxy, lower hydroxyalkyl, lower halogenalkyl, halogen and cycloalkyl;

R⁷ is selected from the group consisting of lower alkyl, cycloalkyl, lower hydroxyalkyl, halogen and lower halogenalkyl;

and pharmaceutically acceptable salts thereof.

The present invention further includes all specific stereoisomers and enantiomers of the compounds of formula I.

In one embodiment, the invention relates to compounds of formula I as defined above, wherein R⁴ is phenyl and R² is selected from the group consisting of —(CH₂)ₘC(O)—NR⁸R⁹, —O—(CH₂)ₚ—NH—C(O)—OR¹¹, —O—SO₂—R¹², —NR¹³R¹⁴, —NH—CO—(CH₂)_{q}—R¹⁵ and lower alkoxy which is mono- or disubstituted by a group selected from hydroxy, benzyloxy, amino, alkylamino, dialkylamino or cyano, with those compounds wherein R⁴ is phenyl and R² is —(CH₂)ₘ—C(O)—NR⁸R⁹ being especially preferred.

Preferred compounds of formula I as defined above are those compounds, wherein

R⁴ is

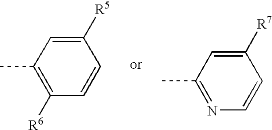

and wherein

R⁵ is selected from the group consisting of lower alkyl, lower hydroxyalkyl, lower halogenalkyl, halogen and cycloalkyl;

R⁶ is selected from the group consisting of hydrogen, lower alkyl, lower alkoxy, lower hydroxyalkyl, lower halogenalkyl, halogen and cycloalkyl; and

R⁷ is selected from the group consisting of lower alkyl, cycloalkyl, lower hydroxyalkyl, halogen and lower halogenalkyl.

Further preferred are compounds of formula I of the present invention, wherein R² is selected from the group consisting of

hydroxy,

lower alkoxy, provided that R² is not methoxy in case R¹ is methoxy,

lower alkoxy mono- or disubstituted by

hydroxy, lower alkoxy, benzyloxy,

amino, alkylamino, dialkylamino, cyano,

unsubstituted phenyl, phenyl substituted by one to three groups selected from lower alkyl, lower alkoxy, halogen or lower halogenalkyl,

tetrazolyl,

—O—(CH₂)ₘ—C(O)—NR⁸R⁹, wherein m is 1 or 2 and wherein R⁸ and R⁹ are independently selected from hydrogen, lower alkyl or tetrazolyl, or R⁸ and R⁹ together with the nitrogen atom to which they are attached form a 5- or 6-membered heterocycle which may contain an additional heteroatom selected from N, O or S, and which may be substituted by lower alkyl,

—O—(CH₂)ₙ—COOR¹⁰, wherein n is 1 or 2 and R¹⁰ is hydrogen or lower alkyl,

—O—(CH₂)ₚ—NH—C(O)—OR¹¹, wherein p is 1 or 2 and wherein R¹¹ is lower alkyl,

—O—SO₂—R¹², wherein R¹² is lower alkyl,

—NR¹³R¹⁴, wherein R¹³ is hydrogen or lower alkyl and R¹⁴ is lower alkyl or benzyl, and

—NH—CO—(CH₂)_{q}—R¹⁵, wherein q is 1 or 2 and wherein R¹⁵ is lower alkyl or tetrazolyl;

and R³ is hydrogen, hydroxy or lower alkoxy.

More preferred are compounds of formula I of the present invention, wherein R² is selected from the group consisting of

hydroxy,

lower alkoxy mono- or disubstituted by

hydroxy, benzyloxy, amino, cyano, phenyl or tetrazolyl,

—O—(CH₂)ₘ—C(O)—NR⁸R⁹, wherein m is 1 or 2 and wherein R⁸ and R⁹ are independently selected from hydrogen, lower alkyl or tetrazolyl, or R⁸ and R⁹ together with the nitrogen atom to which they are attached form a 5- or 6-membered heterocycle which may contain an additional heteroatom selected from N, O or S, and which may be substituted by lower alkyl,

—O—(CH₂)ₙ—COOR¹⁰, wherein n is 1 or 2 and R¹⁰ is hydrogen or lower alkyl,

—O—(CH₂)ₚ—NH—C(O)—OR¹¹, wherein p is 1 or 2 and wherein R¹¹ is lower alkyl,

—O—SO₂—R¹², wherein R¹² is lower alkyl,

—NR¹³R¹⁴, wherein R¹³ is hydrogen or lower alkyl and R¹⁴ is lower alkyl or benzyl, and

—NH—CO—(CH₂)_{q}—R¹⁵, wherein q is 1 or 2 and wherein R¹⁵ is lower alkyl or tetrazolyl.

Within this group, compounds of formula I are preferred wherein R² is hydroxy or lower alkoxy mono- or disubstituted by hydroxy, benzyloxy, amino, cyano, phenyl or tetrazolyl.

Further preferred are compounds of formula I, wherein R² is

—O—(CH₂)ₘ—C(O)—NR⁸R⁹, wherein m is 1 or 2 and wherein R⁸ and R⁹ are independently selected from hydrogen, lower alkyl or tetrazolyl, or R⁸ and R⁹ together with the nitrogen atom to which they are attached form a 5- or 6-membered heterocycle which may contain an additional heteroatom selected from N, O or S, and which may be substituted by lower alkyl, with those compounds of formula I, wherein R² is —O—(CH₂)ₘ—C(O)—NR⁸R⁹, wherein m is 1 or 2 and wherein R⁸ and R⁹ are independently selected from hydrogen, lower alkyl or tetrazolyl. Also preferred are compounds of formula I, wherein R² is —O—(CH₂)ₙ—COOR¹⁰, wherein n is 1 or 2 and R¹⁰ is hydrogen or lower alkyl.

Furthermore, compounds of formula I are preferred, wherein R² is —O—SO₂—R¹², wherein R¹² is lower alkyl.

Further preferred are compounds of formula I, wherein R² is —NH—CO—(CH₂)_{q}—R¹⁵, wherein q is 1 or 2 and wherein R¹⁵ is lower alkyl of tetrazolyl.

Especially preferred are compounds of formula I, wherein R² is defined as described above and R³ is hydrogen.

Furthermore, compounds of formula I of the present invention are preferred, wherein R³ is selected from the group consisting of

hydroxy,

lower alkoxy,

lower alkoxy mono- or disubstituted by

hydroxy, alkoxy, benzyloxy,

amino, alkylamino, dialllylamino, cyano,

unsubstituted phenyl, phenyl substituted one to three groups selected from lower alkyl, lower

alkoxy, halogen or lower halogenalkyl,

tetrazolyl, and

—O—(CH₂)ₘ—C(O)—NR⁸R⁹, wherein m is 1 or 2 and wherein R⁸ and R⁹ are independently selected from hydrogen, lower alkyl or tetrazolyl, or R⁸ and R⁹ together with the nitrogen atom to which they are attached form a 5- or 6-membered heterocycle which may contain an additional heteroatom selected from N, O or S, and which may be substituted by lower alkyl, and R² is hydroxy or lower alkoxy.

Within this group, compounds of formula I are preferred, wherein R³ is hydroxy or lower alkoxy mono- or disubstituted by hydroxy, alkoxy, benzyloxy or phenyl.

Also preferred are compounds of formula I, wherein R³ is —O—(CH₂)ₘ—C(O)—NR⁸R⁹, wherein m is 1 or 2 and wherein R⁸ and R⁹ are independently selected from hydrogen, lower alkyl or tetrazolyl, or R⁸ and R⁹ together with the nitrogen atom to which they are attached form a 5- or 6-membered heterocycle which may contain an additional heteroatom selected from N, O or S, and which may be substituted by lower alkyl.

Furthermore, compounds of formula I of the present invention are preferred, wherein R³ is selected from the group consisting of

hydroxy,

lower alkoxy,

lower alkoxy mono- or disubstituted by

hydroxy, alkoxy, benzyloxy,

amino, alkylamino, dialkylamino, cyano,

unsubstituted phenyl, phenyl substituted one to three groups selected from lower alkyl, lower alkoxy, halogen or lower halogenalkyl,

tetrazolyl, and

—O—(CH₂)ₘ—C(O)—NR⁸R⁹, wherein m is 1 or 2 and wherein R⁸ and R⁹ are independently selected from hydrogen, lower alkyl or tetrazolyl, or R⁸ and R⁹ together with the nitrogen atom to which they are attached form a 5- or 6-membered heterocycle which may contain an additional heteroatom selected from N, O or S, and which may be substituted by lower alkyl, and R² is methoxy.

Furthermore, compounds of formula I of the present invention are preferred, wherein R⁴ is

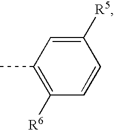

R⁵ is selected from the group consisting of lower alkyl, lower hydroxyalkyl, lower halogenalkyl, halogen and cycloalkyl; and

R⁶ is selected from the group consisting of hydrogen, lower alkyl, lower alkoxy, lower hydroxyalkyl, lower halogenalkyl, halogen and cycloalkyl, with those compounds, wherein R⁵ is lower alkyl or lower halogenalkyl, and R⁶ is hydrogen or lower alkyl, being especially preferred.

Also preferred are compounds of formula I according to the present invention, wherein R⁴ is

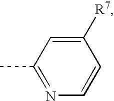

and R⁷ is selected from the group consisting of lower alkyl, cycloalkyl, lower hydroxyalkyl, halogen and lower halogenalkyl, with those compounds, wherein R⁷ is lower alkyl, being especially preferred.

Preferred compounds of the general formula I are those selected from the group consisting of:
(2S,3S,11bS)- and (2R,3R,11bR)-9-(2-amino-ethoxy)-3-(2,5-dimethyl-phenyl)-10-methoxy-1,3,4,6,7,11b-hexahydro-2H-pyrido[2,1-a]isoquinolin-2-ylamine,
(2S,3S,11bS)- and (2R,3R,11bR)-2-amino-3-(2,5-dimethyl-phenyl)-10-methoxy-1,3,4,6,7,11b-hexahydro-2H-pyrido[2,1-a]isoquinolin-9-ol,
(2S,3S,11bS)- and (2R,3R,11bR)-{2-[2-amino-3-(2,5-dimethyl-phenyl)-10-methoxy-1,3,4,6,7,11b-hexahydro-2H-pyrido[2,1-a]isoquinolin-9-yloxy]-ethyl}-carbamic acid tert-butyl ester,
(2S,3S,11bS)- and (2R,3R,11bR)-3-(2,5-dimethyl-phenyl)-10-methoxy-9-[2-(1H-tetrazol-5-yl)-ethoxy]-1,3,4,6,7,11b-hexahydro-2H-pyrido[2,1-a]isoquinolin-2-ylamine hydrochloride,
(2S,3S,11bS)- and (2R,3R,11bR)-3-[2-amino-3-(2,5-dimethyl-phenyl)-10-methoxy-1,3,4,6,7,11b-hexahydro-2H-pyrido[2,1-a]isoquinolin-9-yloxy]-propionitrile,
(2S,3S,11bS)- and (2R,3R,11bR)-methanesulfonic acid 2-amino-3-(2,5-dimethyl-phenyl)-10-methoxy-1,3,4,6,7,11b-hexahydro-2H-pyrido[2,1-a]isoquinolin-9-yl ester hydrochloride,
(2S,3S,11bS)- and (2R,3R,11bR)-2-[2-amino-3-(2,5-dimethyl-phenyl)-10-methoxy-1,3,4,6,7,11b-hexahydro-2H-pyrido[2,1-a]isoquinolin-9-yloxy]-ethanol hydrochloride
(2S,3S,11bS)- and (2R,3R,11bR)-[2-amino-3-(2,5-dimethyl-phenyl)-10-methoxy-1,3,4,6,7,11b-hexahydro-2H-pyrido[2,1-a]isoquinolin-9-yloxy]-acetic acid hydrochloride,
(2S,3S,11bS)- and (2R,3R,11bR)-2-[2-amino-3-(2,5-dimethyl-phenyl)-10-methoxy-1,3,4,6,7,11b-hexahydro-2H-pyrido[2,1-a]isoquinolin-9-yloxy]-N-(1H-tetrazol-5-yl)-acetamide hydrochloride,
(2S,3S,11bS)- and (2R,3R,11bR)-2-[2-amino-3-(2,5-dimethyl-phenyl)-10-methoxy-1,3,4,6,7,11b-hexahydro-2H-pyrido[2,1-a]isoquinolin-9-yloxy]-acetamide hydrochloride,
(2S,3S,11bS)- and (2R,3R,11bR)-2-[2-amino-3-(2,5-dimethyl-phenyl)-10-methoxy-1,3,4,6,7,11b-hexahydro-2H-pyrido[2,1-a]isoquinolin-9-yloxy]-N-methyl-acetamide hydrochloride,
(2S,3S,11bS)- and (2R,3R,11bR)-N-[2-amino-10-methoxy-3-(4-methyl-pyridin-2-yl)-1,3,4,6,7,11b-hexahydro-2H-pyrido[2,1-a]isoquinolin-9-yl]-2-(1H-tetrazol-5-yl)-acetamide hydrochloride,
(2S,3S,11bS)- and (2R,3R,11bR)-N9-benzyl-10-methoxy-3-(4-methyl-pyridin-2-yl)-1,3,4,6,7,11b-hexahydro-2H-pyrido[2,1-a]isoquinolin-2,9-diamine hydrochloride,
(2S,3R,11RS)- and (2R,3S,11bS)-N9-benzyl-10-methoxy-3-(4-methyl-pyridin-2-yl)-1,3,4,6,7,11b-hexahydro-2H-pyrido[2,1-a]isoquinolin-2,9-diamine,
(2S,3S,11bS)- and (2R,3R,11bR)-10-methoxy-9-methylamino-3-(4-methyl-pyridin-2-yl)-1,3,4,6,7,11b-hexahydro-2H-pyrido[2,1-a]isoquinoline-2,9-diamine hydrochloride,
(2S,3S,11bS)- and (2R,3R,11bR)-[2-amino-10-methoxy-3-(4-methyl-pyridin-2-yl)-1,3,4,6,7,11b-hexahydro-2H-pyrido[2,1-a]isoquinolin-9-yloxy]-acetic acid hydrochloride,
(2S,3S,11bS)- and (2R,3R,11bR)-9-benzyloxy-10-methoxy-3-(4-methyl-pyridin-2-yl)-1,3,4,6,7,11b-hexahydro-2H-pyrido[2,1-a]isoquinolin-2-ylamine,
(2S,3S,11bS)- and (2R,3R,11bR)-2-[2-amino-10-methoxy-3-(4-methyl-pyridin-2-yl)-1,3,4,6,7,11b-hexahydro-2H-pyrido[2,1-a]isoquinolin-9-yloxy]-N-(2H-tetrazol-5-yl)-acetamide hydrochloride,
(2S,3S,11bS)- and (2R,3R,11bR)-methanesulfonic acid 2-amino-10-methoxy-3-phenyl-1,3,4,6,7,11b-hexahydro-2H-pyrido[2,1-a]isoquinolin-9-yl ester,
(2S,3S,11bS)- and (2R,3R,11bR)-2-(2-amino-10-methoxy-3-phenyl-1,3,4,6,7,11b-hexahydro-2H-pyrido[2,1-a]isoquinolin-9-yloxy)-ethanol,
(2S,3S,11bS)- and (2R,3R,11bR)-9-(2-benzyloxy-ethoxy)-10-methoxy-3-phenyl-1,3,4,6,7,11b-hexahydro-2H-pyrido[2,1-a]isoquinolin-2-ylamine,
(2S,3S,11bS)- and (2R,3R,11bR)-9-(2-amino-ethoxy)-10-methoxy-3-phenyl-1,3,4,6,7,1 1b-hexahydro-2H-pyrido[2,1-a]isoquinolin-2-ylamine,
(2S,3S,11bS)- and (2R,3R,11bR)-2-(2-amino-10-methoxy-3-phenyl-1,3,4,6,7,11b-hexahydro-2H-pyrido[2,1-a]isoquinolin-9-yloxy) -N-methyl-acetamide,
(2S,3S,11bS)- and (2R,3R,11bR)-2-(2-amino-10-methoxy-3-phenyl-1,3,4,6,7,11b-hexahydro-2H-pyrido[2,1-a]isoquinolin-9-yloxy) -N,N-dimethyl-acetamide,
(2S,3S,11bS)- and (2R,3R,11bR)-2-(2-amino-10-methoxy-3-phenyl-1,3,4,6,7,11b-hexahydro-2H-pyrido[2,1-a]isoquinolin-9-yloxy)-acetamide,
(2S,3S,11bS)- and (2R,3R,11bR)-9-benzyloxy-10-methoxy-3-m-tolyl-1,3,4,6,7,11b-hexahydro-2H-pyrido[2,1-a]isoquinolin-2-ylamine,
(2S,3S,11bS)- and (2R,3R,11bR)-2-amino-10-methoxy-3-m-tolyl-1,3,4,6,7,11b-hexahydro-2H-pyrido[2,1-a]isoquinolin-9-ol,
(2S,3S,11bS)- and (2R,3R,11bR)-9-(2-benzyloxy-ethoxy)-10-methoxy-3-m-tolyl-1,3,4,6,7,11b-hexahydro-2H-pyrido[2,1-a]isoquinolin-2-ylamine,
(2S,3S,11bS)- and (2R,3R,11bR)-2-(2-amino-10-methoxy-3-m-tolyl-1,3,4,6,7,11b-hexahydro-2H-pyrido[2,1-a]isoquinolin-9-yloxy)-ethanol,
(2R,3S,11bS)- and (2S,3R,11bR)-9-benzyloxy-10-methoxy-3-m-tolyl-1,3,4,6,7,11b-hexahydro-2H-pyrido[2,1-a) isoquinolin-2-ylamine,
(2R,3S,11bS)- and (2S,3R,11bR)-2-amino-10-methoxy-3-m-tolyl-1,3,4,6,7,11b-hexahydro-2H-pyrido[2,1-a]isoquinolin-9-ol,
(2S,3S,11bS)- and (2R,3R,11bR)-2-(2-amino-10-methoxy-3-m-tolyl-1,3,4,6,7,11b-hexahydro-2H-pyrido[2,1-a]isoquinolin-9-yloxy)-acetamide hydrochloride,
(2S,3S,11bS)- and (2R,3R,11bR)-2-(2-amino-10-methoxy-3-m-tolyl-1,3,4,6,7,11b-hexahydro-2H-pyrido[2,1-a]isoquinolin-9-yloxy)-1-morpholin-4-yl-ethanone hydrochloride,
(2R,3S,11bS)- and (2S,3R,11bR)-9-benzyloxy-10-methoxy-3-(4-methyl-pyridin-2-yl)-1,3,4,6,7,11b-hexahydro-2H-pyrido[2,1-a]isoquinolin-2-ylamine,
(2S,3S,11bS) and (2R,3R,11bR)-2-amino-10-methoxy-3-(4-methyl-pyridin-2-yl)-1,3,4,6,7,11b-hexahydro-2H-pyrido[2,1-a]isoquinolin-9-ol,
(2R,3S,11bS)- and (2S,3R,11bR)-2-amino-10-methoxy-3-(4-methyl-pyridin-2-yl)-1,3,4,6,7,11b-hexahydro-2H-pyrido[2,1-a]isoquinolin-9-ol,
(2S,3S,11bS)- and (2R,3R,11bR)-9-(2-benzyloxy-ethoxy)-10-methoxy-3-(4-methyl-pyridin-2-yl)-1,3,4,6,7,11b-hexahydro-2H-pyrido[2,1-a]isoquinolin-2-ylamine,
(2S,3S,11bS)- and (2R,3R,11bR)-2-[2-amino-10-methoxy-3-(4-methyl-pyridin-2-yl)-1,3,4,6,7,11b-hexahydro-2H-pyrido[2,1-a]isoquinolin-9-yloxy]-ethanol,
(2S,3S,11bS)- and (2R,3R,11bR))-9-(2-benzyloxy-1-benzyloxymethyl-ethoxy)-10-methoxy-3-(4-methyl-pyridin-2-yl)-1,3,4,6,7,11b-hexahydro-2H-pyrido[2,1-a]isoquinolin-2-ylamine,
(2S,3S,11bS)- and (2R,3R,11bR)-2-[2-amino-10-methoxy-3-(4-methyl-pyridin-2-yl)-1,3,4,6,7,11b-hexahydro-2H-pyrido[2,1-a]isoquinolin-9-yloxy]-propane-1,3-diol,
(S)-3-[(2S,3S,11bS)- and (2R,3R,11bR)-2-amino-10-methoxy-3-(4-methyl-pyridin-2-yl)-1,3,4,6,7,11b-hexahydro-2H-pyrido[2,1-a]isoquinolin-9-yloxy]-propane-1,2-diol,
(R)-3-[(2S,3S,11bS)- and (2R,3R,11bR) -2-amino-10-methoxy-3-(4-methyl-pyridin-2-yl)-1,3,4,6,7,11b-hexahydro-2H-pyrido[2,1-a]isoquinolin-9-yloxy]-propane-1,2-diol,
(2R,3S,11bS)- and (2S,3R,11bR)-2-[2-amino-10-methoxy-3-(4-methyl-pyridin-2-yl)-1,3,4,6,7,11b-hexahydro-2H-pyrido[2,1-a]isoquinolin-9-yloxy]-1-morpholin-4-yl-ethanone hydrochloride,
(2S,3S,11bS)- and (2R,3R,11bR)-2-[2-amino-10-methoxy-3-(4-methyl-pyridin-2-yl)-1,3,4,6,7,11b-hexahydro-2H-pyrido[2,1-a]isoquinolin-9-yloxy]-1-morpholin-4-yl-ethanone hydrochloride,
(2S,3S,11bS)- and (2R,3R,11bR)-2-[2-amino-10-methoxy-3-(4-methyl-pyridin-2-yl)-1,3,4,6,7,11b-hexahydro-2H-pyrido[2,1a]isoquinolin-9-yloxy]-acetamide hydrochloride,
(2S,3S,11bS)- and (2R,3R,11bR)-2-amino-3-(4-fluoromethyl-pyridin-2-yl)-10-methoxy-1,3,4,6,7,11b-hexahydro-2H-pyrido[2,1-a]isoquinolin-9-yloxy]-acetic acid methyl ester,
(2S,3S,11bS)- and (2R,3R,11bR)-9-benzyloxy-3-(2,5-dimethyl-phenyl)-10-methoxy-1,3,4,6,7,11b-hexahydro-2H-pyrido[2,1-a]isoquinolin-2-ylamine,
   and pharmaceutically acceptable salts thereof.

Furthermore, preferred compounds of formula are those selected from the group consisting of:
rac-(2S,3S,11bS)-8-benzyloxy-9-methoxy-3-m-tolyl-1,3,4,6,7,11b-hexahydro-2H-pyrido[2,1-a]isoquinolin-2-ylamine,
rac-(2S,3S,11bS)-2-amino-9-methoxy-3-m-tolyl-1,3,4,6,7,11b-hexahydro-2H-pyrido[2,1-a]isoquinolin-8-ol,
rac-2-(2-amino-9-methoxy-3-m-tolyl-1,3,4,6,7,11b-hexahydro-2H-pyrido[2,1-a]isoquinolin-8-yloxy)-acetamide, (2S,3S,11bS) and (2R,3S,11bS) diasteromers,
rac-2-((2S,3S,11bS)-2-amino-9-methoxy-3-m-tolyl-1,3,4,6,7,11b-hexahydro-2H-pyrido[2,1-a]isoquinolin-8-yloxy)-1-morpholin-4-yl-ethanone,
rac-2-(2-amino-9-methoxy-3-m-tolyl-1,3,4,6,7,11b-hexahydro-2H-pyrido[2,1-a]isoquinolin-8-yloxy)-1-(4-methyl-piperazin-1-yl)-ethanone, (2S,3S,11bS) and (2R,3S,11bS) diasteromers,
rac-2-((2S,3S,11bS)-2-amino-9-methoxy-3-m-tolyl-1,3,4,6,7,11b-hexahydro-2H-pyrido[2,1-a]isoquinolin-8-yloxy) -N,N-dimethyl-acetamide,
rac-9-methoxy-8-(2-methoxy-ethoxy)-3-m-tolyl-1,3,4,6,7,11b-hexahydro-2H-pyrido[2,1-a]isoquinolin-2-ylamine, (2S,3S,11bS) and (2R,3S,11bS) diasteromers,
rac-2-(2-amino-9-methoxy-3-m-tolyl-1,3,4,6,7,11b-hexahydro-2H-pyrido[2,1-a]isoquinolin-8-yloxy)-ethanol, (2S,3S,11bS) and (2R,3S,11bS) diasteromers,
   and pharmaceutically acceptable salts thereof.

More preferred compounds of the general formula I are those selected from the group consisting of:
(2S,3S,11bS)- and (2R,3R,11bR)-9-(2-amino-ethoxy)-3-(2,5-dimethyl-phenyl)-10-methoxy-1,3,4,6,7,11b-hexahydro-2H-pyrido[2,1-a]isoquinolin-2-ylamine,
(2S,3S,11bS)- and (2R,3R,11bR)-2-amino-3-(2,5-dimethyl-phenyl)-10-methoxy-1,3,4,6,7,11b-hexahydro-2H-pyrido[2,1-a]isoquinolin-9-ol,
(2S,3S,11bS)- and (2R,3R,11bR)-3-(2,5-dimethyl-phenyl)-10-methoxy-9-[2-(1H-tetrazol-5-yl)-ethoxy]-1,3,4,6,7,11b-hexahydro-2H-pyrido[2,1-a]isoquinolin-2-ylamine hydrochloride,
(2S,3S,11bS)- and (2R,3R,11bR)-3-[2-amino-3-(2,5-dimethyl-phenyl)-10-methoxy-1,3,4,6,7,11b-hexahydro-2H-pyrido[2,1-a]isoquinolin-9-yloxy]-propionitrile,
(2S,3S,11bS)- and (2R,3R,11bR)-methanesulfonic acid 2-amino-3-(2,5-dimethyl-phenyl)-10-methoxy-1,3,4,6,7,11b-hexahydro-2H-pyrido[2,1-a]isoquinolin-9-yl ester hydrochloride, (2S,3S, 11bS)- and (2R,3R,11bR)-2-[2-amino-3-(2,5-dimethyl-phenyl)-10-methoxy-1,3,4,6,7,11b-hexahydro-2H-pyrido[2,1-a) isoquinolin-9-yloxy]-ethanol hydrochloride
(2S,3S,11bS)- and (2R,3R,11bR)-[2-amino-3-(2,5-dimethyl-phenyl)-10-methoxy-1,3,4,6,7,11b-hexahydro-2H-pyrido[2,1-a]isoquinolin-9-yloxy]-acetic acid hydrochloride,
(2S,3S,11bS)- and (2R,3R,11bR)-2-[2-amino-3-(2,5-dimethyl-phenyl)-10-methoxy-1,3,4,6,7,11b-hexahydro-2H-pyrido[2,1-a]isoquinolin-9-yloxy]-N-(1H-tetrazol-5-yl)-acetamide hydrochloride,
(2S,3S,11bS)- and (2R,3R,11bR)-2-[2-amino-3-(2,5-dimethyl-phenyl)-10-methoxy-1,3,4,6,7,11b-hexahydro-2H-pyrido[2,1-a]isoquinolin-9-yloxy]-acetamide hydrochloride,
(2S,3S,11bS) - and (2R,3R,11bR)-2-[2-amino-3-(2,5-dimethyl-phenyl)-10-methoxy-1,3,4,6,7,11b-hexahydro-2H-pyrido[2,1-a]isoquinolin-9-yloxy]-N-methyl-acetamide hydrochloride,
(2S,3S,11bS)- and (2R,3R,11bR)-N-[2-Amino-10-methoxy-3-(4-methyl-pyridin-2-yl)-1,3,4,6,7,11b-hexahydro-2H-pyrido[2,1-a]isoquinolin-9-yl]-2-(1H-tetrazol-5-yl)-acetamide hydrochloride,
(2S,3S,11bS)- and (2R,3R,11bR)-10-methoxy-9-methylamino-3-(4-methyl-pyridin-2-yl)-1,3,4,6,7,11b-hexahydro-2H-pyrido[2,1-a]isoquinoline-2,9-diamine hydrochloride,
(2S,3S,11bS)- and (2R,3R,11bR)-[2-amino-10-methoxy-3-(4-methyl-pyridin-2-yl)-1,3,4,6,7,11b-hexahydro-2H-pyrido[2,1-a]isoquinolin-9-yloxy]-acetic acid hydrochloride,
(2S,3S,11bS)- and (2R,3R,11bR)-2-[2-amino-10-methoxy-3-(4-methyl-pyridin-2-yl)-1,3,4,6,7,11b-hexahydro-2H-pyrido[2,1-a]isoquinolin-9-yloxy]-N-(2H-tetrazol-5-yl) -acetamide hydrochloride,
(2S,3S,11bS)- and (2R,3R,11bR)-methanesulfonic acid 2-amino-10-methoxy-3-phenyl-1,3,4,6,7,11b-hexahydro-2H-pyrido[2,1-a]isoquinolin-9-yl ester,
(2S,3S,11bS)- and (2R,3R,11bR)-2-(2-amino-10-methoxy-3-phenyl-1,3,4,6,7,11b-hexahydro-2H-pyrido[2,1-a]isoquinolin-9-yloxy)-N-methyl-acetamide,
(2S,3S,11bS)- and (2R,3R,11bR)-2-(2-amino-10-methoxy-3-phenyl-1,3,4,6,7,11b-hexahydro-2H-pyrido[2,1-a]isoquinolin-9-yloxy)-N,N-dimethyl-acetamide,
(2S,3S,11bS)- and (2R,3R,11bR)-2-amino-10-methoxy-3-m-tolyl-1,3,4,6,7,11b-hexahydro-2H-pyrido[2,1-a]isoquinolin-9-ol,
(2S,3S,11bS)- and (2R,3R,11bR)-2-(2-amino-10-methoxy-3-m-tolyl-1,3,4,6,7,11b-hexahydro-2H-pyrido[2,1-a]isoquinolin-9-yloxy)-ethanol,
(2S,3S,11bS)- and (2R,3R,11bR)-2-(2-amino-10-methoxy-3-m-tolyl-1,3,4,6,7,11b-hexahydro-2H-pyrido[2,1-a]isoquinolin-9-yloxy)-acetamide hydrochloride,

(2S,3S,11bS)- and (2R,3R,11bR)-2-(2-amino-10-methoxy-3-m-tolyl-1,3,4,6,7,11b-hexahydro-2H-pyrido[2,1-a]isoquinolin-9-yloxy)-1-morpholin-4-yl-ethanone hydrochloride,
(2S,3S,11bS) and (2R,3R,11bR)-2-amino-10-methoxy-3-(4-methyl-pyridin-2-yl)-1,3,4,6,7,11b-hexahydro-2H-pyrido[2,1-a]isoquinolin-9-ol,
(2S,3S,11bS)- and (2R,3R,11bR)-2-[2-amino-10-methoxy-3-(4-methyl-pyridin-2-yl)-1,3,4,6,7,11b-hexahydro-2H-pyrido[2,1-a]isoquinolin-9-yloxy]-ethanol,
(2S,3S,11bS)- and (2R,3R,11bR)-2-[2-amino-10-methoxy-3-(4-methyl-pyridin-2-yl)-1,3,4,6,7,11b-hexahydro-2H-pyrido[2,1-a]isoquinolin-9-yloxy]-propane-1,3-diol,
(2S,3S,11bS)- and (2R,3R,11bR)-2-[2-amino-10-methoxy-3-(4-methyl-pyridin-2-yl)-1,3,4,6,7,11b-hexahydro-2H-pyrido[2,1-a]isoquinolin-9-yloxy]-1-morpholin-4-yl-ethanone hydrochloride,
(2S,3S,11bS)- and (2R,3R,11bR)-2-[2-amino-10-methoxy-3-(4-methyl-pyridin-2-yl)-1,3,4,6,7,11b-hexahydro-2H-pyrido[2,1a]isoquinolin-9-yloxy]-acetamide hydrochloride,
(2S,3S,11bS)- and (2R,3R,11bR)-2-amino-3-(4-fluoromethyl-pyridin-2-yl)-10-methoxy-1,3,4,6,7,11b-hexahydro-2H-pyrido[2,1-a]isoquinolin-9-yloxy]-acetic acid methyl ester,
rac-(2S,3S,11bS)-2-amino-9-methoxy-3-m-tolyl-1,3,4,6,7,11b-hexahydro-2H-pyrido[2,1-a]isoquinolin-8-ol,
rac-2-(2-amino-9-methoxy-3-m-tolyl-1,3,4,6,7,11b-hexahydro-2H-pyrido[2,1-a]isoquinolin-8-yloxy)-acetamide, (2S,3S,11bS) and (2R,3S,11bS) diasteromers,
rac-2- ((2S,3S,11bS)-2-amino-9-methoxy-3-m-tolyl-1,3,4,6,7,11b-hexahydro-2H-pyrido[2,1-a]isoquinolin-8-yloxy)-N,N-dimethyl-acetamide,
rac-9-methoxy-8-(2-methoxy-ethoxy)-3-m-tolyl-1,3,4,6,7,11b-hexahydro-2H-pyrido[2,1-a]isoquinolin-2-ylamine, (2S,3S,11bS) and (2R,3S,11bS) diasteromers,
rac-2-(2-amino-9-methoxy-3-m-tolyl-1,3,4,6,7,11b-hexahydro-2H-pyrido[2,1-a]isoquinolin-8-yloxy)-ethanol, (2S,3S,11bS) and (2R,3S,11bS) diasteromers, and pharmaceutically acceptable salts thereof.

Especially preferred compounds of general formula I are those selected from the group consisting of:
(2S,3S,11bS)- and (2R,3R,11bR)-3-(2,5-dimethyl-phenyl)-10-methoxy-9-[2-(1H-tetrazol-5-yl)-ethoxy]-1,3,4,6,7,11b-hexahydro-2H-pyrido[2,1-a]isoquinolin-2-ylamine hydrochloride,
(2S,3S,11bS)- and (2R,3R,11bR)-[2-amino-3- (2,5-dimethyl-phenyl)-10-methoxy-1,3,4,6,7,11b-hexahydro-2H-pyrido[2,1-a]isoquinolin-9-yloxy]-acetic acid hydrochloride,
(2S,3S,11bS)- and (2R,3R,11bR)-2-[2-amino-3-(2,5-dimethyl-phenyl)-10-methoxy-1,3,4,6,7,11b-hexahydro-2H-pyrido[2,1-a]isoquinolin-9-yloxy]-N-(1H-tetrazol-5-yl)-acetamide hydrochloride,
(2S,3S,11bS)- and (2R,3R,11bR)-2-[2-amino-3- (2,5-dimethyl-phenyl)-10-methoxy-1,3,4,6,7,11b-hexahydro-2H-pyrido[2,1-a]isoquinolin-9-yloxy]-acetamide hydrochloride,
(2S,3S,11bS)- and (2R,3R,11bR)-2-[2-amino-3-(2,5-dimethyl-phenyl)-10-methoxy-1,3,4,6,7,11b-hexahydro-2H-pyrido[2,1-a]isoquinolin-9-yloxy]-N-methyl-acetamide hydrochloride,
(2S,3S,11bS)- and (2R,3R,11bR)-N-[2-amino-10-methoxy-3-(4-methyl-pyridin-2-yl)-1,3,4,6,7,11b-hexahydro-2H-pyrido[2,1-a]isoquinolin-9-yl]-2-(1H-tetrazol-5-yl)-acetamide hydrochloride,
(2S,3S,11bS)- and (2R,3R,11bR)-[2-amino-10-methoxy-3-(4-methyl-pyridin-2-yl)-1,3,4,6,7,11b-hexahydro-2H-pyrido[2,1-a]isoquinolin-9-yloxy]-acetic acid hydrochloride,
(2S,3S,11bS)- and (2R,3R,11bR)-2-[2-amino-10-methoxy-3-(4-methyl-pyridin-2-yl)-1,3,4,6,7,11b-hexahydro-2H-pyrido[2,1-a]isoquinolin-9-yloxy]-N-(2H-tetrazol-5-yl)-acetamide hydrochloride,
(2S,3S,11bS)- and (2R,3R,11bR)-methanesulfonic acid 2-amino-10-methoxy-3-phenyl-1,3,4,6,7,11b-hexahydro-2H-pyrido[2,1-a]isoquinolin-9-yl ester,
(2S,3S,11bS)- and (2R,3R,11bR)-2-amino-10-methoxy-3-m-tolyl-1,3,4,6,7,11b-hexahydro-2H-pyrido[2,1-a]isoquinolin-9-ol,
(2S,3S,11bS)- and (2R,3R,11bR)-2-(2-amino-10-methoxy-3-m-tolyl-1,3,4,6,7,11b-hexahydro-2H-pyrido[2,1-a]isoquinolin-9-yloxy)-ethanol,
(2S,3S,11bS)- and (2R,3R,11bR)-2-(2-amino-10-methoxy-3-m-tolyl-1,3,4,6,7,11b-hexahydro-2H-pyrido[2,1-a]isoquinolin-9-yloxy)-acetamide hydrochloride,
(2S,3S,11bS) and (2R,3R,11bR)-2-amino-10-methoxy-3-(4-methyl-pyridin-2-yl)-1,3,4,6,7,11b-hexahydro-2H-pyrido[2,1-a]isoquinolin-9-ol,
(2S,3S,11bS)- and (2R,3R,11bR)-2-[2-amino-10-methoxy-3-(4-methyl-pyridin-2-yl)-1,3,4,6,7,11b-hexahydro-2H-pyrido[2,1-a]isoquinolin-9-yloxy]-ethanol,
rac-2-((2S,3S,11bS)-2-amino-9-methoxy-3-m-tolyl-1,3,4,6,7,11b-hexahydro-2H-pyrido[2,1-a]isoquinolin-8-yloxy)-N,N-dimethyl-acetamide,
and pharmaceutically acceptable salts thereof.

The compounds of formula I have three or more asymmetric carbon atoms and can exist in the form of optically pure enantiomers, mixtures of diastereomers, racemates, or mixtures of diasteroisomeric racemates. The invention embraces all of these forms.

In a preferable embodiment, R¹ and the hydrogen in position 11b of the pyrido[2,1a]isoquinoline backbone are in cis-configuration, whereas the amino group in position 2 of the pyrido[2,1a]isoquinoline backbone is in trans- configuration, i.e.

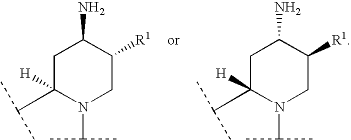

In another preferable embodiment, R¹, the amino group in position 2 and the hydrogen in position 11b of the pyrido[2,1a]isoquinoline backbone are all in cis-configuration, i.e.

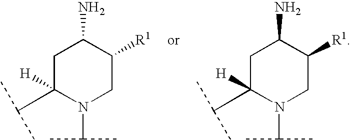

It will be appreciated, that the compounds of general formula (I) in this invention may be derivatized at functional groups to provide derivatives which are capable of conversion back to the parent compound in vivo.

The present invention also relates to a process for the manufacture of compounds of formula I, which process comprises converting a compound of the formula 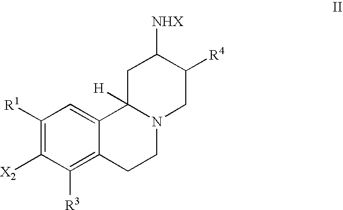
wherein X is hydrogen or tert-butoxycarbonyl, X₂ is OH or —NH₂, R¹ and R⁴ are as defined in herein before and R³ is hydrogen, by side chain transformation into a compound of the formula 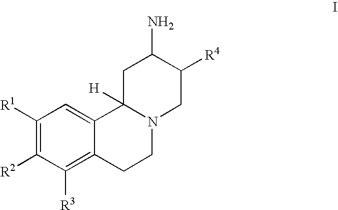
wherein R¹, R² and R⁴ are defined as herein before and R³ is hydrogen, or alternatively, converting a compound of the formula 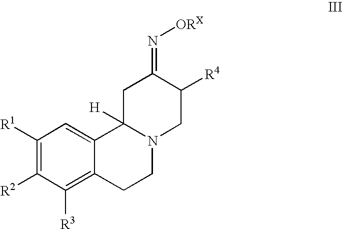
wherein R^{x} is hydrogen or benzyl and R¹ to R⁴ are as defined herein before, by catalytic hydrogen reduction into a compound of the formula 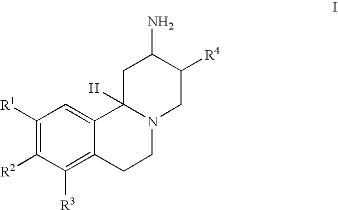
wherein R¹ to R⁴ are defined as herein before, and optionally converting the compound of formula I into a pharmaceutically acceptable salt.

In more detail, the compounds of formula I can be manufactured by the methods given below, by the methods given in the Examples or by analogous methods. Appropriate reaction conditions for the individual reaction steps are known to the person skilled in the art. Starting materials are either commercially available or can be prepared by methods analogous to the methods given below or in the Examples or by methods known in the art.

The compounds of formula I of the present invention can be prepared as indicated in Schemes 1 and 2 below:

The synthesis of pyrido[2,1-a]isoquinoline derivatives of formula 5 is outlined in Scheme I and can be achieved using appropriately substituted 2-phenylethanamines of formula 1 as starting material, compounds well known in the art. The amines of formula 1 can be transformed into the formamides by reaction with formic acid and employing a coupling reagent such as N,N′-carbonyldiimidazole (CDI) or N,N′-dicyclohexyl-carbodiimide (DCC). The formamides are then reacted with POCl₃ or with oxalyl chloride and FeCl₃ to yield the 3,4-dihydroisoquinoline derivatives of formula 2.

In case X₁ is Br, the compound of formula 2 can be transformed into the corresponding benzylamino derivative with the help of a palladium (0) catalyst such as tris(dibenzylideneacetone)dipalladium (0) (Pd₂(dba)₃), rac-2,2′-bis(diphenylphosphino)-1,1′-binaphthyl (BINAP) and sodium tert-butoxide.

Subsequent reaction of 2 with 4-dimethylamino-2-butanone hydrochloride or 4-dimethylamino-3-phenyl-2-butanone hydrochloride yields the ketones of formula 3 wherein R⁴ is hydrogen or phenyl, respectively. Compounds of formula 3 wherein R⁴ is substituted phenyl or pyridyl can be obtained by reacting the compound of formula 3 with R⁴=hydrogen with an appropriate benzene or pyridine under suitable conditions (base, exclusion of oxygen) and the help of a palladium catalyst such as palladium acetate or tris(dibenzylideneacetone)dipalladium (0) (Pd₂(dba)₃)/BINAP.

The ketones of formula 3 are then converted to amino functions by known methods. One possibility is the conversion of the keto group to an oxime of formula 4 using hydroxylamine hydrochloride and sodium acetate or ammonium acetate in a solvent such as ethanol. Oximes can be reduced by e.g. catalytic hydrogenation to the amines of formula 5, wherein X₂ is hydroxy or amino. For example, the hydrogenation can be performed in the presence of a catalyst such as Raney nickel, platinum or palladium in an inert solvent, such as ethanol, at a temperature of about 20 to 80° C.

The 2α, 3β, 11bβ isomer is usually the predominant product which is easily separated from the other stereoisomer by chromatography.

The separation of the enantiomeric mixture in its chiral components can be achieved by chromatography on a chiral phase.

Compounds of formula I wherein R² signifies other residues than hydroxy or amino can be prepared from a compound of formula 5 by subsequent side chain transformation. Such a side chain transformation is for example the formation of an ether. Syntheses of ethers are widely described in literature and well known to the skilled in the art. The transformation can be affected by employing reaction conditions which are commonly utilized in the so-called “Mitsunobu reaction”.

It is typically convenient to couple the compound of formula 5 or the amino protected derivative of formula 6 (whatever is more suitable) with alcohols HO—R^{Y} under conditions employing a phosphine like trialkylphosphine such as tributylphosphine ((n-Bu)₃P), triphenylphosphine (Ph₃P) and the like, and a coupling reagent such as di-tert-butyl-azodicarboxylate, diethyl-azodicarboxylate (DEAD), diisopropyl-azodicarboxylate (DIAD) (optionally polymer bound), tetramethyl azodicarboxamide and the like in a solvent such as tetrahydrofurane (THF), toluene, dichloromethane and the like, to yield compounds of formula 7 or 8 wherein X₃ signifies —O—R^{Y}.

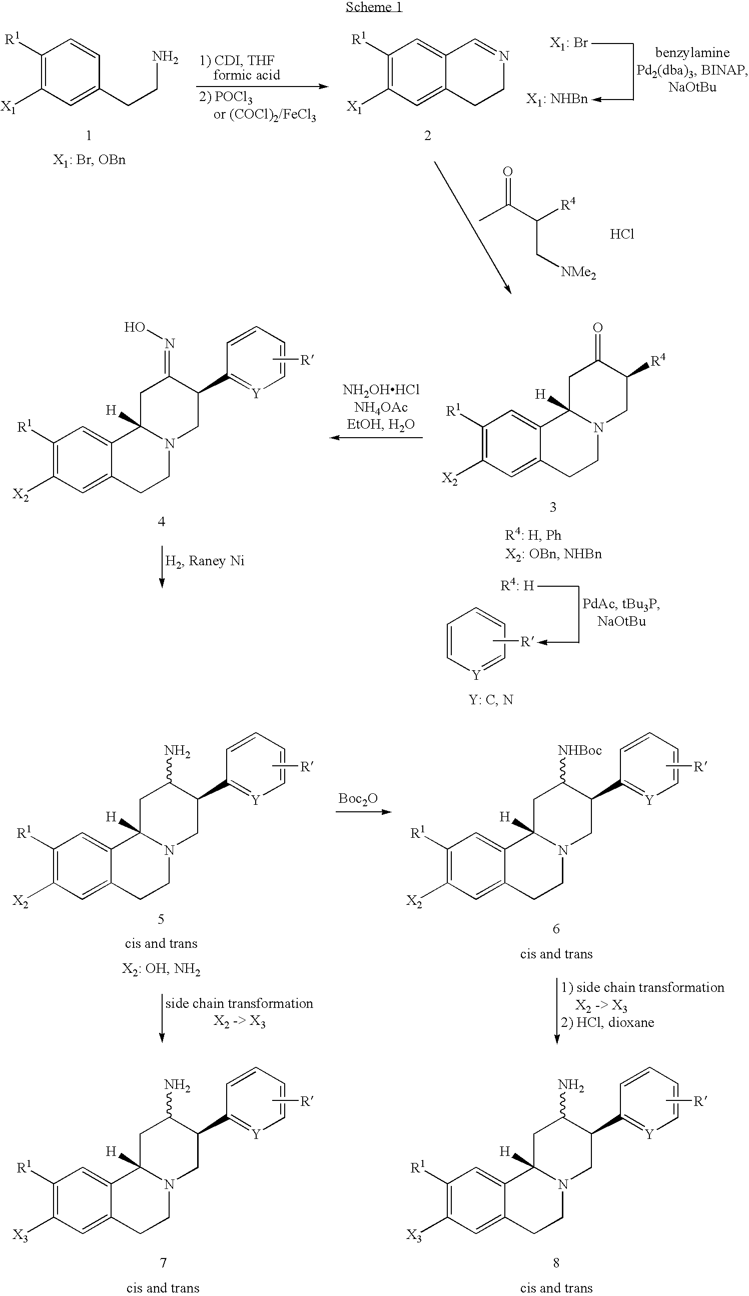

The alcohols HO—R^{Y} (R^{Y} signifies a group selected from lower alkyl, lower hydroxyalkyl, lower cyanoalkyl, lower aminoalkyl, lower alkylaminoalkyl, lower dialkylaminoalkyl, lower alkyl substituted by phenyl which is optionally substituted by one to three groups selected from lower alkyl, lower alkoxy, halogen or lower halogenalkyl, lower alkyl substituted by tetrazolyl, —(CH₂)ₘ—C(O)—NR⁸R⁹, wherein m is 1 or 2 and wherein R⁸ and R⁹ are independently selected from hydrogen, lower alkyl or tetrazolyl, or R⁸ and R⁹ together with the nitrogen atom to which they are attached form a 5- or 6-membered heterocycle which may contain an additional heteroatom selected from N, O or S, and which may be substituted by lower alkyl, —(CH₂)ₙ—COOR¹⁰, wherein n is 1 or 2 and R¹⁰ is hydrogen or lower alkyl, or —(CH₂)ₙ—NH—C(O)—OR¹¹, wherein p is 1 or 2 and wherein R¹¹ is lower alkyl) are either commercially available or accessible by methods described in references or by methods well known in the art.

Amino protected derivatives of compounds of formula 5 such as the tert-butoxycarbonyl (Boc) derivatives 6 can be easily prepared by known methods. Further preferred amino protecting groups are benzyloxycarbonyl (Z) and 9-fluorenylmethoxycarbonyl (Fmoc). Deprotection can be performed by methods known in the art, e.g. the Boc group can be cleaved by employing acidic conditions such as hydrochloric acid in a solvent like dioxane or THF.

A further side chain transformation is the reaction of compounds of formula 6 wherein X₂ is —OH, with an alkylsulfonyl chloride under the presence of a base such as Hunig's base (N,N-diisopropylethylamine, DIPEA) to obtain compounds of formula 8, wherein X₃ is —O—SO₂—R¹², wherein R¹² is lower alkyl.

Another side chain transformation is the amide formation by reacting a compound of formula 6 wherein X₂ is —NH₂ with an appropriate carboxylic acid to obtain a compound of formula 8 wherein X₃ is —NH—CO—(CH₂)_{q}—R¹⁵, wherein q is 1 or 2 and wherein R¹⁵ is lower alkyl or tetrazolyl. This reaction can be carried out under basic conditions, for example by using a base such as triethylamine in an inert solvent like dichloromethane and with the help of a reagent for activating the carboxylic group such as bis(2-oxo-3-oxazolidinyl)phosphinic chloride (BOP-Cl). A further side chain formation is the alkylation of a compound of formula 6 wherein X₂ is —NH₂ to obtain a compound of formula 8 wherein X₃ is —NR¹³R¹⁴, wherein R¹³ is hydrogen or lower alkyl and R¹⁴ is lower alkyl or benzyl.

The synthesis of compounds of formula I according to the present invention wherein R³ is a group other than hydrogen is outlined in Scheme 2.

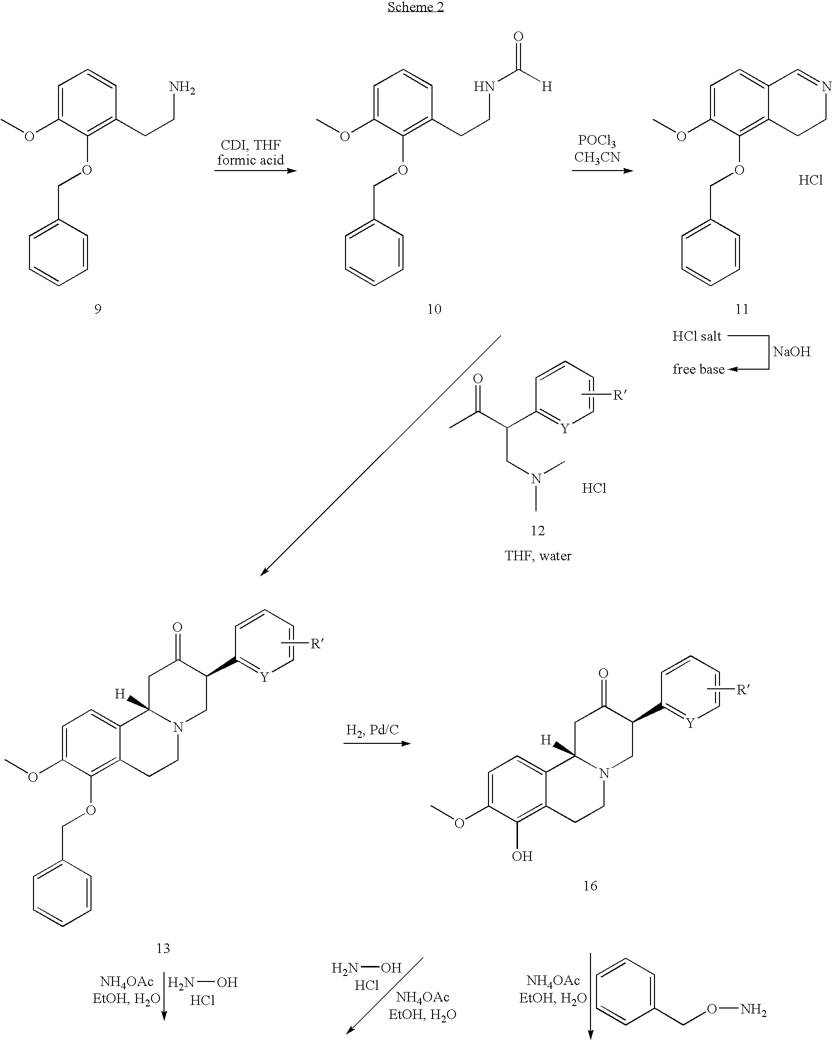

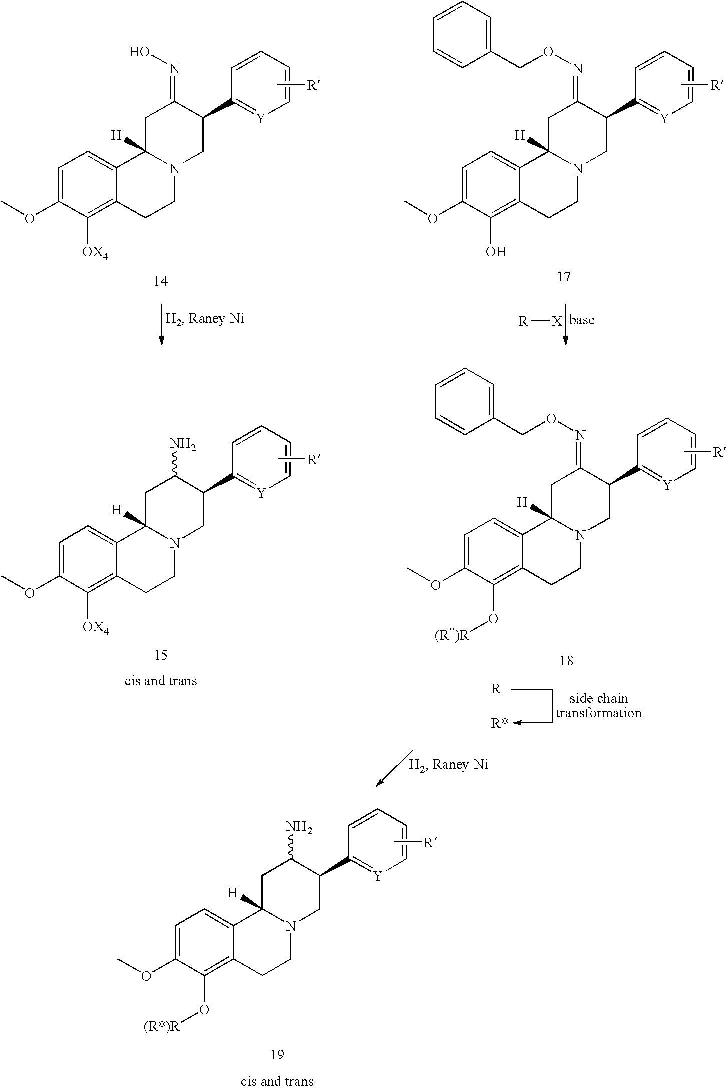

Y means C or N, R′ symbolizes the substituents R⁵, R⁶ and R⁷ as defined herein before, X₄ is hydrogen or benzyl, and R—X is an appropriately substituted alkyihalogenide such as for example bromoacetic acid methyl ester, 2-bromoethyl-benzyl ether or 2-chloroethyl-methyl ether. These alkyl halogenides are commercially available or can be prepared by known methods. The ketones of formula 13 or formula 16 can be transformed in analogy to the method as described in Scheme 1 into the amines of formula 15.

Alternatively, reaction of ketones of formula 16 with O-benzylhydroxylamine and sodium acetate or ammonium acetate leads to O-benzyl-oxime derivatives of formula 17. The compounds of formula 17 are then reacted with an appropriate alkyihalogenide and a strong base such as potassium tert-butylate or sodium tert-butylate in an inert solvent such as dimethylformamide (DMF) to obtain 8-RO-substituted O-benzyl oxime derivatives of formula 18 wherein RO signifies a group R³ as defined herein before or wherein RO can be converted by side chain transformation into a group R*O which corresponds to a group R³ as defined herein before. Finally, the O-benzyl oxime derivatives of formula 18 can be reduced by e.g. catalytic hydrogenation to the amines of formula 19, wherein Y is C or N, R′ symbolizes the substituents R⁵, R⁶ and R⁷ as defined herein before and RO or R*O corresponds to R³ as defined herein before. For example, the hydrogenation can be performed in the presence of a catalyst such as Raney nickel, platinum or palladium in an inert solvent, such as ethanol, at a temperature of about 20 to 80° C.

The separation of the diastereoisomers can be usually done by chromatography. The separation of the enantiomeric mixture in its chiral components can be achieved by chromatography on a chiral phase.

The invention further relates to compounds of formula I as defined above, when manufactured according to a process as defined above.

As described above, the compounds of formula I of the present invention can be used as medicaments for the treatment and/or prophylaxis of diseases which are associated with DPP-IV such as diabetes, particularly non-insulin dependent diabetes mellitus, impaired glucose tolerance, inflammatory bowel disease, Colitis Ulcerosa, Morbus Crohn, obesity, and/or metabolic syndrome or β-cell protection, preferably non-insulin dependent diabetes mellitus and/or impaired glucose tolerance. Furthermore, the compounds of the present invention can be used as diuretic agents or for the treatment and/or prophylaxis of hypertension.

The invention therefore also relates to pharmaceutical compositions comprising a compound as defined above and a pharmaceutically acceptable carrier and/or adjuvant.

Further, the invention relates to compounds as defined above for use as therapeutic active substances, particularly as therapeutic active substances for the treatment and/or prophylaxis of diseases which are associated with DPP-IV such as diabetes, particularly non-insulin dependent diabetes mellitus, impaired glucose tolerance, inflammatory bowel disease, Colitis Ulcerosa, Morbus Crohn, obesity, and/or metabolic syndrome or β-cell protection, preferably for use as therapeutic active substances for the treatment and/or prophylaxis of non-insulin dependent diabetes nellitus and/or impaired glucose tolerance. Furthermore, the invention relates to compounds as defined above for use as diuretic agents or for use as therapeutic active substances for the treatment and/or prophylaxis of hypertension.

In another embodiment, the invention relates to a method for the treatment and/or prophylaxis of diseases which are associated with DPP-IV such as diabetes, particularly non-insulin dependent diabetes mellitus, impaired glucose tolerance, inflammatory bowel disease, Colitis Ulcerosa, Morbus Crohn, obesity, and/or metabolic syndrome or β-cell protection, preferably for the treatment and/or prophylaxis of non-insulin dependent diabetes mellitus and/or impaired glucose tolerance, which method comprises administering a compound as defined above to a human being or animal. Furthermore, the invention relates to a method for the treatment and/or prophylaxis as defined above, wherein the disease is hypertension or wherein a diuretic agent has a beneficial effect.

The invention further relates to the use of compounds as defined above for the treatment and/or prophylaxis of diseases which are associated with DPP-IV such as diabetes, particularly non-insulin dependent diabetes mellitus, impaired glucose tolerance, inflammatory bowel disease, Colitis Ulcerosa, Morbus Crohn, obesity, and/or metabolic syndrome or β-cell protection, preferably for the treatment and/or prophylaxis of non-insulin dependent diabetes mellitus and/or impaired glucose tolerance. Furthermore, the invention relates to the use as defined above, wherein the disease is hypertension or to the use as diuretic agent.

In addition, the invention relates to the use of compounds as defined above for the preparation of medicaments for the treatment and/or prophylaxis of diseases which are associated with DPP-IV such as diabetes, particularly non-insulin dependent diabetes mellitus, impaired glucose tolerance, inflammatory bowel disease, Colitis Ulcerosa, Morbus Crohn, obesity, and/or metabolic syndrome or β-cell protection, preferably for the treatment and/or prophylaxis of non-insulin dependent diabetes mellitus and/or impaired glucose tolerance. Such medicaments comprise a compound as defined above. Furthermore, the invention relates to the use as defined above, wherein the disease is hypertension or the use for the preparation of diuretic agents.

In context with the methods and uses defined above, the following diseases relate to a preferred embodiment: diabetes, particularly non-insulin dependent diabetes mellitus, impaired glucose tolerance, obesity, and/or metabolic syndrome or β-cell protection, preferably non-insulin dependent diabetes mellitus and/or impaired glucose tolerance.

The following tests were carried out in order to determine the activity of the compounds of formula I.

Activity of DPP-IV inhibitors are tested with natural human DPP-IV derived from a human plasma pool or with recombinant human DPP-IV. Human citrate plasma from different donors is pooled, filtered through a 0.2 micron membrane under sterile conditions and aliquots of 1 ml are shock frozen and stored at −120° C. until used. In the colorimetric DPP-IV assay 5 to 10 μl human plasma and in the fluorometric assay 1.0 μl of human plasma in a total assay volume of 100 μl is used as an enzyme source. The cDNA of the human DPP-IV sequence of amino acid 31- to 766, restricted for the N-terminus and the transmembrane domain, is cloned into Pichia pastoris. Human DPP-IV is expressed and purified from the culture medium using conventional column chromatography including size exclusion and anion and cation chromatography. The purity of the final enzyme preparation of Coomassie blue SDS-PAGE is >95%. In the colorimetric DPP-IV assay 20 ng rec.-h DPP-IV and in the fluorometric assay 2 ng rec-h DPP-IV in a total assay volume of 100 μl is used as an enzyme source.

In the fluorogenic assay Ala-Pro-7-amido-4-trifluoromethylcoumarin (Calbiochem No 125510) is used as a substrate. A 20 mM stock solution in 10% DMF/H₂O is stored at −20° C. until use. In IC₅₀ determinations a final substrate concentration of 50 μM is used. In assays to determine kinetic parameters as Kₘ, Vₘₐₓ, Kᵢ, the substrate concentration is varied between 10 μM and 500 μM.

In the colorimetric assay H-Ala-Pro-pNA.HCl (Bachem L-1115) is used as a substrate. A 10 mM stock solution in 10% MeOH/H2O is stored at −20° C. until use. In IC₅₀ determinations a final substrate concentration of 200 μM is used. In assays to determine kinetic parameters as Kₘ, Vₘₐₓ) Kᵢ, the substrate concentration is varied between 100 μM and 2000 μM.

Fluorescence is detected in a Perkin Elmer Luminescence Spectrometer LS 50B at an excitation wavelength of 400 nm and an emission wavelength of 505 nm continuously every 15 seconds for 10 to 30 minutes. Initial rate constants are calculated by best fit linear regression. The absorption of pNA liberated from the calorimetric substrate is detected in a Packard SpectraCount at 405 nm continuously every 2 minutes for 30 to 120 minutes. Initial rate constants are calculated by best fit linear regression.

DPP-IV activity assays are performed in 96 well plates at 37° C. in a total assay volume of 100 μl. The assay buffer consists of 50 mM Tris/HCl-pH 7.8 containing 0.1 mg/ml BSA and 100 mM NaCl. Test compounds are solved in 100% DMSO, diluted to the desired concentration in 10% DMSO/H₂O. The final DMSO concentration in the assay is 1% (v/v). At this concentration enzyme inactivation by DMSO is <5%. Compounds are with (10 minutes at 37° C.) and without pre-incubation with the enzyme. Enzyme reactions are started with substrate application followed by immediate mixing.

IC₅₀ determinations of test compounds are calculated by non-linear best fit regression of the DPP-IV inhibition of at least 5 different compound concentrations. Kinetic parameters of the enzyme reaction are calculated at least 5 different substrate concentrations and at least 5 different test compound concentrations.

The compounds of the present invention exhibit IC₅₀ values of 0.1 nM to 10 μM, more preferably of 0.1-100 nM, as shown in the following table:

| Example | IC₅₀ [μM] |
|---|---|
| 8 | 0.0001 |
| 13 | 0.0011 |
| 48 | 0.0003 |

The compounds of formula I and/or their pharmaceutically acceptable salts can be used as medicaments, e.g. in the form of pharmaceutical preparations for enteral, parenteral or topical administration. They can be administered, for example, perorally, e.g. in the form of tablets, coated tablets, dragees, hard and soft gelatine capsules, solutions, emulsions or suspensions, rectally, e.g. in the form of suppositories, parenterally, e.g. in the form of injection solutions or infusion solutions, or topically, e.g. in the form of ointments, creams or oils. Oral administration is preferred.

The production of the pharmaceutical preparations can be effected in a manner which will be familiar to any person skilled in the art by bringing the described compounds of formula I and/or their pharmaceutically acceptable salts, optionally in combination with other therapeutically valuable substances, into a galenical administration form together with suitable, non-toxic, inert, therapeutically compatible solid or liquid carrier materials and, if desired, usual pharmaceutical adjuvants.

Suitable carrier materials are not only inorganic carrier materials, but also organic carrier materials. Thus, for example, lactose, corn starch or derivatives thereof, talc, stearic acid or its salts can be used as carrier materials for tablets, coated tablets, dragees and hard gelatine capsules. Suitable carrier materials for soft gelatine capsules are, for example, vegetable oils, waxes, fats and semi-solid and liquid polyols (depending on the nature of the active ingredient no carriers might, however, be required in the case of soft gelatine capsules). Suitable carrier materials for the production of solutions and syrups are, for example, water, polyols, sucrose, invert sugar and the like. Suitable carrier materials for injection solutions are, for example, water, alcohols, polyols, glycerol and vegetable oils. Suitable carrier materials for suppositories are, for example, natural or hardened oils, waxes, fats and semi-liquid or liquid polyols. Suitable carrier materials for topical preparations are glycerides, semi-synthetic and synthetic glycerides, hydrogenated oils, liquid waxes, liquid paraffins, liquid fatty alcohols, sterols, polyethylene glycols and cellulose derivatives.

Usual stabilizers, preservatives, wetting and emulsifying agents, consistency-improving agents, flavor-improving agents, salts for varying the osmotic pressure, buffer substances, solubilizers, colorants and masking agents and antioxidants come into consideration as pharmaceutical adjuvants.

The dosage of the compounds of formula I can vary within wide limits depending on the disease to be controlled, the age and the individual condition of the patient and the mode of administration, and will, of course, be fitted to the individual requirements in each particular case. For adult patients a daily dosage of about 1 to 1000 mg, especially about 1 to 100 mg, comes into consideration. Depending on severity of the disease and the precise pharmacokinetic profile the compound could be administered with one or several daily dosage units, e.g. in 1 to 3 dosage units.

The pharmaceutical preparations conveniently contain about 1-500 mg, preferably 1-100 mg, of a compound of formula I.

The following Examples serve to illustrate the present invention in more detail. They are, however, not intended to limit its scope in any manner.

### EXAMPLES

### Example 1

### (2S,3S11bS)- and (2R,3R,11bR)-9-(2-Amino-ethoxy)-3-(2,5-dimethyl-phenyl)-10-methoxy-1,3,4,6,7,11b-hexahydro-2H-pyrido[2,1-a]isoquinolin-2-ylamine

### (3S,11bS)- and (3R,11bR)-9-Benzyloxy-3-(2,5-dimethyl-phenyl)-10-methoxy-1,3,4,6,7,11b-hexahydro-pyrido[2,1-a]isoquinolin-2-one

A mixture of palladium acetate (0.41 g), sodium tert-butoxide (5.4 g) and rac-9-benzyloxy-10-methoxy-1,3,4,6,7,11b-hexahydro-pyrido[2,1-a]isoquinolin-2-one (CAS 68360-33-8; 6.18 g) was dried under high vacuum at 80° C. and flushed with argon three times. Degassed tetrahydrofurane (65 ml) was added at rt under argon. The reaction mixture was stirred for 10 minutes at room temperature, cooled and tri-tert-butyl-phosphine (0.51 g) and 1-bromo-2,5-dimethylbenzene (4.3 g) were added simultaneously with a syringe. The reaction mixture was stirred at 0° C. for 1 h and for another 3 h at ambient temperature under argon. The crude reaction mixture was poured on ice/water, and extracted with CH₂Cl₂. The organic phase was washed with water and brine, dried over magnesium sulfate and concentrated. The residue was purified by column chromatography (silica gel, AcOEt/heptane, 3/2) to yield (3S,11bS)- and (3R,11bR)-9-benzyloxy-3-(2,5-dimethyl-phenyl)-10-methoxy-1,3,4,6,7,11b-hexahydro-pyrido[2,1-a]isoquinolin-2-one (1.9 g) as a white solid.

MS: 442.4 (M+H)⁺

### (3S,11bS)- and (3R,11bR)-9-Benzyloxy-3-(2,5-dimethyl-phenyl)-10-methoxy-1,3,4,6,7,11b-hexahydro-pyrido[2,1-a]isoquinolin-2-one oxime

A suspension of (3S,11bS)- and (3R,11bR)-9-benzyloxy-3-(2,5-dimethyl-phenyl)-10-methoxy-1,3,4,6,7,11b-hexahydro-pyrido[2,1-a]isoquinolin-2-one (218 mg), hydroxylamine hydrochloride (100 mg) and sodium acetate (100 mg) in ethanol (10 ml) was stirred 4 h at room temperature. The mixture was evaporated and the residue crystallized from methanol/water to obtain (3S,11bS)- and (3R,11bR)-9-benzyloxy-3-(2,5-dimethyl-phenyl)-10-methoxy-1,3,4,6,7,11b-hexahydro-pyrido[2,1-a]isoquinolin-2-one oxime (186 mg) as a white solid.

MS: 457.6 (M+H)⁺

### (2S,3S,11bS)- and (2R,3R,11bR)-2-Amino-3-(2,5-dimethyl-phenyl)-10-methoxy-1,3,4,6,7,11b-hexahydro-2H-pyrido[2,1-a]isoquinolin-9-ol

A suspension of (3S,11bS)- and (3R,11bR)-9-benzyloxy-3-(2,5-dimethyl-phenyl)-10-methoxy-1,3,4,6,7,11b-hexahydro-pyrido[2,1-a]isoquinolin-2-one oxime (100 mg), Raney nickel (500 mg) and 1 ml NH₄OH in 5 ml methanol and 5 ml THF was stirred 18 h at room temperature under an H₂ atmosphere. The reaction mixture was filtered, evaporated and chromatographed (silica gel, AcOEt/heptane, 1/1) to provide a white solid (32 mg).

MS: 353.0 (M+H)⁺

### (2S,3S,11bS)-and (2R,3R,11bR)-{2-[2-Amino-3-(2,5-dimethyl-phenyl)-10-methoxy-1,3,4,6,7,11b-hexahydro-2H-pyrido[2,1-a]isoquinolin-9-yloxy]-ethyl} -carbamic acid tert-butyl ester

(2S,3S,11bS)- and (2R,3R,11bR)-2-Amino-3-(2,5-dimethyl-phenyl)-10-methoxy-1,3,4,6,7,11b-hexahydro-2H-pyrido[2,1-a]isoquinolin-9-ol (48 mg), triphenylphosphine (66 mg), di-tert-butyl-azodicarboxylate (62 mg) and Boc-ethanolamine (50 mg) in 4 ml THF were stirred 18 h at room temperature. The reaction mixture was concentrated and chromatographed (silica gel, ethyl acetate/heptane, 1/1) to deliver the product (42 mg).

MS: 496.4 (M+H)⁺

### (2S,3S,11bS)- and (2R,3R,11bR)-9-(2-Amino-ethoxy)-3-(2,5-dimethyl-phenyl)-10-methoxy-1,3,4,6,7,11b-hexahydro-2H-pyrido[2,1-a]isoquinolin-2-ylamine

(2S,3S,11bS)- and (2R,3R,11bR)-{2-[2-amino-3-(2,5-dimethyl-phenyl)-10-methoxy-1,3,4,6,7,11b-hexahydro-2H-pyrido[2,1-a]isoquinolin-9-yloxy]-ethyl}-carbamic acid tert-butyl ester (36 mg) in 1 ml TFA was stirred 1 h at 0° C., then evaporated and chromatographed (silica gel, CH₂Cl₂/MeOH/NH₄OH, 10/1/0.1) to provide the title compound as a white solid (24 mg).

MS: 396.3 (M+H)⁺

### Example 2

### (2S,3S,11bS)- and (2R,3R,11bR)-3-(2,5-Dimethyl-phenyl)-10-methoxy-9-[2-(1H-tetrazol-5-yl)-ethoxyl]-1,3,4,6,7,11b-hexahydro-2H-pyrido[2,1-a]isoquinolin-2-ylamine hydrochloride

### (2S,3S,11bS)- and (2R,3R,11bR)-[3-(2,5-Dimethyl-phenyl)-9-hydroxy-10-methoxy-1,3,4,6,7,11b-hexahydro-2H-pyrido[2,1-a]isoquinolin-2-yl]-carbamic acid tert-butyl ester

A solution of (2S,3S,11bS)- and (2R,3R,11bR)-2-amino-3-(2,5-dimethyl-phenyl)-10-methoxy-1,3,4,6,7,11b-hexahydro-2H-pyrido[2,1-a]isoquinolin-9-ol (1.5 g) and di-tert-butyl-dicarbonate (1.11 g) in 50 ml CH₂Cl₂ was stirred 18 h at room temperature, evaporated and chromatographed (silica gel, ethyl acetate (AcOEt)/heptane, 1/1) to obtain the product as a yellow solid (1.5 g).

MS: 453.3 (M+H)⁺

### (2S,3S,11bS)- and (2R,3R,11bR)-{3-(2,5-Dimethyl-phenyl)-10-methoxy-9-[2-(1H-tetrazol-5-yl)-ethoxy]-1,3,4,6,7,11b-hexahydro-2H-pyrido[2,1-a]isoquinolin-2-yl}-carbamic acid tert-butyl ester

To a solution of (2S,3S,11bS)- and (2R,3R,11bR)-[3-(2,5-dimethyl-phenyl)-9-hydroxy-10-methoxy-1,3,4,6,7,11b-hexahydro-2H-pyrido[2,1-a]isoquinolin-2-yl]-carbamic acid tert-butyl ester (113 mg) in 10 ml THF were added 5-(2-chloro-ethyl)-1H-tetrazole (40 mg) and sodium tert-butylate (29 mg). The reaction mixture was refluxed during 22 h, diluted with AcOEt, washed with water and brine. The aqueous layers were extracted with AcOEt, the organic extracts were dried over magnesium sulfate, evaporated and chromatographed (silica gel, AcOEt/MeOH). The precipitation from methanol/AcOEt delivered the product as a brown solid (45 mg).

MS: 549.5 (M+H)⁺

### (2S,3S,11bS)- and (2R,3R,11bR)-3-(2,5-Dimethyl-phenyl)-10-methoxy-9-[2-(1H-tetrazol-5-yl)-ethoxy]-1,3,4,6,7,11b-hexahydro-2H-pyrido[2,1-a]isoquinolin-2-ylamine; hydrochloride

(2S,3S,11bS)- and (2R,3R,11bR)-{3-(2,5-dimethyl-phenyl)-10-methoxy-9-[2-(1H-tetrazol-5-yl)-ethoxy]-1,3,4,6,7,11b-hexahydro-2H-pyrido[2,1-a]isoquinolin-2-yl}-carbam acid tert-butyl ester (45 mg) in 3 ml dioxane and 1 ml 4M HCl/dioxane was stirred 4 days at room temperature, precipitated with diethyl ether and filtrated to deliver the title compound as a white solid (18 mg).

MS: 449.1 (M+H)⁺

### Example 3

### (2S,3S,11bS)- and (2R,3R,11bR)-3-[2-Amino-3-(2,5-dimethyl-phenyl)-10-methoxy-1,3,4,6,7,11b-hexahydro-2H-pyrido[2,1-a]isoquinolin-9-yloxy]-propionitrile

### (2S,3S,11bS)- and (2R,3R,11bR)-[9-(2-Bromo-ethoxy)-3-(2,5-dimethyl-phenyl)-10-methoxy-1,3,4,6,7,11b-hexahydro-2H-pyrido[2,1-a]isoquinolin-2-yl]-carbamic acid tert-butyl ester

A suspension of (2S,3S,11bS)- and (2R,3R,11bR)-[3-(2,5-dimethyl-phenyl)-9-hydroxy-10-methoxy-1,3,4,6,7,11b-hexahydro-2H-pyrido[2,1-a]isoquinolin-2-yl]-carbamic acid tert-butyl ester (100 mg) in 1,2-dibromo-ethane (1 ml) and NaOH 1M (2 ml) with a few crystals of Bu₄N⁺Br⁻ was vigorously stirred 36 h at 60° C. The reaction mixture was cooled, diluted with AcOEt, washed with water and brine. The aqueous layers were extracted with AcOEt, the organic extracts were dried over magnesium sulfate, evaporated and chromatographed (silica gel, AcOEt/MeOH) to yield 84 mg as a white solid.

MS: 559.5 (M+H)⁺

### (2S,3S,11bS)- and (2R,3R,11bR)-[9-(2-Cyano-ethoxy)-3-(2,5-dimethyl-phenyl)-10-methoxy-1,3,4,6,7,11b-hexahydro-2H-pyrido[2,1-a]isoquinolin-2-yl]-carbamic acid tert-butyl ester

To a solution of (2S,3S,11bS)- and (2R,3R,11bR)-9-(2-bromo-ethoxy)-3-(2,5-dimethyl-phenyl)-10-methoxy-1,3,4,6,7,11b-hexahydro-2H-pyrido[2,1-a]isoquinolin-2-yl]-carbamic acid tert-butyl ester (100 mg) in 5 ml DMF under argon and ice cooling, were added NaCN (23 mg) and tetrakis(triphenylphosphine)palladium (10 mg). The reaction mixture was cooled, poured onto 1M NaOH/ice and extracted with AcOEt. The organic extracts were washed with water and brine dried over magnesium sulfate, evaporated and chromatographied (silica gel, AcOEt/heptane) to yield 54 mg.

MS: 506.5 (M+H)⁺

### (2S,3S,11bS)- and (2R,3R,11bR)-3-[2-Amino-3-(2,5-dimethyl-phenyl)-10-methoxy-1,3,4,6,7,11b-hexahydro-2H-pyrido[2,1-a]isoquinolin-9-yloxy]-propionitrile

(2S,3S,11bS)- and (2R,3R,11bR)-[9-(2-Cyano-ethoxy)-3- (2,5-dimethyl-phenyl)-10-methoxy-1,3,4,6,7,11b-hexahydro-2H-pyrido[2,1-a]isoquinolin-2-yl]-carbamic acid tert-butyl ester (80 mg) were prepared with a similar method as described in example 2c but followed by a basic work-up and a chromatography (silica gel, CH₂Cl₂/MeOH/NH₄OH, 10/1/0.1) to deliver the product as a white solid (14 mg).

MS: 406.5 (M+H)⁺

### Example 4

### (2S,3S, 11bS)- and (2R,3R,11bR)-Methanesulfonic acid 2-amino-3-(2,5-dimethyl-phenyl)-10-methoxy-1,3,4,6,7,11b-hexahydro-2H-pyrido[2,1-a]isoquinolin-9-yl ester; hydrochloride

### (2S,3S,11bS)- and (2R,3R,11bR)-Methanesulfonic acid 2-tert-butoxycarbonylamino-3- (2,5-dimethyl-phenyl)-10-methoxy-1,3,4,6,7,11b-hexahydro-2H-pyrido[2,1-a]isoquinolin-9-yl ester

To a solution of (2S,3S,11bS)- and (2R,3R,11bR)-[3-(2,5-dimethyl-phenyl)-9-hydroxy-10-methoxy-1,3,4,6,7,11b-hexahydro-2H-pyrido[2,1-a]isoquinolin-2-yl]-carbamic acid tert-butyl ester (200 mg) in 5 ml THF under ice cooling were added successively Hunig base (0.51 ml) and methanesulfonyl chloride (0.078 ml). The reaction mixture was stirred 1 h at 0° C., kept 18 h at 4° C., diluted with AcOEt, washed with brine. The aqueous layers were extracted with AcOEt, the combined organic extracts were dried over magnesium sulfate, evaporated and chromatographied (silica gel, AcOEt/MeOH). The precipitation from AcOEt/heptane delivered the product as a white solid (217 mg).

MS: 531.4 (M+H)⁺

### (2S,3S,11bS)- and (2R,3R,11bR)-Methanesulfonic acid 2-amino-3-(2,5-dimethyl-phenyl)-10-methoxy-1,3,4,6,7,11b-hexahydro-2H-pyrido[2,1-a]isoquinolin-9-yl ester; hydrochloride

According to the procedure described in example 2c, (2S,3S,11bS)- and (2R,3R,11bR)-[methanesulfonic acid 2-tert-butoxycarbonylamino-3-(2,5-dimethyl-phenyl)-10-methoxy-1,3,4,6,7,11b-hexahydro-2H-pyrido[2,1-a]isoquinolin-9-yl ester (210 mg) were converted to the title compound, a white solid (70 mg).

MS: 431.4 (M+H)⁺

### Example 5

### (2S,3S,11bS)- and (2R,3R,11bR)-2-[2-Amino-3-(2,5-dimethyl-phenyl)-10-methoxy-1,3,4,6,7,11b-hexahydro-2H-pyrido[2,1-a]isoquinolin-9-yloxy]-ethanol hydrochloride

### (2S,3S,11bS)- and (2R,3R,11bR)-[9-(2-Benzyloxy-ethoxy)-3-(2,5-dimethyl-phenyl)-10-methoxy-1,3,4,6,7,11b-hexahydro-2H-pyrido[2,1-a]isoquinolin-2-yl]-carbamic acid tert-butyl ester

In analogy to example 1d, (2S,3S,11bS)- and (2R,3R,11bR)-[3-(2,5-dimethyl-phenyl)-9-hydroxy-10-methoxy-1,3,4,6,7,11b-hexahydro-2H-pyrido[2,1-a]isoquinolin-2-yl]-carbamic acid tert-butyl ester (144 mg) was converted to the title compound yielding 135 mg of a white solid.

MS: 587.6 (M+H)⁺

### (2S,3S,11bS)- and (2R,3R,11bR)-2-[2-Amino-3-(2,5-dimethyl-phenyl)-10-methoxy-1,3,4,6,7,11b-hexahydro-2H-pyrido[2,1-a]isoquinolin-9-yloxy]-ethanol, hydrochloride

Hydrogenation of (2S,3S,11bS)- and (2R,3R,11bR)-[9-(2-benzyloxy-ethoxy)-3-(2,5-dimethyl-phenyl)-10-methoxy-1,3,4,6,7,11b-hexahydro-2H-pyrido[2,1-a]isoquinolin-2-yl]-carbamic acid tert-butyl ester (110 mg) in 10 ml MeOH and 2 ml of 4M HCl/dioxane with Pd/C 10% followed by a chromatography (silica gel, CH₂Cl₂/MeOH/NH₄OH, 10/1/0.1) provided a solid, which upon treatment with 4M HCl/dioxane yielded the title compound as a white salt (15 mg).

MS: 397.4 (M+H)⁺

### Example 6

### (2S,3S,11bS)- and (2R,3R,11bR)-[2-Amino-3-(2,5-dimethyl-phenyl)-10-methoxy-1,3,4,6,7,11b-hexahydro-2H-pyrido[2,1-a]isoquinolin-9-yloxy]-acetic acid; hydrochloride

### (2S,3S,11bS)- and (2R,3R,11bR)-[2-tert-Butoxycarbonylamino-3-(2,5-dimethyl-phenyl)- 10-methoxy-1,3,4,6,7,11b-hexahydro-2H-pyrido[2,1-a]isoquinolin-9-yloxy]-acetic acid methyl ester

To a solution of (2S,3S,11bS)- and (2R,3R,11bR)-[3-(2,5-dimethyl-phenyl)-9-hydroxy-10-methoxy-1,3,4,6,7,11b-hexahydro-2H-pyrido[2,1-a]isoquinolin-2-yl]-carbamic acid tert-butyl ester (110 mg) in 1 ml THF at 0° C., were added tBuONa (28 mg) and methyl bromoacetate (0.030 ml). The reaction mixture was stirred for 1 h at 0° C. for 2 h at room temperature, diluted with AcOEt and washed with brine. The aqueous layers were extracted with AcOEt, the organic extracts were dried over magnesium sulfate, evaporated and chromatographied (silica gel, AcOEt/heptane) providing (2S,3S,11bS)- and (2R,3R,11bR)-[2-tert-butoxycarbonylamino-3-(2,5-dimethyl-phenyl)-10-methoxy-1,3,4,6,7,11b-hexahydro-2H-pyrido[2,1-a]isoquinolin-9-yloxy]-acetic acid methyl ester as a yellow solid (100 mg).

MS: 525.3 (M+H)⁺

### (2S,3S,11bS)- and (2R,3R,11bR)-[2-tert-Butoxycarbonylamino-3-(2,5-dimethyl-phenyl)-10-methoxy-1,3,4,6,7,11b-hexahydro-2H-pyrido[2,1-a]isoquinolin-9-yloxy]-acetic acid

The saponification of (2S,3S,11bS)- and (2R,3R,11bR)-[2-tert-butoxycarbonylamino-3-(2,5-dimethyl-phenyl)-10-methoxy-1,3,4,6,7,11b-hexahydro-2H pyrido[2,1a]isoquinolin-9-yloxy]-acetic acid methyl ester (380 mg) with lithium hydroxide (125 mg) in 5 ml THF/1 ml water at room temperature and an acidic work up yielded the title compound as an oil (153 mg).

MS: 511.3 (M+H)⁺

### (2S,3S,11bS)- and (2R,3R,11bR)-[2-Amino-3-(2,5-dimethyl-phenyl)-10-methoxy-10-methoxy-1,3,4,6,7,11; b-hexahydro-2H-pyrido[2,1-a]isoquinolin-9-yloxy]-acetic acid hydrochloride

A similar procedure described in example 2c but using (2S,3S,11bS)- and (2R,3R,11bR)-2-tert-butoxycarbonylamino-3-(2,5-dimethyl-phenyl)-10-methoxy-1,3,4,6,7,11b-hexahydro-2H-pyrido[2,1-a]isoquinolin-9-yloxy]-acetic acid (17 mg) and a crystallization (AcOEt/tBuOMe) delivered the title compound as a white solid (18 mg).

MS: 411.3 (M+H)⁺

### Example 7

### (2S,3S,11bS)- and (2R,3R,11bR)-2-[2-Amino-3-(2,5-dimethyl-phenyl)-10-methoxy-1,3,4,6,7,11b-hexahydro-2H-pyrido[2,1-a]isoquinolin-9-yloxy]-N-(1H-tetrazol-5-yl)-acetamide hydrochloride

### (2S,3S,11bS)- and (2R,3R,11bR)-{3-(2,5-Dimethyl-phenyl)-10-methoxy-9-[(1H-tetrazol-5-ylcarbamoyl)-methoxy]-1,3,4,6,7,11b-hexahydro-2H-pyrido[2,1-a]isoquinolin-2-yl}-carbamic acid tert-butyl ester

A solution of (2S,3S,11bS)- and (2R,3R,11bR)-2-tert-butoxycarbonylamino-3-(2,5-dimethyl-phenyl)-10-methoxy-1,3,4,6,7,11b-hexahydro-2H-pyrido[2,1-a]isoquinolin-9-yloxy]-acetic acid (153 mg), Hunig's base (0.17 ml), EDCI (96 mg) and aminotetrazole (85 mg) in 5 ml acetonitrile was stirred for 18 h at ambient temperature. The reaction mixture was diluted with AcOEt, washed with water and brine. The aqueous layers were extracted with AcOEt, the organic extracts were dried over magnesium sulfate, evaporated and chromatographied (silica gel, AcOEt/heptane) to yield (2S,3S,11bS)- and (2R,3R,11bR)-{3-(2,5-dimethyl-phenyl)-10-methoxy-9-[(1H-tetrazol-5-ylcarbamoyl)-methoxy]-1,3,4,6,7,11b-hexahydro-2H-pyrido[2,1-a]isoquinolin-2-yl}-carbamic acid tert-butyl ester as a yellow oil (32 mg).

MS: 578.3 (M+H)⁺

### (2S,3S,11bS) and (2R,3R,11bR)-2-[2-Amino-3-(2,5-dimethyl-phenyl)-10-methoxy-1,3,4,6,7,11b-hexahydro-2H-pyrido[2,1-a]isoquinolin-9-yloxy]-N- (1H-tetrazol-5-yl)-acetamide hydrochloride

This compound was prepared in analogy to example 2c starting from (2S,3S,11bS)- and (2R,3R,11bR)-[3-(2,5-dimethyl-phenyl)-10-methoxy-9-[(1H-tetrazol-5-ylcarbamoyl)-methoxy]-1,3,4,6,7,11b-hexahydro-2H-pyrido[2,1-a]isoquinolin-2-yl}-carbamic acid tert-butyl ester (32 mg) to obtain the tide compound as a white solid (30 mg).

MS: 478.5 (M+H)⁺

### Example 8

### (2S,3S,11bS)- and (2R,3R,11bR)-2-[2-Amino-3-(2,5-dimethyl-phenyl)-10-methoxy-1,3,4,6,7,11b-hexahydro-2H-pyrido[2,1-a]isoquinolin-9-yloxy]-acetamide, hydrochloride

### (2S,3S,11bS)- and (2R,3R,11bR)-[9-Carbamoylmethoxy-3-(2,5-dimethyl-phenyl)-10-methoxy-1,3,4,6,7,11b-hexahydro-2H-pyrido[2,1-a]isoquinolin-2-yl]-carbamic acid tert-butyl ester

(2S,3S,11bS)- and (2R,3R,11bR)-[2-tert-Butoxycarbonylamino-3-(2,5-dimethyl-phenyl)-10-methoxy-1,3,4,6,7,11b-hexahydro-2H-pyrido[2,1-a]isoquinolin-9-yloxy]-acetic acid methyl ester (50 mg) in 1 ml NH₃/MeOH was stirred for 20 h at room temperature, evaporated and chromatographied (silica gel, AcOEt/heptane, 1/1) to yield the product as a yellow oil (35 mg).

MS: 510.8 (M+H)⁺

### (2S,3S,11bS) and (2R,3R,11bR)-2-[2-Amino-3-(2,5-dimethyl-phenyl)-10-methoxy-1,3,4,6,7,11b-hexahydro-2H-pyrido[2,1-a]isoquinolin-9-yloxy]-acetamide hydrochloride

Following the procedure described in example 2c, (2S,3S,11bS)- and (2R,3R,11bR)-2-[2-amino-3-(2,5-dimethyl-phenyl)-10-methoxy-1,3,4,6,7,11b-hexahydro-2H-pyrido[2,1-a]isoquinolin-9-yloxy]-acetamide hydrochloride were obtained after a crystallization (AcOEt/diethylether) as a white solid (28 mg).

MS: 410.6 (M+H)⁺

### Example 9

### (2S,3S,11bS)- and (2R,3R,11bR)-2-[2-Amino-3-(2,5-dimethyl-phenyl)-10-methoxy-1,3,4,6,7,11b-hexahydro-2H-pyrido[2,1-a]isoquinolin-9-yloxy]-N-methyl-acetamide; hydrochloride

In analogy to example 8a and 8b, the title compounds were obtained as a white solid (40 mg).

MS: 424.5 (M+H)⁺

### Example 10

### (2S,3S,11bS)- and (2R,3R,11bR)-N-[2-Amino-10-methoxy-3-(4-methyl-pyridin-2-yl)-1,3,4,6,7,11b-hexahydro-2H-pyrido[2,1-a]isoquinolin-9-yl]-2-(1H-tetrazol-5-yl)-acetamide hydrochloride

### N-[2-(3-Bromo-4-methoxy-phenyl)-ethyl]-formamide

To a solution of 1,1′-carbonyl-diimidazole (7.27 g) in THF (146 ml), formic acid (1.7 ml) in THF (44 ml) was added dropwise. The reaction mixture was stirred for 30 min at room temperature before 10.32 g of 2-(3-bromo-4-methoxy-phenyl)-ethylamine (J. Med. Chem. 1994, 37, 4317-4328) in THF (140 ml) was dropped to the mixture within 40 min. The solution was stirred for 18 h. AcOEt was added and the mixture was washed with 1N HCl and brine. The organic layer was dried over MgSO₄, filtered and concentrated. N-[2-(3-bromo-4-methoxy-phenyl)-ethyl]-formamide was obtained as a white solid (9.42 g).

MS: 257.8 / 259.8 (M+H)⁺

### 6-Bromo-7-methoxy-3,4-dihydro-isoquinoline

To a solution of N-[2-(3-bromo-4-methoxy-phenyl)-ethyl]-formamide (9.00 g) in CH₂Cl₂ (350 ml) was added oxalyl chloride (3.25 ml) dropwise, the reaction mixture was stirred at room temperature for 40 min and then cooled to −20° C. At this temperature, FeCl₃ (6.79 g) was added in one portion. The mixture was allowed to warm slowly to room temperature and was stirred during 18 h. Aqueous 1N HCl (0.7 l) was added to quench the reaction. The mixture was well stirred at room temperature for 1 h, and the layers were separated. The organic layer was washed with brine, dried over MgSO₄, filtered and evaporated. This residue was slurried in MeOH-concentrated H₂SO₄ (19:1, 248 ml) and the mixture was heated at reflux for 2 h. The mixture was cooled and the volatiles were evaporated under vacuum. Water and AcOEt were added. The organic layer was washed twice with 1N HCl. The combined aqueous layers were basified to pH 11. The product was extracted with CH₂Cl₂. The organic layers were washed with brine, dried over MgSO₄, filtered and evaporated. The oil obtained was chromatographed (silica gel, AcOEt) to provide 6-bromo-7-methoxy-3,4-dihydro-isoquinoline (5.29 g).

MS: 239.1/241.0 (M+H)⁺

### Benzyl-(7-methoxy-3,4-dihydro-isoquinolin-6-yl)-amine

To a solution of 6-bromo-7-methoxy-3,4-dihydro-isoquinoline (4.44 g) in 106 ml toluene were added benzylamine (2.4 ml), tris(dibenzylideneacetone)dipalladium (0) (0.071 g), rac-2,2′-bis (diphenylphosphino)-1,1′-binaphtyl (0.132 g) and sodium tert-butoxide (2.49 g). The mixture was heated at 100° C. for 1.4 h under argon and cooled. Tert-butylmethylether and water were added. The aqueous layer was extracted with tert-butylmethylether. The organic layers were washed successively with water, NaHCO₃ and water, dried over MgSO₄, filtered and concentrated yielding an orange oil (4.93 g).

MS: 267.2 (M+H)⁺

### (3R,11bS)- and (3S,11bR)-9-Benzylamino-10-methoxy-3-(4-methyl-pyridin-2-yl)-1,3,4,6,7,11b-hexahydro-pyrido[2,1-a]isoquinolin-2-one

A solution of benzyl-(7-methoxy-3,4-dihydro-isoquinolin-6-yl)-amine (454 mg) and 4-(dimethylamino)-2-butanone hydrochloride (387 mg) in H₂O/THF 3:4 (7 ml) was stirred 1 day at room temperature. Ethyl acetate (AcOEt) was added and the mixture washed with water. The aqueous layer was re-extracted with ethyl acetate. The organic layers were washed successively with water, dried over MgSO₄, filtered and concentrated. Chromatography (silica gel, AcOEt) yielded an orange solid (428 mg).

Under argon, 209 mg of the intermediate obtained above were dissolved in toluene (10 ml). After adding 2-bromo-4-methyl-pyridine (127 mg), tris(dibenzylideneacetone)-dipalladium (0) (2.5 mg), rac-2,2′-bis(diphenylphosphino)-1,1′-binaphtyl (4.4 mg) and sodium tert-butoxide (84 mg) the mixture was heated 4 h at 83° C. The mixture was poured on ice/water and extracted with tert-butylmethylether. The aqueous layer was reextracted with tert-butylmethylether. The combined organic layers were washed successively with water, NaHCO₃ and water, dried over MgSO₄, filtered and concentrated. Chromatography (silica gel, AcOEt/MeOH, 19/1) yielded an orange oil (16 mg).

MS: 428.5 (M+H)⁺

### (3R,11bS)- and (3S,11bR)-(Z or E)-9-Benzylamino-10-methoxy-3-(4-methyl-pyridin-2-yl)-1,3,4,6,7,11b-hexahydro-pyrido[2,1-a]isoquinolin-2-one oxime

To a suspension of (3R,11bS)- and (3S,11bR)-9-benzylamino-10-methoxy-3-(4-methyl-pyridin-2-yl)-1,3,4,6,7,11b-hexahydro-pyrido[2,1-a]isoquinolin-2-one (0.4338 g) in 20 ml EtOH, sodium acetate anhydrous (0.0916 g) and hydroxylamine hydrochloride (0.0776 g) were added. The mixture was stirred 17 h at room temperature. Then 13 ml of water and 13 ml of a saturated solution of NaHCO₃ were added. The solvent was partially evaporated. The precipitated solid was filtered off and washed with water and heptane. (3R,11bS)- and (3S,11bR)-(Z or E)-9-benzylamino-10-methoxy-3-(4-methyl-pyridin-2-yl)-1,3,4,6,7,11b-hexahydro-pyrido[2,1-a]isoquinolin-2-one oxime were obtained as a yellow solid (0.45 g).

MS: 443.5 (M+H)⁺

### (2S,3S,11bS)- and (2R,3R,11bR)-N9-Benzyl-10-methoxy-3-(4-methyl-pyridin-2yl)-1,3,4,6,7,11b-hexahydro-2H-pyrido[2,1-a]isoquinolin-2,9-diamine hydrochloride and (2S,3R,11RS)- and (2R,3S,11bS)-N9-benzyl-10-methoxy-3-(4-methyl-pyridin-2-yl)-1,3,4,6,7,11b-hexahydro-2H-pyrido[2,1-a]isoquinolin-2,9-diamine

To a suspension of Raney nickel (0.65 g) in 6.5 ml EtOH and 6.5 ml dioxane, (3R,11bS)- and (3S,11bR)-(9-benzylamino-10-methoxy-3-(4-methyl-pyridin-2-yl)-1,3,4,6,7,11b-hexahydro-pyrido[2,1-a]-isoquinolin-2-one oxime (0.17 g) and a concentrated ammonium hydroxide solution (0.65 ml) were added. The mixture was stirred under H₂ at room temperature for 22 h. The catalyst was filtered over dicalite and the filtrate evaporated. The yellow solid obtained was chromatographed (silica gel, CH₂Cl₂/MeOH/NH₄OH, 9/1/0.05) yielding two racemates, (2S,3S,11bS)- and (2R,3R,11bR)-N9-benzyl-10-methoxy-3-(4-methyl-pyridin-2-yl)-1,3,4,6,7,11b-hexahydro-2H-pyrido[2,1-a]isoquinolin-2,9-diamine (30 mg) and (2S,3R,11RS)- and (2R,3S,11bS)-N9-benzyl-10-methoxy-3-(4-methyl-pyridin-2-yl)-1,3,4,6,7,11b-hexahydro-2H-pyrido[2,1-a]isoquinolin-2,9-diamine (152 mg), respectively. The 2,3-trans-isomer was further treated with 4M HCl/dioxane to provide the salt as a yellow solid (0.15 g).

MS: 429.6 (M+H)⁺

### (2S,3S,11bS)- and (2R,3R,11bR)-([9-Benzylamino-10-methoxy-3-(4-methyl-pyridin-2-yl)-1,3,4,6,7,11b-hexahydro-2H-pyrido[2,1-a]isoquinolin-2-yl]-carbamic acid tert-butyl ester

To a solution of (2S,3S,11bS)- and (2R,3R,11bR)-N9-benzyl-10-methoxy-3-(4-methyl-pyridin-2-yl)-1,3,4,6,7,11b-hexahydro-2H-pyrido[2,1-a]isoquinolin-2,9-diamine (250 mg) in 2.5 ml CH₂Cl₂, Boc₂O (128 mg) was added. The reaction mixture was stirred for 2 h at room temperature, evaporated and chromatographied (SPE Isolute Flash NH₂, AcOEt/heptane, 2/1) to yield the product as a white solid (210 mg).

MS: 529.3 (M+H)⁺

### (2S,3S,11bS)- and (2R,3R,11bR)-[9-Amino-10-methoxy-3-(4-methyl-pyridin-2-yl)1,3,4,6,7,11b-hexahydro-2H-pyrido[2,1-a]isoquinolin-2-yl]-carbamic acid tert-butyl ester

A suspension of (2S,3S,11bS)- and (2R,3R,11bR)-[9-benzylamino-10-methoxy-3-(4-methyl-pyridin-2-yl)-1,3,4,6,7,11b-hexahydro-2H-pyrido[2,1-a]isoquinolin-2-yl]-carbamic acid tert-butyl ester (0.1962 g) and palladium on activated charcoal (10% Pd, 0.325 g) in 6 ml MeOH and 4 ml CH₂Cl₂ was stirred under hydrogen for 22 h at room temperature. The mixture was filtered over dicalite, under argon, the filtrate was evaporated and chromatographed (silica gel, AcOEt/MeOH, 4/1) providing (2S,3S,11bS)- and (2R,3R,11bR)-[9-amino-10-methoxy-3-(4-methyl-pyridin-2-yl)-1,3,4,6,7,11b-hexahydro-2H-pyrido[2,1-a]isoquinolin-2-yl]-carbamic acid tert-butyl ester (66 mg).

MS: 439.4 (M+H)⁺

### (2S,3S,11bS)- and (2R,3R,11bR)-N-[2-Amino-10-methoxy-3-(4-methyl-pyridin-2-yl)-1,3,4,6,7,11b-hexahydro-2H-pyrido[2,1-a]isoquinolin-9-yl]-2-(1H-tetrazol-5-yl)-acetamide hydrochloride

To a solution of (2S,3S,11bS)- and (2R,3R,11bR)-[9-amino-10-methoxy-3-(4-methyl-pyridin-2-yl) 1,3,4,6,7,11b-hexahydro-2H-pyrido[2,1-a]isoquinolin-2-yl]-carbamic acid tert-butyl ester (17 mg), 1H-tetrazole-5-acetic acid (6 mg) and triethylamine (0.013 ml) in dichloromethane (1 ml) at 0° C., bis(2-oxo-3-oxazolidinyl)phosphinic chloride (12 mg) was added. After 36 h at room temperature the solvent was evaporated. HPLC (RP-8 (Lichroprep, 40-63 μm, Merck), H₂O/MeCN) provided upon evaporation a white solid.

This residue (31 mg) was dissolved in 3 ml dioxane and treated with 4M HCl/dioxane during 18 h at room temperature. The reaction mixture was evaporated and purified by HPLC (Combi HT SB C18, 50 mm, 5 mm, H₂O/NEt₃/MeCN). Lyophilisation and treatment of the residue with 4M HCl/dioxane provided (2S,3S,11bS)- and (2R,3R,11bR)-N-[2-amino-10-methoxy-3-(4-methyl-pyridin-2-yl)-1,3,4,6,7,11b-hexahydro-2H-pyrido[2,1-a]isoquinolin-9-yl]-2-(2H-tetrazol-5-yl)-acetamide hydrochloride as a white solid (4 mg).

MS: 449.2 (M+H)⁺

### Example 11

### (2S,3S,11bS)- and (2R,3R,11bR)-10-Methoxy-9-methylamino-3-(4-methyl-pyridin-2-yl)-1,3,4,6,7,11b-hexahydro-2H-pyrido[2,1-a]isoquinoline-2,9-diamine hydrochloride

(2S,3S,11bS)- and (2R,3R,11bR)-[10-Methoxy-9-methylamino-3-(4-methyl-pyridin-2-yl)-1,3,4,6,7,11b-hexahydro-2H-pyrido[2,1-a]isoquinolin-2-yl]-carbamic acid tert-butyl ester (17 mg) obtained as a by-product in example 10h were treated according to the procedure described in example 2c providing the title compound as a white solid (9 mg).

MS: 353.4 (M+H)⁺

### Example 12

### (2S,3S,11bS)- and (2R,3R11bR)-[2-Amino-10-methoxy-3-(4-methyl-pyridin-2-yl)-1,3,4,6,7,11b-hexahydro-2H-pyrido[2,1-a]isoquinolin-9-yloxy]-acetic acid, hydrochloride

### (3R,11bS)- and (3S,11bR)-9-Benzyloxy-10-methoxy-3-(4-methyl-pyridin-2-yl)-1,3,4,6,7,11b-hexahydro-pyrido[2,1-a]isoquinolin-2-one

The compound was synthesized from rac-9-benzyloxy-10-methoxy-1,3,4,6,7,11b-hexahydro-pyrido[2,1-a]isoquinolin-2-one (CAS 68360-33-8; 30 g) and 2-bromo-4-methylpyridne according to the procedure described in example la to yield a yellow solid (9.9 g).

MS: 429.6 (M+H)⁺

### (3R,11bS)- and (3S,11bR)-9-Benzyloxy-10-methoxy-3-(4-methyl-pyridin-2-yl)-1,3,4,6,7,11b-hexahydro-pyrido[2,1-a]isoquinolin-2-one oxime

(3R,11bS)- and (3S,11bR)-9-benzyloxy-10-methoxy-3-(4-methyl-pyridin-2-yl)-1,3,4,6,7,11b-hexahydro-pyrido[2,1-a]isoquinolin-2-one (8.2 g) was treated under the same conditions described in example 1b providing the title compound as a white solid (8.59 g).

MS: 444.0 (M+H)⁺

### (2S,3S,11bS)- and (2R,3R,11bR)-9-Benzyloxy-10-methoxy-3-(4-methyl-pyridin-2-yl)-1,3,4,6,7,11b-hexahydro-2H-pyrido[2,1-a]isoquinolin-2-ylamine

(3R,11bS)- and (3S,11bR)-9-Benzyloxy-10-methoxy-3-(4-methyl-pyridin-2-yl)-1,3,4,6,7,11b-hexahydro-pyrido[2,1-a]isoquinolin-2-one oxime (2.55 g) were treated under the conditions described as in example 1c to deliver the title compound as a foam (1.08 g).

MS: 430.4 (M+H)⁺

### (2S,3S,11bS)- and (2R,3R,11bR)-[9-Benzyloxy-10-methoxy-3-(4-methyl-pyridin-2-yl)-1,3,4,6,7,11b-hexahydro-2H-pyrido[2,1-a]isoquinolin-2-yl]-carbamic acid tert-butyl ester

(2S,3S,11bS)- and (2R,3R,11bR)-9-Benzyloxy-10-methoxy-3-(4-methyl-pyridin-2-yl)-1,3,4,6,7,11b-hexahydro-2H-pyrido[2,1-a]isoquinolin-2-ylamine (1.08 g) was converted to the title compound, a white solid (520 mg), using the procedure described for example 2a.

MS: 530.4 (M+H)⁺

### (2S,3S,11bS)- and (2R,3R,11bR)-[9-Hydroxy-10-methoxy-3-(4-methyl-pyridin-2-yl)-1,3,4,6,7,11b-hexahydro-2H-pyrido[2,1-a]isoquinolin-2-yl]-carbamic acid tert-butyl ester

A suspension of (2S,3S,11bS)- and (2R,3R,11bR)-[9-benzyloxy-10-methoxy-3-(4-methyl-pyridin-2-yl)-1,3,4,6,7,11b-hexahydro-2H-pyrido[2,1-a]isoquinolin-2-yl]-carbamic acid tert-butyl ester (440 mg) and palladium on activated charcoal (10% Pd, 0.060 g) in 15 ml MeOH and 15 ml AcOEt, was stirred under hydrogen for 3 h at room temperature. The mixture was filtered over dicalite under argon and the filtrate was evaporated providing (2S,3S,11bS)- and (2R,3R,11bR)-[9-hydroxy-10-methoxy-3-(4-methyl-pyridin-2-yl)-1,3,4,6,7,11b-hexahydro-2H-pyrido[2,1-a]isoquinolin-2-yl]-carbamic acid tert-butyl ester as a white solid (327 mg).

MS: 440.4 (M+H)⁺

### (2S,3S,11bS)- and (2R,3R,11bR)-[2-Amino-10-methoxy-3-(4-methyl-pyridin-2-yl)-1,3,4,6,7,11b-hexahydro-2H-pyrido[2,1-a]isoquinolin-9-yloxy]-acetic acid hydrochloride

(2S,3S,11bS)- and (2R,3R,11bR)-[2-Amino-10-methoxy-3-(4-methyl-pyridin-2-yl)-1,3,4,6,7,11b-hexahydro-2H-pyrido[2,1-a]isoquinolin-9-yloxy]-acetic acid hydrochloride were prepared with the procedure described in example 6a to 6c, but starting from (2S,3S,11bS)- and (2R,3R,11bR)-[9-hydroxy-10-methoxy-3-(4-methyl-pyridin-2-yl)-1,3,4,6,7,11b-hexahydro-2H-pyrido[2,1-a]isoquinolin-2-yl]-carbamic acid tert-butyl ester. The product obtained was a white solid (38 mg).

MS (ISN): 396.2 (M−H)⁻

### Example 13

### (2S,3S11bS)- and (2R,3R,11bR)-2-[2-Amino-10-methoxy-3-(4-methyl-pyridin-2-yl)-1,3,4,6,7,11b-hexahydro-2H-pyrido[2,1-a]isoquinolin-9-yloxy]-N-(2H-tetrazol-5-yl)-acetamide hydrochloride

### (2S,3S,11bS)- and (2R,3R,11bR)-10-Methoxy-3-(4-methyl-pyridin-2-yl)-9-[(2H-tetrazol-5-ylcarbamoyl)-methoxy]-1,3,4,6,7,11b-hexahydro-2H-pyrido[2,1-a]isoquinolin-2-yl}-carbamic acid tert-butyl ester

The procedure described in example 7a, but using (2S,3S,11bS)- and (2R,3R,11bR)-[2-tert-butoxycarbonylamino-10-methoxy-3-(4-methyl-pyridin-2-yl)-1,3,4,6,7,11b-hexahydro-2H-pyrido[2,1-a]isoquinolin-9-yloxy]-acetic acid (160 mg), delivered (2S,3S,11bS)- and (2R,3R,11 bR)-10-methoxy-3-(4-methyl-pyridin-2-yl)-9-[(2H-tetrazol-5-ylcarbamoyl)-methoxy]-1,3,4,6,7,11b-hexahydro-2H-pyrido[2,1-a]isoquinolin-2-yl}-carbamic acid tert-butyl ester as a yellow solid (37 mg).

MS: 565.5 (M+H)⁺

### (2S,3S,11bS)- and (2R,3R,11bR)-2-[2-Amino-10-methoxy-3-(4-methyl-pyridin-2-yl)-1,3,4,6,7,11b-hexahydro-2H-pyrido[2,1-a]isoquinolin-9-yloxy]-N-(2H-tetrazol-5-yl)-acetamide hydrochloride

The title compound was prepared according to the procedure described in example 7b to yield a yellow solid (29 mg).

MS: 465.4 (M+H)⁺

### Example 14

### (2S,3S,11bS)- and (2R,3R,11bR)-Methanesulfonic acid 2-amino-10-methoxy-3-phenyl-1,3,4,6,7,11b-hexahydro-2H-pyrido[2,1-a]isoquinolin-9-yl ester

### (3S,11bS)- and (3R,11bR)-9-Benzyloxy-10-methoxy-3-phenyl-1,3,4,6,7,11b-hexahydro-pyrido[2,1-a]isoquinolin-2-one

A suspension of 6-benzyloxy-7-methoxy-3,4-dihydro-isoquinoline (CAS 68360-22-5, 4 g) and benzeneethanaminium-β-acetyl-N,N,N-trimethyl-iodide (CAS 31034-99-8; 7.48 g) in 100 ml ethanol and 0.75 ml NaOH 1M was heated at reflux during 2 h. Under cooling, the product precipitated as a yellow solid (3.07 g).

MS: 414.2 (M+H)⁺

### (3S, 11bS)- and (3R,11bR)-9-Benzyloxy-10-methoxy-3-phenyl-1,3,4,6,7,11b-hexahydro-pyrido[2,1-a]isoquinolin-2-one oxime

(3S,11bS)- and (3R,11bR)-9-Benzyloxy-10-methoxy-3-phenyl-1,3,4,6,7,11b-hexahydro-pyrido[2,1-a]isoquinolin-2-one (358 mg) were treated as described in example 1b to provide the title compound as a white solid (358 mg).

MS: 429.6 (M+H)⁺

### (2S,3S,11bS)- and (2R,3R,11bR)-9-Benzyloxy-10-methoxy-3-phenyl-1,3,4,6,7,11b-hexahydro-2H-pyrido[2,1-a]isoquinolin-2-ylamine and

### (2R,3S,11bS)- and (2S,3R,11bR)-9-benzyloxy-10-methoxy-3-phenyl-1,3,4,6,7,11b-hexahydro-2H-pyrido[2,1-a]isoquinolin-2-ylamine

(3S,11bS)- and (3R,11bR)-9-Benzyloxy-10-methoxy-3-phenyl-1,3,4,6,7,11b-hexahydro-pyrido[2,1-a]isoquinolin-2-one oxime (350 mg) were hydrogenated under the same conditions as described in example 1c. Two diastereoisomers were separated by chromatography (silica gel, CH₂Cl₂/MeOH/NH₄OH, 9/1/0.05), (2S,3S,11bS)- and (2R,3R,11bR)-9-benzyloxy-10-methoxy-3-phenyl-1,3,4,6,7,11b-hexahydro-2H-pyrido[2,1-a]isoquinolin-2-ylamine (171 mg, 50%, R_{f}=0.25) and (2R,3S,11bS)- and (2S,3R,11bR)-9-benzyloxy-10-methoxy-3-phenyl-1,3,4,6,7,11b-hexahydro-2H-pyrido[2,1-a]isoquinolin-2-ylamine (27 mg, 8%, R_{f}=0.5).

MS: 415.5 (M+H)⁺

### (2S,3S,11bS)- and (2R,3R,11bR)-(9-Benzyloxy-10-methoxy-3-phenyl-1,3,4,6,7,11b-hexahydro-2H-pyrido[2,1-a]isoquinolin-2-yl)-carbamic acid tert-butyl ester

(2S,3S,11bS)- and (2R,3R,11bR)-9-Benzyloxy-10-methoxy-3-phenyl-1,3,4,6,7,11b-hexahydro-2H-pyrido[2,1-a]isoquinolin-2-ylamine (200 mg) were converted to the title compound using the procedure described in example 2a. (2S,3S,11bS)- and (2R,3R,11bR)-(9-Benzyloxy-10-methoxy-3-phenyl-1,3,4,6,7,11b-hexahydro-2H-pyrido[2,1-a]isoquinolin-2-yl)-carbamic acid tert-butyl ester were obtained as a white solid (243 mg).

MS: 515.5 (M+H)⁺

### (2S,3S,11bS)- and (2R,3R,11bR)-(9-Hydroxy-10-methoxy-3-phenyl-1,3,4,6,7,11b-hexahydro-2H-pyrido[2,1-a]isoquinolin-2-yl)-carbamic acid tert-butyl ester

Starting from (2S,3S,11bS)- and (2R,3R,11bR)-(9-benzyloxy-10-methoxy-3-phenyl-1,3,4,6,7,11b-hexahydro-2H-pyrido[2,1-a]isoquinolin-2-yl)-carbamic acid tert-butyl ester (2.6 g) and using the same procedure as for compound 2c, the title compound was obtained as a yellow solid (1.58 g).

MS: 425.5 (M+H)⁺

### (2S,3S,11bS)- and (2R,3R,11bR)-Methanesulfonic acid 2-tert-butoxycarbonylamino-10-methoxy-3-phenyl-1,3,4,6,7,11b-hexahydro-2H-pyrido[2,1-a]isoquinolin-9-yl ester

To a solution of (2S,3S,11bS)- and (2R,3R,11bR)-(9-hydroxy-10-methoxy-3-phenyl-1,3,4,6,7,11b-hexahydro-2H-pyrido[2,1-a]isoquinolin-2-yl)-carbamic acid tert-butyl ester (130 mg) in 5 ml THF under ice cooling were successively added potassium tert-butylate (51 mg) and methanesulfonyl chloride (0.031 ml). The reaction mixture was stirred at reflux 18 h, diluted with CH₂Cl₂, washed with NaHCO₃ and brine. The aqueous layers were extracted with CH₂Cl₂, the organic extracts were dried over magnesium sulfate, evaporated and precipitated from tBuOMe whereby the product was delivered as a yellow solid (148 mg).

MS: 503.4 (M+H)⁺

### (2S,3S,11bS)- and (2R,3R,11bR)-Methanesulfonic acid 2-amino-10-methoxy-3-phenyl-1,3,4,6,7,11b-hexahydro-2H-pyrido[2,1-a]isoquinolin-9-yl ester

(2S,3S,11bS)- and (2R,3R,11bR)-Methanesulfonic acid 2-tert-butoxycarbonyl-amino-10-methoxy-3-phenyl-1,3,4,6,7,11b-hexahydro-2H-pyrido[2,1-a]isoquinolin-9-yl ester (140 mg) in 2 ml 4M HCl/dioxane was stirred 2 days at room temperature. The mixture was evaporated, diluted with CH₂Cl₂ and 1M NaOH. The aqueous layers were extracted with CH₂Cl₂, the organic extracts were dried over magnesium sulfate, evaporated and precipitation from tBuOMe delivered the product as a yellow solid (76 mg).

MS: 403.5 (M+H)⁺

### Example 15

### (2S,3S,11bS)- and (2R,3R,11bR)-2-(2-Amino-10-methoxy-3-phenyl-1,3,4,6,7,11b-hexahydro-2H-pyrido[2,1-a]isoquinolin-9-yloxy)-ethanol

### (2S,3S,11bS)- and (2R,3R,11bR)-[9-(2-Benzyloxy-ethoxy)-10-methoxy-3-phenyl-1,3,4,6,7,11b-hexahydro-2H-pyrido[2,1-a]isoquinolin-2-yl]-carbamic acid tert-butyl ester

A suspension of (2S,3S,11bS)- and (2R,3R,11bR)-(9-hydroxy-10-methoxy-3-phenyl-1,3,4,6,7,11b-hexahydro-2H-pyrido[2,1-a]isoquinolin-2-yl)-carbamic acid tert-butyl ester (100 mg), benzyl 2-bromoethyl ether (0.044 ml) and potassium tert-butylate (39 mg) in 4 ml THF was refluxed during 20 h. The mixture was diluted with CH₂Cl₂ and NaHCO₃. The aqueous layers were extracted with CH₂Cl₂, the organic extracts were dried over magnesium sulfate, evaporated and precipitation from tBuOMe delivered the product as a yellow solid (57 mg).

MS: 559.7 (M+H)⁺

### (2S,3S,11bS)- and (2R,3R,11bR)-9-(2-Benzyloxy-ethoxy)-10-methoxy-3-phenyl-1,3,4,6,7,11b-hexahydro-2H-pyrido[2,1-a]isoquinolin-2-ylamine

(2S,3S,11bS)- and (2R,3R,11bR)-[9-(2-Benzyloxy-ethoxy)-10-methoxy-3-phenyl-1,3,4,6,7,11b-hexahydro-2H-pyrido[2,1-a]isoquinolin-2-yl]-carbamic acid tert-butyl ester (50 mg) was treated according to the procedure described in example 14g providing the product as a yellow solid (19 mg).

MS: 459.6 (M+H)⁺

### (2S,3S,11bS)- and (2R,3R,11bR)-2-(2-Amino-10-methoxy-3-phenyl-1,3,4,6,7,11b-hexahydro-2H-pyrido[2,1-a]isoquinolin-9-yloxy)-ethanol

A solution of (2S,3S,11bS)- and (2R,3R,11bR)-[9-(2-benzyloxy-ethoxy)-10-hexahydro-3-phenyl-1,3,4,6,7,11b-hexahydro-2H-pyrido[2,1-a]isoquinolin-2-yl]-carbamic acid tert-butyl ester (280 mg) in 7 ml methanol was treated with HCl/dioxane and Pd/C 10% under an H₂ atmosphere during 3 h. The reaction mixture was filtered (dicalite), evaporated, and chromatographed (silica gel, AcOEt/MeOH/NH₄OH, 95/4/1) to yield a yellow solid (107 mg).

MS: 369.4 (M+H)⁺

### Example 16

### (2S,3S,11bS)- and (2R,3R,11bR)-9-(2-Amino-ethoxy)-10-methoxy-3-phenyl-1,3,4.6,7,11b-hexahydro-2H-pyrido[2,1-a]isoquinolin-2-ylamine

### (2S,3S,11bS)- and (2R,3R,11bR)-[9-(2-tert-Butoxycarbonylamino-ethoxy)-10-methoxy-3-phenyl-1,3,4,6,7,11b-hexahydro-2H-pyrido[2,1-a]isoquinolin-2-yl]-carbamic acid tert-butyl ester

This compound was prepared according to the method described in example 15a starting from (2S,3S,11bS)- and (2R,3R,11bR)-(9-hydroxy-10-methoxy-3-phenyl-1,3,4,6,7,11b-hexahydro-2H-pyrido[2,1-a]isoquinolin-2-yl)-carbamic acid tert-butyl ester (300 mg) and 2-boc-amino-ethylbromide to yield a white solid (203 mg).

MS: 568.6 (M+H)⁺

### (2S,3S,11bS)- and (2R,3R,11bR)-9-(2-Amino-ethoxy)-10-methoxy-3-phenyl-1,3,4,6,7,11b-hexahydro-2H-pyrido[2,1-a]isoquinolin-2-ylamine

This compound was prepared according to the method described in example 14g starting from (2S,3S,11bS)- and (2R,3R,11bR)-(9-hydroxy-10-methoxy-3-phenyl-1,3,4,6,7,11b-hexahydro-2H-pyrido[2,1-a]isoquinolin-2-yl)-carbamic acid tert-butyl ester (190 mg) to yield an orange solid (86 mg).

MS: 368.4 (M+H)⁺

### Example 17

### (2S,3S,11bS)- and (2R,3R11bR)-2-(2-Amino-10-methoxy-3-phenyl-1,3,4,6,7,11b-hexahydro-2H-pyrido[2,1-a]isoquinolin-9-yloxy)-N-methyl-acetamide

### (2S,3S,11bS)- and (2R,3R,11bR)-(10-Methoxy-9-methylcarbamoylmethoxy-3-phenyl-1,3,4,6,7,11b-hexahydro-2H-pyrido[2,1-a]isoquinolin-2-yl)-carbamic acid tert-butyl ester

This compound was synthesized in analogy to example 14f starting from (2S,3S,11bS)- and (2R,3R,11bR)-(9-hydroxy-10-methoxy-3-phenyl-1,3,4,6,7,11b-hexahydro-2H-pyrido[2,1-a]isoquinolin-2-yl)-carbamic acid tert-butyl ester (200 mg) and 2-chloro-N-methyl-acetamide to deliver a yellow solid (160 mg).

MS: 496.3 (M+H)⁺

### (2S,3S,11bS)- and (2R,3R,11bR)-2-(2-Amino-10-methoxy-3-phenyl-1,3,4,6,7,11b-hexahydro-2H-pyrido[2,1-a]isoquinolin-9-yloxy)-N-methyl-acetamide

This compound was synthesized in analogy to example 14g starting from (2S,3S,11bS) and (2R,3R,11bR)-(10-methoxy-9-methylcarbamoylmethoxy-3-phenyl-1,3,4,6,7,11b-hexahydro-2H-pyrido[2,1-a]isoquinolin-2-yl)-carbamic acid tert-butyl ester (150 mg) to yield a light brown solid (96 mg).

MS: 396.3 (M+H)⁺

### Example 18

### (2S,3S,11bS)- and (2R,3R,11bR)-2-(2-Amino-10-methoxy-3-phenyl-1,3,4,6,7,11b-hexahydro-2H-pyrido[2,1-a]isoquinolin-9-yloxy)-N,N-dimethyl-acetamide

### (2S,3S,11bS)- and (2R,3R,11bR)-(9-Dimethylcarbamoylmethoxy-10-methoxy-3-phenyl-1,3,4,6,7,11b-hexahydro-2H-pyrido[2,1-a]isoquinolin-2-yl)-carbamic acid tert-butyl ester

This material was obtained as described in example 14f starting from (2S,3S,11bS) and (2R,3R,11bR)-(9-hydroxy-10-methoxy-3-phenyl-1,3,4,6,7,11b-hexahydro-2H-pyrido[2,1-a]isoquinolin-2-yl)-carbamic acid tert-butyl ester (180 mg) and 2-chloro-N,N-dimethylacetamide to obtain a yellow solid (190 mg).

MS: 510.5 (M+H)⁺

### (2S,3S,11bS) and (2R,3R,11bR)-2-(2-Amino-10-methoxy-3-phenyl-1,3,4,6,7,11b-hexahydro-2H-pyrido[2,1-a]isoquinolin-9-yloxy)-N,N-dimethyl-acetamide

The title compound was obtained as described in example 14g starting from (2S,3S,11bS)- and (2R,3R,11bR)-(9-dimethylcarbamoylmethoxy-10-methoxy-3-phenyl-1,3,4,6,7,11b-hexahydro-2H-pyrido[2,1-a]isoquinolin-2-yl)-carbamic acid tert-butyl ester (180 mg). It was isolated as a yellow solid (59 mg).

MS: 410.6 (M+H)⁺

### Example 19

### (2S,3S,11bS) and (2R,3R,11bR)-2-(2-Amino-10-methoxy-3-phenyl-1,3,4,6,7,11b-hexahydro-2H-pyrido[2,1-a]isoquinolin-9-yloxy)-acetamide

### (2S,3S,11bS)- and (2R,3R,11bR)-(2-tert-Butoxycarbonylamino-10-methoxy-3-phenyl-1,3,4,6,7,11b-hexahydro-2H-pyrido[2,1-a]isoquinolin-9-yloxy)-acetic acid methyl ester

This compound was synthesized according to the procedure described in example 14f starting from (2S,3S,11bS) and (2R,3R,11bR)-(9-hydroxy-10-methoxy-3-phenyl-1,3,4,6,7,11b-hexahydro-2H-pyrido[2,1-a]isoquinolin-2-yl)-carbamic acid tert-butyl ester (300 mg) and methyl bromoacetate to yield a yellow solid (337 mg).

MS: 497.2 (M+H)⁺

### (2S,3S,11bS)- and (2R,3R,11bR)-(9-Carbamoylmethoxy-10-methoxy-3-phenyl-1,3,4,6,7,11b-hexahydro-2H-pyrido[2,1-a]isoquinolin-2-yl)-carbamic acid tert-butyl ester

A suspension of (2S,3S,11bS)- and (2R,3R,11bR)-(2-tert-butoxycarbonylamino-10-methoxy-3-phenyl-1,3,4,6,7,11b-hexahydro-2H-pyrido[2,1-a]isoquinolin-9-yloxy)-acetic acid methyl ester (250 mg) in 5 ml NH₃/MeOH was stirred for 72 h at room temperature, then precipitated from tBuOMe and heptane to deliver the title compound as a yellow solid (106 mg).

MS: 482.6 (M+H)⁺

### (2S,3S,11bS)- and (2R,3R,11bR)-2-(2-Amino-10-methoxy-3-phenyl-1,3,4,6,7,11b-hexahydro-2H-pyrido[2,1-a]isoquinolin-9-yloxy)-acetamide

This compound was prepared according to the procedure described in example 14g starting from (2S,3S,11bS)- and (2R,3R,11bR)-(9-carbamoylmethoxy-10-methoxy-3-phenyl-1,3,4,6,7,11b-hexahydro-2H-pyrido[2,1-a]isoquinolin-2-yl)-carbamic acid tert-butyl ester (96 mg) to provide a yellow solid (50 mg).

MS: 382.3 (M+H)⁺

### Example 20

### (2S,3S,11bS)- and (2R,3R,11bR)-9-Benzyloxy-10-methoxy-3-m-tolyl-1,3,4,6,7,11b-hexahydro-2H-pyrido[2,1-a]isoquinolin-2-ylamine

### (3S,11bS)- and (3R,11bR)-9-Benzyloxy-10-methoxy-3-m-tolyl-1,3,4,6,7,11b-hexahydropyrido[2,1-a]isoquinolin-2-one

This compound was prepared in analogy to example 1a starting from rac-9-benzyloxy-10-methoxy-1,3,4,6,7,11b-hexahydro-pyrido[2,1-a]isoquinolin-2-one(2.05 g) and 3-bromotoluene(1.08 g) to obtain (3S,11bS)- and (3R,11bR)-9-benzyloxy-10-methoxy-3-m-tolyl-1,3,4,6,7,11b-hexahydro-pyrido[2,1-a]isoquinolin-2-one(0.53 g) as a light yellow foam.

MS (ISP): 428.5 (M+H)⁺

### (3S,11bS)- and (3R,11bR)-9-Benzyloxy-10-methoxy-3-m-tolyl-1,3,4,6,7,11b-hexahydropyrido[2,1-a]isoquinolin-2-one oxime

This compound was prepared in analogy to example 1b starting from (3S,11bS)- and (3R,11bR)-9-benzyloxy-10-methoxy-3-m-tolyl-1,3,4,6,7,11b-hexahydro-pyrido[2,1-a]isoquinolin-2-one (0.52 g), hydroxylamine hydrochloride (0.093 g) and sodium acetate (0.11 g) in ethanol (15 mL) to obtain (3S,11bS)- and (3R,11bR)-9-benzyloxy-10-methoxy-3-m-tolyl-1,3,4,6,7,11b-hexahydro-pyrido[2,1-a]isoquinolin-2-one oxime (0.52 g) as an off-white solid.

MS (ISP): 443.4 (M+H)⁺

### (2S,3S,11bS)- and (2R,3R,11bR)-9-Benzyloxy-10-methoxy-3-m-tolyl-1,3,4,6,7,11b-hexahydro-2H-pyrido[2,1-a]isoquinolin-2-ylamine

To a solution of (3S,11bS)- and (3R,11bR)-9-benzyloxy-10-methoxy-3-m-tolyl-1,3,4,6,7,11b-hexahydro-pyrido[2,1-a]isoquinolin-2-one oxime (0.52 g) in ethanol/dioxane (60 mL) was added Raney Ni (3.5 g). Air was removed from the reaction mixture and replaced by hydrogen. Concentrated ammonium hydroxide (2.0 mL) was added by syringe, and the reaction mixture was stirred at room temperature for 3 hours. The suspension was filtered through a microfilter. The filtrate was concentrated, and the residue was chromatographed on silica gel using methylene chloride/methanol/conc. ammonium hydroxide as eluents to obtain (2S,3S,11bS)- and (2R,3R,11bR)-9-benzyloxy-10-methoxy-3-m-tolyl-1,3,4,6,7,11b-hexahydro-2H-pyrido[2,1-a]isoquinolin-2-ylamine (0.21 g) as a light yellow solid. This product was eluted second during chromatography (cf. Example 21).

MS (ISP): 429.4 (M+H)⁺

### Example 21

### (2S,3S,11bS)- and (2R,3R,11bR)-2-Amino-10-methoxy-3-m-tolyl-1,3,4,6,7,11b-hexahydro-2H-pyrido[2,1-a]isoquinolin-9-ol

This compound was prepared in analogy to example 20c starting from (3S,11bS)- and (3R,11bR)-9-benzyloxy-10-methoxy-3-m-tolyl-1,3,4,6,7,11b-hexahydro-pyrido[2,1-a]isoquinolin-2-one oxime (0.52 g). It was obtained as a light red solid (0.182 g). This product was eluted fourth during chromatography. (cf. Example 20c)

MS (ISP): 339.4 (M+H)⁺

### Example 22

### (2S,3S,11bS)- and (2R,3R,11bR)-9-(2-Benzyloxy-ethoxy)-10-methoxy-3-m-tolyl-1,3,4,6,7,11b-hexahydro-2H-pyrido[2,1-a]isoquinolin-2-ylamine

A solution of triphenylphosphine (0.36 g) in abs. THF (10 mL) was cooled to 0° C., diethylazodicarboxylate (0.32 g) was added dropwise over 2 minutes, and the reaction mixture stirred at 0-5° C. for 30 minutes. A mixture of (2S,3S,11bS)- and (2R,3R,11bR)-2-amino-10-methoxy-3-m-tolyl-1,3,4,6,7,11b-hexahydro-2H-pyrido[2,1-a]isoquinolin-9-ol (0.155 g) and benzyloxyethanol (0.28 g) in abs. THF (10 mL) were added in one portion. The reaction mixture was stirred over night at room temperature, concentrated, and the residue was chromatographed on silica gel using methylene chloride/methanol/conc. ammonium hydroxide as the eluent to obtain (2S,3S,11bS)- and (2R,3R,11bR)-9-(2-benzyloxy-ethoxy)-10-methoxy-3-m-tolyl-1,3,4,6,7,11b-hexahydro-2H-pyrido[2,1-a]isoquinolin-2-ylamine (0.18 g) as a light yellow solid.

MS (ISP): 473.4 (M+H)⁺

### Example 23

### (2S,3S,11bS)- and (2R,3R,11bR)-2-(2-Amino-10-methoxy-3-m-tolyl-1,3,4,6,7,11b-hexahydro-2H-pyrido[2,1-a]isoquinolin-9-yloxy)-ethanol

To a solution of (2S,3S,11bS)- and (2R,3R,11bR)-9-(2-benzyloxy-ethoxy)-10-methoxy-3-m-tolyl-1,3,4,6,7,11b-hexahydro-2H-pyrido[2,1-a]isoquinolin-2-ylamine (0.125 g) in dioxane/ethanol 1:1 (12 mL) was added 10% Pd/C (0.05 g) and 1N HCl (0.4 mL). The reaction mixture was hydrogenated at room temperature and 1.1 bar for 2 h and then filtered. The filtrate was concentrated, and the residue was chromatographed on silica gel using methylene chloride/methanol/conc. ammonium hydroxide as the eluent to obtain the title compound (0.075 g) as a light yellow foam.

MS (ISP): 383.3 (M+H)⁺

### Example 24

### (2R,3S,11bS)- and (2S,3R,11bR)-9-Benzyloxy-10-methoxy-3-m-tolyl-1,3,4,6,7,11b-hexahydro-2H-pyrido[2,1-a]isoquinolin-2-ylamine

This product was obtained in the final chromatography described in example 20c eluting as the first compound (0.048 g) as light red crystals.

MS (ISP): 429.4 (M+H)⁺

### Example 25

### (2R,3S,11bS)- and (2S,3R,11bR)-2-Amino-10-methoxy-3-m-tolyl-1,3,4,6,7,11b-hexahydro-2H-pyrido[2,1-a]isoquinolin-9-ol

The title compound was obtained in the final chromatography described in example 20c eluting as third compound (0.019 g) as a red solid.

MS (ISP): 339.3 (M+H)⁺

### Example 26

### ((2S,3S,11bS)- and (2R,3R,11bR)-2-(2-Amino-10-methoxy-3-m-tolyl-1,3,4,6,7,11b-hexahydro-2H-pyrido[2,1-a]isoquinolin-9-yloxy)-acetamide hydrochloride

### (2S,3S,11bS)- and (2R,3R,11bR)-(9-Hydroxy-10-methoxy-3-m-tolyl-1,3,4,6,7,11b-hexahydro-2H-pyrido[2,1-a]isoquinolin-2-yl)-carbamic acid tert-butyl ester

To a solution of (2S,3S,11bS)- and (2R,3R,11bR)-2-Amino-10-methoxy-3-m-tolyl-1,3,4,6,7,11b-hexahydro-2H-pyrido[2,1-a]isoquinolin-9-ol (0.34 g, cf. Ex. 21) in dichloromethane (25 mL) was added di-tert-butyl-dicarbonate (0.24 g). The reaction mixture was stirred under reflux for 2 h, concentrated and chromatographed on silica gel (25 g) using methylene chloride/methanol 19:1 as the eluent to obtain the desired compound (0.43 g) as yellow foam.

MS (ISP): 439.3 (M+H)⁺

### (2S,3S,11bS)- and (2R,3R,11bR)-(2-tert-Butoxycarbonyl-amino-10-methoxy-3-m-tolyl-1,3,4,6,7,11b-hexahydro-2H-pyrido[2,1-a]isoquinolin-9-yloxy)-acetic acid methyl ester

This product was prepared in analogy to example 6a starting from (2S,3S,11bS)- and (2R,3R,11bR)-(9-hydroxy-10-methoxy-3-m-tolyl-1,3,4,6,7,11b-hexahydro-2H-pyrido[2,1-a]isoquinolin-2-yl)-carbamic acid tert-butyl ester (0.40 g), potassium tert-butylate (0.123 g) and methyl bromoacetate (0.167 g) to obtain the desired compound (0.34 g) as colorless crystals.

MS (ISP): 511.5 (M+H)⁺

### (2S,3S,11bS)- and (2R,3R,11bR)-(9-Carbamoylmethoxy-10-methoxy-3-m-tolyl-1,3,4,6,7,11b-hexahydro-2H-pyrido[2,1-a]isoquinolin-2-yl)-carbamic acid tert-butyl ester

This product was prepared in analogy to example 8a starting from (2S,3S,11bS)- and (2R,3R,11bR)-(2-tert-butoxycarbonyl-amino-10-methoxy-3-m-tolyl-1,3,4,6,7,11b-hexahydro-2H-pyrido[2,1-a]isoquinolin-9-yloxy)-acetic acid methyl ester (0.30 g) and 20% NH3/MeOH to obtain the desired compound (0.25 g) as colorless crystals.

MS (ISP): 496.5 (M+H)⁺

### (2S,3S,11bS)- and (2R,3R,11bR)-2-(2-Amino-10-methoxy-3-m-tolyl-1,3,4,6,7,11b-hexahydro-2H-pyrido[2,1-a]isoquinolin-9-yloxy)-acetamide hydrochloride

A suspension of (2S,3S,11bS)- and (2R,3R,11bR)-(9-carbamoylmethoxy-10-methoxy-3-m-tolyl-1,3,4,6,7,11b-hexahydro-2H-pyrido[2,1-a]isoquinolin-2-yl)-carbamic acid tert-butyl ester (0.105 g) in dioxane (5 mL) was treated with 6 M HCl/dioxane (0.5 mL) and stirred at room temperature for 60 hours. Ether (10 mL) was added, the precipitate was filtered, washed with ether and dried to obtain the title compound (0.09 g) as colorless powder.

MS (ISP): 396.5 (M+H)⁺

### Example 27

### (2S,3S,11bS)- and (2R,3R,11bR)-2-(2-Amino-10-methoxy-3-m-tolyl-1,3,4,6,7,11b-hexahydro-2H-pyrido[2,1-a]isoquinolin-9-yloxy)-1-morpholin-4-yl-ethanone hydrochloride

### (2S,3S,11bS)- and (2R,3R,11bR)-[10-Methoxy-9-(2-morpholin-4-yl-2-oxo-ethoxy)-3-m-tolyl-1,3,4,6,7,11b-hexahydro-2H-pyrido[2,1-a]isoquinolin-2-yl]-carbamic acid tert-butyl ester

This product was prepared in analogy to example 26a starting from (2S,3S,11bS)- and (2R,3R,11bR)-(9-hydroxy-10-methoxy-3-m-tolyl-1,3,4,6,7,11b-hexahydro-2H-pyrido[2,1-a]isoquinolin-2-yl)-carbamic acid tert-butyl ester (0.43 g), potassium tert-butylate (0.132 g) and 4-2-(chloroacetyl)morpholine (0.192 g) to obtain after chromatography the desired compound (0.47 g) as colorless crystals.

MS (ISP): 566.5 (M+H)⁺

### (2S,3S,11bS)- and (2R,3R,11bR)-2-(2-Amino-10-methoxy-3-m-tolyl-1,3,4,6,7,11b-hexahydro-2H-pyrido[2,1-a]isoquinolin-9-yloxy)-1-morpholin-4-yl-ethanone hydrochloride

This product was prepared in analogy to example 26d starting from (2S,3S,11bS)- and (2R,3R,11bR)-[10-methoxy-9-(2-morpholin-4-yl-2-oxo-ethoxy)-3-m-tolyl-1,3,4,6,7,11b-hexahydro-2H-pyrido[2,1-a]isoquinolin-2-yl]-carbamic acid tert-butyl ester (0.10 g) and 4N HCl/dioxane (0.5 mL) in dioxane to obtain the title compound (0.085 g) as a colorless powder.

MS (ISP): 466.4 (M+H)⁺

### Example 28

### (2R,3S,11bS)- and (2S,3R,11bR)-9-Benzyloxy-10-methoxy-3-(4-methyl-pyridin-2-yl)-1,3,4,6,7,11b-hexahydro-2H-pyrido[2,1-a]isoquinolin-2-ylamine

This product was prepared in analogy to example 20c starting from (3S,11bS) and (3R,11bR)-9-benzyloxy-10-methoxy-3-(4-methyl-pyridin-2-yl)-1,3,4,6,7,11b-hexahydro-pyrido[2,1-a]isoquinolin-2-one oxime (0.49 g) to obtain the title compound after chromatography (0.064 g) as a yellow foam. This product was eluted first during chromatography.

MS (ISP): 430.5 (M+H)⁺

### Example 29

### (2S,3S,11bS) and (2R,3R,11bR)-2-Amino-10-methoxy-3-(4-methyl-pyridin-2-yl)-1,2,4,6,7,11b-hexahydro-2H-pyrido[2,1-a]isoquinolin-9-ol

This product was prepared in analogy to example 20c from (3S,11bS)- and (3R,11bR)-9-benzyloxy-10-methoxy-3-(4-methyl-pyridin-2-yl)-1,3,4,6,7,11b-hexahydro-pyrido[2,1-a]isoquinolin-2-one oxime (7.2 g) to obtain the title compound (1.90 g) as pink solid. This product was eluted fourth during chromatography (cf. example 28)

MS (ISP): 340.5 (M+H)⁺

### Example 30

### (2R,3S,11bS)- and (2S,3R,11bR)-2-Amino-10-methoxy-3-(4-methyl-pyridin-2-yl)-1,3,4,6,7,11b-hexahydro-2H-pyrido[2,1-a]isoquinolin-9-ol

This product was prepared in analogy to example 20c starting from (3S,11bS)- and (3R,11bR)-9-benzyloxy-10-methoxy-3-(4-methyl-pyridin-2-yl)-1,3,4,6,7,11b-hexahydro-pyrido[2,1-a]isoquinolin-2-one oxime (3.9 g) to obtain the title compound (0.49 g) as an orange foam. This product was eluted third during chromatography (cf. example 28)

MS (ISP): 340.3 (M+H)⁺

### Example 31

### (2S,3S,11bS)- and (2R,3R,11bR)-9-(2-Benzyloxy-ethoxy)-10-methoxy-3-(4-methyl-pyridin-2-yl)-1,3,4,6,7,11b-hexahydro-2H-pyrido[2,1-a]isoquinolin-2-ylamine

This product was prepared in analogy to example 22 starting from (2S,3S,11bS) and (2R,3R,11bR)-2-amino-10-methoxy-3-(4-methyl-pyridin-2-yl)-1,3,4,6,7,11b-hexahydro-2H-pyrido[2,1-a]isoquinolin-9-ol (0.34 g), triphenylphosphine (1.05 g), benzyloxyethanol (0.76 g) and di-tert-butylazodicarboxylate (0.92 g) to obtain the title compound (0.43 g) as an orange foam.

MS (ISP): 474.5 (M+H)⁺

### Example 32

### (2S,3S,11bS)- and (2R,3R,11bR)-2-[2-Amino-10-methoxy-3-(4-methyl-pyridin-2-yl)-1,3,4,6,7,11b-hexahydro-2H-pyrido[2,1-a]isoquinolin-9-yloxy]-ethanol

This product was prepared in analogy to example 23 starting from (2S,3S,11bS) and (2R,3R,11bR)-9-(2-benzyloxy-ethoxy)-10-methoxy-3-(4-methyl-pyridin-2-yl)-1,3,4,6,7,11b-hexahydro-2H-pyrido[2,1-a]isoquinolin-2-ylamine (0.34 g) to obtain the title compound (0.205 g) as a light brownish foam.

MS (ISP): 384.1 (M+H)⁺

### Example 33

### (2S,3S,11bS)- and (2R,3R,11bR))-9-(2-Benzyloxy-1-benzyloxymethyl-ethoxy)-10-methoxy-3-(4-methyl-pyridin-2-yl)-1,3,4,6,7,11b-hexahydro-2H-pyrido[2,1-a]isoquinolin-2-ylamine

This product was prepared in analogy to example 22 starting from (2S,3S,11bS)- and (2R,3R,11bR)-2-amino-10-methoxy-3-(4-methyl-pyridin-2-yl)-1,3,4,6,7,11b-hexahydro-2H-pyrido[2,1-a]isoquinolin-9-ol (0.325 g), triphenylphosphine (1.26 g), 1,3-di-benzyloxy-2-propanol (1.30 g) and diisopropylazodicarboxylate (0.97 g) to obtain the title compound (0.54 g) as an orange foam.

MS (ISP): 594.3 (M+H)⁺

### Example 34

### (2S,3S,11bS)- and (2R,3R,11bR)-2-[2-Amino-10-methoxy-3-(4-methyl-pyridin-2yl)-1,3,4,6,7,11b-hexahydro-2H-pyrido[2,1-a]isoquinolin-9-yloxy]-propane-1,3-diol

This product was prepared in analogy to example 23 starting from (2S,3S,11bS)- and (2R,3R,11bR)-9-(2-benzyloxy-1-benzyloxymethyl-ethoxy)-10-methoxy-3-(4-methyl-pyridin-2-yl)-1,3,4,6,7,11b-hexahydro-2H-pyrido[2,1-a]isoquinolin-2-ylamine (0.30 g) to obtain the title compound (0.18 g) as a brownish foam.

MS (ISP): 414.6 (M+H)⁺

### Example 35

### (S)-3-[(2S,3S,11bS)- and (2R,3R,11bR)-2-Amino-10-methoxy-3-(4-methyl-pyridin-2-yl)-1,3,4,6,7,11b-hexahydro-2H-pyrido[2,1-a]isoquinolin-9-yloxy]-propane-1,2-diol

### (2S,3S,11bS)- and (2R,3R,11bR)-9-((R)-2,2-Dimethyl-[1,3]dioxolan-4-ylmethoxy)-10-methoxy-3-(4-methyl-pyridin-2-yl)-1,3,4,6,7,11b-hexahydro-2H-pyrido[2,1-a]isoquinolin-2-ylamine

This product was prepared in analogy to example 22 starting from (2S,3S,11bS)- and (2R,3R,11bR)-2-amino-10-methoxy-3-(4-methyl-pyridin-2-yl)-1,3,4,6,7,11b-hexahydro-2H-pyrido[2,1-a]isoquinolin-9-ol (0.339 g), triphenylphosphine (1.05 g), [(R)-2,2-dimethyl-[1,3]-dioxolan-4-yl]-methanol (0.66 g) and di-tert-butylazodicarboxylate (0.92 g) to obtain the desired compound (0.345 g) as a light brown foam.

MS (ISP): 454.6 (M+H)⁺

### (S)-3-[(2S,3S,11bS)- and (2R,3R,11bR)-2-Amino-10-methoxy-3-(4-methyl-pyridin-2-yl)-1,3,4,6,7,11b-hexahydro-2H-pyrido[2,1-a]isoquinolin-9-yloxy]-propane-1,2-diol

To a solution of (2S,3S,11bS)- and (2R,3R,11bR)-9-((R)-2,2-dimethyl-[1,3]dioxolan-4-ylmethoxy)-10-methoxy-3-(4-methyl-pyridin-2-yl)-1,3,4,6,7,11b-hexahydro-2H-pyrido[2,1-a]isoquinolin-2-ylamine (0.325 g) in tetrahydrofuran (30 mL) was added 2N HCl (10.0 mL). The reaction mixture was stirred at room temperature for 20 h, then filtered over a column of Amberlite IRA-400. The filtrate was evaporated, and the residue was chromatographed on silica gel using ethyl acetate/methanol/conc. ammonium hydroxide 8:2:0.2 as an eluent to obtain the title compound (0.029 g) as a light brown foam.

MS (ISP): 414.5 (M+H)⁺

### Example 36

### (R)-3-[(2S,3S,11bS)- and (2R,3R,11bR)-2-Amino-10-methoxy-3-(4-methyl-pyridin-2yl)-1,3,4,6,7,11b-hexahydro-2H-pyrido[2,1-a]isoquinolin-9-yloxy]-propane-1,2-diol

### (2S,3S,11bS)- and (2R,3R,11bR)-9-((S)-2,2-Dimethyl-[1,3]dioxolan-4-ylmethoxy)-10-methoxy-3-(4-methyl-pyridin-2-yl)-1,3,4,6,7,11b-hexahydro-2H-pyrido[2,1-a]isoquinolin-2-ylamine

This product was prepared in analogy to Example 35a starting from (2S,3S,11bS)- and (2R,3R,11bR)-2-amino-10-methoxy-3-(4-methyl-pyridin-2-yl)-1,3,4,6,7,11b-hexahydro-2H-pyrido[2,1-a]isoquinolin-9-ol (0.339 g), triphenylphosphine (1.05 g), [(S)-2,2-dimethyl-[1,3]-dioxolan-4-yl]-methanol (0.66 g) and di-tert-butylazodicarboxylate (0.92 g) to obtain the desired compound (0.322 g) as an orange foam.

MS (ISP): 454.8 (M+H)⁺

### (R)-3-(2S,3S,11bS) and (2R,3R,11bR)-3-[2-Amino-10-methoxy-3-(4-methyl-pyridin-2-yl)-1,3,4,6,7,11b-hexahydro-2H-pyrido[2,1-a]isoquinolin-9-yloxy]-propane-1,2-diol

This product was prepared in analogy to example 35b starting from (2S,3S,11bS)- and (2R,3R,11bR)-9-((S)-2,2-dimethyl-[1,3]dioxolan-4-ylmethoxy)-10-methoxy-3-(4methyl-pyridin-2-yl)-1,3,4,6,7,11b-hexahydro-2H-pyrido[2,1-a]isoquinolin-2-ylamine (0.29 g) to obtain after chromatography the title compound (0.042 g) as a light brown foam.

MS (ISP): 414.6 (M+H)⁺

### Example 37

### (2R,3S,11bS)- and (2S,3R,11bR)-2-[2-Amino-10-methoxy-3-(4-methyl-pyridin-2-yl)-1,3,4,6,7,11b-hexahydro-2H-pyrido[2,1-a]isoquinolin-9-yloxy]-1-morpholin-4-yl-ethanone hydrochloride

### (2R,3S,11bS)- and (2S,3R,11bR)-[9-Hydroxy-10-methoxy-3-(4-methyl-pyridin-2yl)-1,3,4,6,7,11b-hexahydro-2H-pyrido[2,1-a]isoquinolin-2-yl]-carbamic acid tert-butyl ester

This product was prepared in analogy to example 2a starting from (2R,3S,11bS)- and (2S,3R,11bR)-2-amino-10-methoxy-3-(4-methyl-pyridin-2-yl)-1,3,4,6,7,11b-hexahydro-2H-pyrido[2,1-a]isoquinolin-9-ol (1.22 g) to obtain the desired compound after chromatography (0.74 g) as a light yellow foam.

MS (ISP): 440.5 (M+H)⁺

### (2R,3S,11bS)- and (2S,3R,11bR)-[10-Methoxy-3-(4-methyl-pyridin-2-yl)-9-(2-morpholin-4-yl-2-oxo-ethoxy)-1,3,4,6,7,11b-hexahydro-2H-pyrido[2,1-a]isoquinolin-2-yl]-carbamic acid tert-butyl ester

This compound was prepared in analogy to example 27a starting from (2R,3S,11bS)- and (2S,3R,11bR)-[9-Hydroxy-10-methoxy-3-(4-methyl-pyridin-2-yl)-1,3,4,6,7,11b-hexahydro-2H-pyrido[2,1-a]isoquinolin-2-yl]-carbamic acid tert-butyl ester (0.445 g) to obtain the desired compound after chromatography (0.17 g) as a light yellow foam.

MS (ISP): 567.5 (M+H)⁺

### (2R,3S,11bS)- and (2S,3R,11bR)-2-[2-Amino-10-methoxy-3-(4-methyl-pyridin-2-yl)-1,3,4,6,7,11b-hexahydro-2H-pyrido[2,1-a]isoquinolin-9-yloxy]-1-morpholin-4-yl-ethanone hydrochloride

This product was prepared in analogy to example 26d starting from (2R,3S,11bS)- and (2S,3R,11bR)-[10-methoxy-3-(4-methyl-pyridin-2-yl)-9-(2-morpholin-4-yl-2-oxo-ethoxy)-1,3,4,6,7,11b-hexahydro-2H-pyrido[2,1-a]isoquinolin-2-yl]-carbamic acid tert-butyl ester (0.15 g) to obtain the title compound (0.132 g) as an amorphous powder.

MS (ISP): 467.1 (M+H)⁺

### Example 38

### (2S,3S,11bS)- and (2R,3R,11bR)-2-[2-Amino-10-methoxy-3-(4-methyl-pyridin-2-yl)-1,3,4,6,7,11b-hexahydro-2H-pyrido[2,1-a]isoquinolin-9-yloxy]-1-morpholin-4-yl-ethanone hydrochloride

### (2S,3S,11bS)- and (2R,3R,11bR)-[9-Hydroxy-10-methoxy-3-(4-methyl-pyridin-2yl)-1,3,4,6,7,11b-hexahydro-2H-pyrido[2,1-a]isoquinolin-2-yl]-carbamic acid tert-butyl ester

This product was prepared in analogy to example 26a starting from (2S,3S,11bS)- and (2R,3R,11bR)-2-amino-10-methoxy-3-(4-methyl-pyridin-2-yl)-1,3,4,6,7,11b-hexahydro-2H-pyrido[2,1-a]isoquinolin-9-ol (0.34 g) and di-tertbutyldicarbonate (0.24 g) to obtain the desired compound (0.405 g) as a light yellow foam.

MS (ISP): 440.4 (M+H)⁺

### (2S,3S,11bS)- and (2R,3R,11bR)-[10-Methoxy-3-(4-methyl-pyridin-2-yl)-9-(2-morpholin-4-yl-2-oxo-ethoxy)-1,3,4,6,7,11b-hexahydro-2H-pyrido[2,1-a]isoquinolin-2-yl]-carbamic acid tert-butyl ester

This product was prepared in analogy to example 27a starting from (2S,3S,11bS)- and (2R,3R,11bR)-[9-hydroxy-10-methoxy-3-(4-methyl-pyridin-2-yl)-1,3,4,6,7,11b-hexahydro-2H-pyri-do[2,1-a]isoquinolin-2-yl]-carbamic acid tert-butyl ester (0.24 g), potassium tert-butylate (0.075 g) and 4-2-chloroacetyl)morpholine (0.107 g) to obtain after chromatography the desired compound (0.267 g) as a colorless foam)

MS (ISP): 567.5 (M+H)⁺

### (2S,3S,11bS)- and (2R,3R,11bR)-2-[2-Amino-10-methoxy-3-(4-methyl-pyridin-2-yl)-1,3,4,6,7,11b-hexahydro-2H-pyrido[2,1-a]isoquinolin-9-yloxy]-1-morpholin-4-yl-ethanone hydrochloride

The title compound was prepared in analogy to example 26d starting from (2S,3S,11bS)- and (2R,3R,11bR)-[10-methoxy-3-(4-methyl-pyridin-2-yl)-9-(2-morpholin-4-yl-2-oxo-ethoxy)-1,3,4,6,7,11b-hexahydro-2H-pyrido[2,1-a]isoquinolin-2-yl]-carbamic acid tert-butyl ester (0.24 g) to obtain the desired compound (0.21 g) as an amorphous powder.

MS (ISP): 467.0 M+H)⁺

### Example 39

### (2S,3S,11bS)- and (2R,3R,11bR)-2-[2-Amino-10-methoxy-3-(4-methyl-pyridin-2-yl)-1,3,4,6,7,11b-hexahydro-2H-pyrido[2,1a]isoquinolin-9-yloxy]-acetamide hydrochloride

### (2S,3S,11bS)- and (2R,3R,11bR)-[9-Carbamoylmethoxy-10-methoxy-3-(4-methyl-pyridin-2-yl)-1,3,4,6,7,11b-hexahy-dro-2H-pyrido[2,1-a]isoquinolin-2-yl]-carbamic acid tert-butyl ester

This product was prepared in analogy to example 8a starting from (2S,3S,11bS)- and (2R,3R,11bR)-[2-tert-Butoxycarbonyl-amino-10-methoxy-3-(4-methyl-pyridin-2-yl)-1,3,4,6,7,11b-hexahydro-2H-pyrido[2,1-a]isoquinolin-9-yloxy]-acetic acid methyl ester (cf. Example 12f) (0.19 g) to obtain the desired compound (0.18 g) as a colorless solid.

MS (ISP): 497.5 (M+H)⁺

### (2S,3S,11bS)- and (2R,3R,11bR)-2-[2-Amino-10-methoxy-3-(4-methyl-pyridin-2-yl)-1,3,4,6,7,11b-hexahydro-2H-pyrido[2,1a]isoquinolin-9-yloxy]-acetamide hydrochloride

The title compound was prepared in analogy to example 26d starting from (2S,3S,11bS) and (2R,3R,11bR)-[9-carbamoylmethoxy-10-methoxy-3-(4-methyl-pyridin-2-yl)-1,3,4,6,7,11b-hexahydro-2H-pyrido[2,1-a]isoquinolin-2-yl]-carbamic acid tert-butyl ester (0.16 g) to obtain the desired product (0.135 g) as a colorless powder.

MS (ISP): 397.3 (M+H)⁺

### Example 40

### (2S,3S,11bS)- and (2R,3R,11bR)-2-Amino-3-(4-fluoromethyl-pyridin-2-yl)-10-methoxy-1,3,4,6,7,11b-hexahydro-2H-pyrido[2,1-a]isoquinolin-9-yloxy]-acetic acid methyl ester

### 2-Bromo-4-(tert-butyl-dimethyl-silanyloxymethyl)-pyridine

To a solution of 2-bromo-4-(hydroxymethyl)pyridine (Lancaster, [CAS 118289-16-0]) (7.3 g) and imidazole (2.65 g) in dichloromethane (80 ml) was added dropwise over 15 minutes at 0-5° C. a solution of tert-butyldimethylsilyl chloride (5.85 g) in dichloro-methane (20 ml). The reaction mixture was stirred at 0-5° C. for 3 h, poured onto ice/water and extracted with dichloromethane. The organic phase was washed with water, sat. sodiumhydrogencarbonate solution and brine, dried over magnesium sulfate and concentrated. The crude compound was filtered over silica gel (200 g) with dichloro-methane as an eluent. The product containing fractions were evaporated to dryness to obtain 2-bromo-4-(tert-butyl-dimethyl-silanyloxymethyl)-pyridine (10.3 g) as a colorless liquid.

### (3R,11bS)- and (3S,11bR)-9-Benzyloxy-3-[4-(tert-butyl-dimethyl-silanyloxymethyl)-pyridin-2-yl]-10-methoxy-1,3,4,6,7,11b-hexahydro-pyrido[2,1-a]isoquinolin-2-one

This compound was synthetized in analogy to example 1a starting from rac-9-benzyloxy-10-methoxy-1,3,4,6,7,11b-hexahydro-pyrido[2,1-a]isoquinolin-2-one (1 g) and 2-bromo-4-(tert-butyl-dimethyl-silanyloxymethyl)-pyridine to yield a yellow oil (606 mg).

MS: 559.5 (M+H)⁺

### Z/E-(3R,11bS)- and (3S,11bR)-9-Benzyloxy-3-[4-(tert-butyl-dimethyl-silanyloxymethyl)-pyridin-2-yl]-10-methoxy-1,3,4,6,7,11b-hexahydro-pyrido[2,1-a]isoquinolin-2-one oxime

Using the same procedure described in example 1b, the title compound was obtained from (3R,11bS) and (3S,11bR)-9-benzyloxy-3-[4-(tert-butyl-dimethyl-silanyloxymethyl)-pyridin-2-yl]-10-methoxy-1,3,4,6,7,11b-hexahydro-pyrido[2,1-a]isoquinolin-2-one (600 mg) after a chromatography (silica gel, AcOEt/MeOH, 19/1) as a yellow foam (469 mg).

MS: 574.5 (M+H)⁺

### Z/E-(3R,11bS)- and (3S,11bR)-3-[4-(tert-Butyl-dimethyl-silanyloxymethyl)-pyridin-2-yl]-9-hydroxy-10-methoxy-1,3,4,6,7,11b-hexahydro-pyrido[2,1-a]isoquinolin-2-one oxime

This compound was synthesized in analogy to example 15c starting from Z/E-(3R,11bS)- and (3S,11bR)-9-benzyloxy-3-[4-(tert-butyl-dimethyl-silanyloxymethyl)-pyridin-2-yl]-10-methoxy-1,3,4,6,7,11b-hexahydro-pyrido[2,1-a]isoquinolin-2-one oxime (400 mg) to obtain a yellow foam (327 mg).

MS: 484.6 (M+H)⁺

### (2S,3S,11bS)- and (2R,3R,11bR)-2-Amino-3-[4-(tert-butyl-dimethyl-silanyloxymethyl)-pyridin-2-yl]-10-methoxy-1,3,4,6,7,11b-hexahydro-2H-pyrido[2,1-a]isoquinolin-9-ol

(2S,3S,11bS)- and (2R,3R,11bR)-2-Amino-3-[4-(tert-butyl-dimethyl-silanyloxymethyl)-pyridin-2-yl]-10-methoxy-1,3,4,6,7,11b-hexahydro-2H-pyrido[2,1-a]isoquinolin-9-ol was prepared according to the procedure described in example 14g starting from Z/E-(3R,11bS)- and (3S,11bR)-3-[4-(tert-butyl-dimethyl-silanyloxymethyl)-pyridin-2-yl]-9-hydroxy-10-methoxy-1,3,4,6,7,11b-hexahydro-pyrido[2,1-a]isoquinolin-2-one oxime (400 mg) to yield a red foam (83 mg).

MS: 470.4 (M+H)⁺

### (2S,3S,11bS)- and (2R,3R,11bR)-{3-[4-(tert-Butyl-dimethyl-silanyloxymethyl)-pyridin-2yl]-9-hydroxy-10-methoxy-1,3,4,6,7,11b-hexahydro-2H-pyrido[2,1-a]isoquinolin-2-yl}-carbamic acid tert-butyl ester

(2S,3S,11bS)- and (2R,3R,11bR)-2-Amino-3-[4-(tert-butyl-dimethyl-silanyloxymethyl)-pyridin-2-yl]-10-methoxy-1,3,4,6,7,11b-hexahydro-2H-pyrido[2,1-a]isoquinolin-9-ol (80 mg) was treated according to the procedure described in example 2a to deliver the title compound as a yellow foam (97 mg).

MS: 570.5 (M+H)⁺

### (2S,3S,11bS)- and (2R,3R,11bR)-[2-tert-Butoxycarbonylamino-3-(4-hydroxymethyl-pyridin-2-yl)-10-methoxy-1,3,4,6,7,11b-hexahydro-2H-pyrido[2,1-a]isoquinolin-9-yloxy]-acetic

(2S,3S,11bS)- and (2R,3R,11bR)-3-[4-(tert-butyl-dimethyl-silanyloxymethyl)-pyridin-2-yl]-9-hydroxy-10-methoxy-1,3,4,6,7,11b-hexahydro-2H-pyrido[2,1-a]isoquinolin-2-yl}-carbamic acid tert-butyl ester (100 mg) was dissolved in DMF. Using the procedure described in example 6a, the title product was obtained as a white solid (68 mg).

MS: 528.3 (M+H)⁺

### (2S,3S,11bS)- and (2R,3R,11bR)-2-Amino-3-(4-fluoromethyl-pyridin-2-yl)-10-methoxy-1,3,4,6,7,11b-hexahydro-2H-pyrido[2,1-a]isoquinolin-9-yloxy]-acetic acid methyl ester

(2S,3S,11bS)- and (2R,3R,11bR)-[2-tert-Butoxycarbonylamino-3-(4-hydroxymethyl-pyridin-2-yl)-10-methoxy-1,3,4,6,7,11b-hexahydro-2H-pyrido[2,1-a]isoquinolin-9-yloxy]-acetic (65 mg) was dissolved in 2 ml dichloromethane under ice cooling. Diethylaminosulfur trifluoride (59 mg) was added and the solution was stirred for 2 hours at 0° C. The reaction mixture was poured on crushed ice/NaHCO₃ and extracted with CH₂Cl₂. The combined organic layers were dried over magnesium sulfate, evaporated and chromatographied (silica gel, CH₂Cl₂/MeOH/NH₄OH, 9/1/0.05). The residue was dissolved in 2 ml dioxane and 0.5 ml 4M HCl/dioxane stirred 4 h at room temperature, precipitated with diethyl ether and filtrated to deliver the title compound as a yellow solid (14 mg).

MS: 431.0 (M+H)⁺

### Example 41

### (2S,3S,11bS)- and (2R,3R,11bR)-9-Benzyloxy-3-(2,5-dimethyl-phenyl)-10-methoxy-1,3,4,6,7,11b-hexahydro-2H-pyrido[2,1-a]isoquinolin-2-ylamine

(3S,11bS)- and (3R,11bR)-9-Benzyloxy-3-(2,5-dimethyl-phenyl)-10-methoxy-1,3,4,6,7,11b-hexahydro-pyrido[2,1-a]isoquinolin-2-one oxime was dissolved in methanol. Using the procedure described in example 1c and reducing the reaction time to 1 h, the title product was obtained as a white solid.

MS: 443.4 (M+H)⁺

### Example 42

### (2S,3S,11bS)- and (2R,3R,11bR)-2-Amino-10-methoxy-3-phenyl-1,3,4,6,7,11b-hexahydro-2H-pyrido[2,1-a]isoquinolin-9-ol

This compound was prepared according to the procedure described in example 1c starting from (3S,11bS)- and (3R,11bR)-9-benzyloxy-10-methoxy-3-phenyl-1,3,4,6,7,11b-hexahydro-pyrido[2,1-a]isoquinolin-2-one oxime. Chromatography (silica gel, CH₂Cl₂/MeOH/NH₄OH, 10/1/0.1) provided an orange solid.

MS: 325.5 (M+H)⁺

### Example 43

### rac-(2S,3S,11bS)-8-Benzyloxy-9-methoxy-3-m-tolyl-1,3,4,6,7,11b-hexahydro-2H-pyrido[2,1-a]isoquinolin-2-ylamine

### N-[2-(2-benzyloxy-3-methoxy-phenyl)-ethyl]-formamide

Carbonyldiimidazole (CDI, 662 mg) was dissolved in THF (15 mL) under nitrogen and a solution of formic acid (0.15 mL) in THF (5 mL) was added slowly over 5 minutes. The resulting mixture was allowed to stir at room temperature for 30 minutes and then a solution of 2-(2-benzyloxy-3-methoxy-phenyl)-ethylamine (1.0 g, made according to Chim. Ther. 1973, 8 (3), 308-313) in THF (10 mL) was added dropwise over a period of 10 minutes. The mixture was stirred and TLC analysis confirmed complete consumption of the starting material after 30 minutes. The reaction mixture was concentrated in vacuo, diluted with dichloromethane (100 mL) washed with aq. HCl solution (1 M, 100 mL) and brine, dried and evaporated to give the crude product as a yellow oil. The residue was purified by flash chromatography (50 g silica gel, gradient of heptane in ethyl acetate (50% to 0%) and the fractions containing the desired product were combined and evaporated to give a colorless oil that solidified upon standing (0.96 g, 87%).

¹H NMR (δ, CDCl₃): 8.00 (s, 1H), 7.45-7.35 (m, 5H), 7.04-6.98 (m, 1H), 6.86 (dd, 1H), 6.77 (dd, 1H), 5.80 (br s, 1H), 5.03 (s, 2H), 3.91 (s, 3H), 3.46 (q, 2H), 2.76 (t, 2H). MS (ESI): 303.2 (MNH₄⁺).

5-Benzyloxy-6-methoxy-3,4-dihydro-isoquinoline, hydrochloride salt and free base

Freshly distilled POCl₃ (2.03 mL) was added to CH₃CN (60 mL) under argon. A solution of N-[2-(2-Benzyloxy-3-methoxy-phenyl)-ethyl]-formamide (2.5 g) in CH₃CN (15 mL) was added by syringe pump over a period of 2 hours and the resulting mixture was allowed to stir for additional 3 hours. Methanol (60 mL) was added carefully and the mixture was stirred for 30 minutes. The solution was concentrated in vacuo and ethyl acetate (50 mL) was added to the residue with stirring. The precipitated product was filtered, washed with a small amount of ethyl acetate and dried in vacuo to give a colorless solid (0.65 g). The filtrate was evaporated and precipitation from ethyl acetate/ether 1:1 gave a second crop of product (0.27 g).

¹H NMR (δ, DMSO-D₆): 13.18 (br s, 1H), 9.01 (s, 1H), 7.76 (d, 1H), 7.44-7.36 (m, 5H), 7.28 (d, 1H), 5.01 (s, 2H), 4.02 (s, 3H), 3.74 (t, 2H), 2.94 (t, 2H). MS (ESI): 268.4 (MH⁺).

Release of free base: 5-benzyloxy-6-methoxy-3,4-dihydro-isoquinoline, hydrochloride salt (5.0 g) was treated with 3N NaOH (200 mL) and the aqueous layer was extracted with ethyl acetate (2×250 mL). The organic layer was washed with brine, dried over MgSO₄, evaporated and dried in vacuo to give 5-benzyloxy-6-methoxy-3,4-dihydro-isoquinoline as a light brown oil (3.12 g).

¹H NMR (δ, CDCl₃): 8.21 (t, 1H), 7.42-7.32 (m, 5H), 7.04 (d, 1H), 6.84 (d, 1H), 4.99 (s, 2H), 3.93 (s, 3H), 3.58 (td, 2H), 2.59 (t, 2H).

### 4-Dimethylamino-3-m-tolyl-butan-2-one hydrochloride salt

3-Methylphenylacetone (1.0 g), dimethylamine hydrochloride (0.825 g) and paraformaldehyde (0.304 g) were added to absolute ethanol (5 mL) and 4 drops of conc. HCl were added. The mixture was heated to reflux for 24 hours, cooled and concentrated in vacuo. Acetone (10 mL) was added to the residue with stirring and the suspension was kept at 0° C. for 1 hour. The solid was filtered and dried in vacuo over night (0.972 g). This material was used without further purification.

¹H NMR (δ, DMSO-D₆): 10.28 (br s, 1H), 7.31 (t, 1H), 7.18-7.10 (m, 3H), 4.51 (dd, 1H), 3.81 (dd, 1H), 3.25-3.10 (m, 1H), 2.71 (s, 6H), 2.31 (s, 3H), 2.07 (s, 3H). MS (ESI): 206.3 (MH⁺).

### rac-(3S,11bS)-8-Benzyloxy-9-methoxy-3-m-tolyl-1,3,4,6,7,11b-hexahydro-pyrido[2,1-a]isoquinolin-2-one

5-Benzyloxy-6-methoxy-3,4-dihydro-isoquinoline (3.65 g) and 4-dimethylamino-3-m-tolyl-butan-2-one hydrochloride (8.24 g) were dissolved in THF (25 mL) and water (25 mL) and the mixture was allowed to stir at room temperature for 36 hours. The mixture was poured into a mixture of ice, sat. NaHCO₃ and brine and extracted with ethyl acetate. The organic layer was washed with brine, dried over Na₂SO₄ and evaporated in vacuo. The crude product was purified by flash chromatography using a gradient of ethyl acetate in hexanes. The fractions containing the desired product were combined and evaporated to yield—after drying in vacuo—rac-(3S,11bS)-8-benzyloxy-9-methoxy-3-m-tolyl-1,3,4,6,7,11b-hexahydro-pyrido[2,1-a]isoquinolin-2-one (4.06 g) as a light yellow foam.

MS (ESI): 428.8 (MH⁺).

### rac-(3S,11bS)-8-Benzyloxy-9-methoxy-3-m-tolyl-1,3,4,6,7,11b-hexahydro-pyrido[2,1-a]isoquinolin-2-one oxime

rac-(3S,11bS)-8-Benzyloxy-9-methoxy-3-m-tolyl-1,3,4,6,7,11b-hexahydro-pyrido[2,1-a]isoquinolin-2-one (0.135 g) was dissolved in abs. methanol (5 mL) and water (2 mL) and ammonium acetate (64 mg) and hydroxylamine hydrochloride (65 mg) were added. The mixture was heated to reflux for 4 hours and the resulting suspension was cooled to room temperature and evaporated. Following addition of water (8 mL) and methanol (0.5 mL), the mixture was filtered and the solid was dried in vacuo over night to yield rac-(3S,11bS)-8-benzyloxy-9-methoxy-3-m-tolyl-1,3,4,6,7,11b-hexahydro-pyrido[2,1-a]isoquinolin-2-one oxime (126 mg) as a white solid.

MS (ESI): 443.5 (MH⁺).

### rac-(2S,3S,11bS)-8-Benzyloxy-9-methoxy-3-m-tolyl-1,3,4,6,7,11b-hexahydro-2H-pyrido[2,1-a]isoquinolin-2-ylamine

rac-(3S,11bS)-8-Benzyloxy-9-methoxy-3-m-tolyl-1,3,4,6,7,11b-hexahydro-pyrido[2,1-a]isoquinolin-2-one oxime (108 mg) was dissolved in methanol (5 mL) and THF (5 mL) and conc. NH₄OH (25%, 1 mL) was added. Raney nickel (500 mg) was added and a H₂-atmosphere was introduced by repeated evacuation/H₂-introduction. The mixture was hydrogenated overnight and—beside a lot of starting material—a new product was observed. The mixture was evaporated and the residue was chromatographed on silica gel using a gradient of methanol in dichloromethane containing 0.5% NH₄OH as an eluent. Beside starting material (84 mg), the desired reduced product rac-(2S,3S,11bS)-8-Benzyloxy-9-methoxy-3-m-tolyl-1,3,4,6,7,11b-hexahydro-2H-pyrido[2,1-a]isoquinolin-2-ylamine was obtained as a light yellow foam (10 mg).

MS (ESI): 429.6 (MH⁺).

### Example 44

### rac-(2S,3S,11bS)-2-Amino-9-methoxy-3-m-tolyl-1,3,4,6,7,11b-hexahydro-2H-pyrido[2,1-a]isoquinolin-8-ol

### rac-(3S,11bS)-8-Hydroxy-9-methoxy-3-m-tolyl-1,3,4,6,7,11b-hexahydro-pyrido[2,1-a]isoquinolin-2-one

rac-(3S,11bS)-8-Benzyloxy-9-methoxy-3-m-tolyl-1,3,4,6,7,11b-hexahydro-pyrido[2,1-a]isoquinolin-2-one (0.486 mg, from example 43d) was dissolved in abs. methanol (7 mL) and abs. THF (7 mL) and 10% Pd on charcoal (85 mg) were added. A H₂-atmosphere was introduced by repeated evacuation/H₂-introduction. Stirring was continued for 18 hours. The reaction mixture was filtered through celite and the filter cake was washed with ethyl acetate. The filtrate was concentrated in vacuo and the residue was purified by flash chromatography to give rac-(3S,11bS)-8-hydroxy-9-methoxy-3-m-tolyl-1,3,4,6,7,11b-hexahydro-pyrido[2,1-a]isoquinolin-2-one (0.148 g) as a light yellow solid.

MS (ESI): 338.1 (MH⁺).

In similar amounts, the side product rac-(3S,11bS)-9-methoxy-3-m-tolyl-1,3,4,6,7,11b-hexahydro-2H-pyrido[2,1-a]isoquinoline-2,8-diol (0.178 g) was isolated as a mixture of diastereomers.

MS (ESI): 340.4 (MH⁺).

### rac-(3S,11bS)-8-Hydroxy-9-methoxy-3-m-tolyl-1,3,4,6,7,11b-hexahydro-pyrido[2,1-a]isoquinolin-2-one oxime

To rac-(3S,11bS)-8-hydroxy-9-methoxy-3-m-tolyl-1,3,4,6,7,11b-hexahydro-pyrido[2,1-a]isoquinolin-2-one (80 mg) dissolved in methanol (4 mL) and water (2 mL) were added ammonium acetate (48 mg) and hydroxylamine hydrochloride (49 mg). The colorless suspension was heated to 60° C. over night, cooled to RT and evaporated in vacuo. Water (8 mL) and methanol (0.5 mL) were added and the suspension was allowed to stir for 45 minutes. The solid was filtered off, washed with water and dried in vacuo. rac-(3S,11bS)-8-hydroxy-9-methoxy-3-m-tolyl-1,3,4,6,7,11b-hexahydro-pyrido[2,1-a]isoquinolin-2-one oxime (72 mg) was obtained as a white solid.

MS (ESI): 353.3 (MH⁺).

### rac-(2S,3S,11bS)-2-Amino-9-methoxy-3-m-tolyl-1,3,4,6,7,11b-hexahydro-2H-pyrido[2,1-a]isoquinolin-8-ol

rac-(3S,11bS)-8-Hydroxy-9-methoxy-3-m-tolyl-1,3,4,6,7,11b-hexahydro-pyrido[2,1-a]isoquinolin-2-one oxime (65 mg) was dissolved in ethanol (3 mL) and dioxane (3 mL) and Raney nickel (1.5 mL of an ethanolic suspension) was added. A H₂-atmosphere was introduced by repeated evacuation/H₂-introduction and then conc. NH₄OH (25% aq. solution, 0.5 mL) was added. The mixture was vigorously stirred at 60° C. for 2 hours and was then filtered through celite. The filter cake was washed well with ethyl acetate and the filtrate was evaporated in vacuo. The residue was purified by flash chromatography on silica gel using a gradient of methanol in dichloromethane (containing 0.5% conc. NH₄OH) as an eluent. The appropriate fractions were combined and evaporated to give the desired product rac-(2S,3S,11bS)-2-amino-9-methoxy-3-m-tolyl-1,3,4,6,7,11b-hexahydro-2H-pyrido[2,1-a]isoquinolin-8-ol (40 mg) as a white solid.

MS (ESI): 339.4 (MH⁺).

### Example 45

### rac-2-(2-Amino-9-methoxy-3-m-tolyl-1,3,46,7,11b-hexahydro-2H-pyrido[2,1-a]isoquinolin-8-yloxy)-acetamide (2S,3S,11bS) and (2R,3S,11bS) diasteromers

### rac-(3S,11bS)-8-Hydroxy-9-methoxy-3-m-tolyl-1,3,4,6,7,11b-hexahydro-pyrido[2,1-a]isoquinolin-2-one-O-benzyl-oxime

rac-(3S,11bS)-8-Hydroxy-9-methoxy-3-m-tolyl-1,3,4,6,7,11b-hexahydro-pyrido[2,1-a]isoquinolin-2-one (0.2 g, from example 44a), O-benzylhydroxylamine (0.365 g) and sodium acetate (0.255 g) were added to ethanol/water 1:1 (12 mL). The resulting suspension was heated to 60° C. for 12 hours. The mixture was poured into ice/sat. NaHCO₃ solution that was saturated with NaCl and the mixture was extracted with ethyl acetate. The organic layer was washed with brine, dried over Na₂SO₄ and evaporated. The residue was purified by flash chromatography on silica gel using a gradient of ethyl acetate in heptane as an eluent. Fractions containing the desired product were combined and evaporated to give a sticky yellow solid that was treated with n-hexane at 0° C. for 60 min. The suspension was filtered, the solid washed with hexane and dried in vacuo to give rac-(3S,11bS)-8-hydroxy-9-methoxy-3-m-tolyl-1,3,4,6,7,11b-hexahydro-pyrido[2,1-a]isoquinolin-2-one-O-benzyl-oxime (0.164 g) as a white solid.

MS (ESI): 443.4 (MH⁺).

### rac-{(3S,11bS)-2-[(E) and/or (Z)-Benzyloxyimino]-9-methoxy-3-m-tolyl-1,3,4,6,7,11b-hexahydro-2H-pyrido[2,1-a]isoquinolin-8-yloxy}-acetic acid methyl ester

rac-(3S,11bS)-8-Hydroxy-9-methoxy-3-m-tolyl-1,3,4,6,7,11b-hexahydro-pyrido[2,1-a]isoquinolin-2-one-O-benzyl-oxime (75 mg) was dissolved in DMF (4 mL) and cooled to 0° C. under argon. Potassium tert-butylate (22 mg) was added in one portion and the mixture was allowed to stir at 0° C. for 30 minutes. Bromoacetic acid methyl ester (0.02 mL) was added dropwise and the mixture was warmed to room temperature and stirred for 2 hours. The reaction mixture was poured into ice/sat. NaHCO₃ solution saturated with NaCl and the mixture was extracted with ethyl acetate. The organic layer was washed with brine, dried over Na₂SO₄ and evaporated. The residue was purified by flash chromatography using a gradient of ethyl acetate in heptane as an eluent. The fractions containing the desired product were combined and evaporated to give—after drying in vacuo—rac-{(3S,11bS)-2-[(E) and/or (Z)-benzyloxyimino]-9-methoxy-3-m-tolyl-1,3,4,6,7,11b-hexahydro-2H-pyrido[2,1-a]isoquinolin-8-yloxy}-acetic acid methyl ester (59 mg) as a light yellow foam.

MS (ESI): 515.5 (MH⁺).

### rac-2-{(3S,11bS)-2-[(E) and/or (Z)-Benzyloxyimino]-9-methoxy-3-m-tolyl-1,3,4,6,7,11b-hexahydro-2H-pyrido[2,1-a]isoquinolin-8-yloxy}-acetamide

rac-{(3S,11bS)-2-[(E) and/or (Z)-Benzyloxyimino]-9-methoxy-3-m-tolyl-1,3,4,6,7,11b-hexahydro-2H-pyrido[2,1-a]isoquinolin-8-yloxy}-acetic acid methyl ester (50 mg) was treated with methanol saturated with NH₃ (3.5 mL) for 3 hours at room temperature. The mixture was evaporated in vacuo to give rac-2-{(3S,11bS)-2-[(E) and/or (Z)-benzyloxyimino]-9-methoxy-3-m-tolyl-1,3,4,6,7,11b-hexahydro-2H-pyrido[2,1-a]isoquinolin-8-yloxy}-acetamide (48 mg) that was used without further purification.

MS (ESI): 500.5 (MH⁺).

### rac-2-(2-Amino-9-methoxy-3-m-tolyl-1,3,4,6,7,11b-hexahydro-2H-pyrido[2,1-a]isoquinolin-8-yloxy)-acetamide, (2S,3S,11bS) and (2R,3S,11bS) diasteromers

rac-2-{(3S,11bS)-2-[(E)-Benzyloxyimino]-9-methoxy-3-m-tolyl-1,3,4,6,7,11b-hexahydro-2H-pyrido[2,1-a]isoquinolin-8-yloxy}-acetamide (45 mg) was dissolved in abs. ethanol (2 mL) and dioxane (2 mL) and Raney nickel (1 mL of an ethanolic suspension) was added. A H₂-atmosphere was introduced by repeated evacuation/H₂-introduction. Conc. NH₄OH (25%, 0.35 mL) was added and the reaction was vigorously stirred at 60° C. for 2 hours. The mixture was filtered through celite and the filter cake was washed well with ethyl acetate. The filtrate was concentrated in vacuo and the residue was purified by flash chromatography to give rac-2-(2-Amino-9-methoxy-3-m-tolyl-1,3,4,6,7,11b-hexahydro-2H-pyrido[2,1-a]isoquinolin-8-yloxy)-acetamide as the (2R,3S,11bS) diasteromer (6 mg); MS (ESI): 396.5 (MH⁺) and the (2S,3S,11bS) diastereomer (23 mg); MS (ESI): 396.5 (MH⁺).

### Example 46

### rac-2-((2S,3S,11bS)-2-Amino-9-methoxy-3-m-tolyl-1,3,4,6,7,11b-hexahydro-2H-pyrido[2,1-a]isoquinolin-8-yloxy)-1-morpholin-4-yl-ethanone

### rac-(3S,11bS)-9-Methoxy-8-(2-morpholin-4-yl-2-oxo-ethoxy)-3-m-tolyl-1,3,4,6,7,11b-hexahydro-pyrido[2,1-a]isoquinolin-2-one O-benzyl-oxime

Morpholine (17 mg, 0.02 mL) was added to toluene (3.5 mL) at room temperature and a solution of trimethylaluminium in toluene (2M, 0.06 mL) was added by syringe. The mixture was allowed to stir for 1 hour and then a solution of rac-{(3S,11bS)-2-[(E)-benzyloxyimino]-9-methoxy-3-m-tolyl-1,3,4,6,7,11b-hexahydro-2H-pyrido[2,1-a]isoquinolin-8-yloxy}-acetic acid methyl ester (50 mg, obtained in example 45b) in toluene (2 mL) was added prior to heating of the mixture at 110° C. TLC confirmed complete consumption of the starting material after 1 hour; stirring was continued at RT over night. The mixture was poured into ice cold water and the aqueous mixture was extracted with ethyl acetate. The organic layer was washed with brine, dried over Na₂SO₄ and evaporated. The residue was purified by flash chromatography using a gradient of methanol in DCM (containing 0.5% conc. NH₄OH) as an eluent. Fractions containing the desired product were combined and evaporated to give rac-(3S,11bS)-9-methoxy-8-(2-morpholin-4-yl-2-oxo-ethoxy)-3-m-tolyl-1,3,4,6,7,11b-hexahydro-pyrido[2,1-a]isoquinolin-2-one O-benzyl-oxime (50 mg) as a yellow foam.

MS (ESI): 570.7 MH⁺).

### rac-2-((2S,3S,11bS)-2-Amino-9-methoxy-3-m-tolyl-1,3,4,6,7,11b-hexahydro-2H-pyrido[2,1-a]isoquinolin-8-yloxy)-1-morpholin-4-yl-ethanone

This compound was obtained in analogy to example 45d by hydrogenation of rac-(3S,11bS)-9-methoxy-8-(2-morpholin-4-yl-2-oxo-ethoxy)-3-m-tolyl-1,3,4,6,7,11b-hexahydro-pyrido[2,1-a]isoquinolin-2-one O-benzyl-oxime (25 mg) to give rac-2-((2S,3S,11bS)-2-amino-9-methoxy-3-m-tolyl-1,3,4,6,7,11b-hexahydro-2H-pyrido[2,1-a]isoquinolin-8-yloxy)-1-morpholin-4-yl-ethanone as a single diasteromer (13 mg).

MS (ESI): 466.6 (MH⁺).

### Example 47

### rac-2-(2-Amino-9-methoxy-3-m-tolyl-1,3,4,6,7,11b-hexahydro-2H-pyrido[2,1-a]isoquinolin-8-yloxy)-1-(4-methyl-piperazin-1-yl)-ethanone, (2S,3S11bS) and (2R,3S,11bS) diasteromers

### rac-(3S,11bS)-9-Methoxy-8-[2-(4-methyl-piperazin-1-yl)-2-oxo-ethoxy]-3-m-tolyl-1,3,4,6,7,11b-hexahydro-pyrido[2,1-a]isoquinolin-2-one-O-benzyl-oxime

This compound was obtained in analogy to example 46a from rac-{(3S, 11bS)-2-[(E) and/or (Z)-benzyloxyimino]-9-methoxy-3-m-tolyl-1,3,4,6,7,11b-hexahydro-2H-pyrido[2,1-a]isoquinolin-8-yloxy}-acetic acid methyl ester (100 mg) and N-methylpiperazine (40 mg) to give rac-(3S,11bS)-9-methoxy-8-[2-(4-methyl-piperazin-1-yl)-2-oxo-ethoxy]-3-m-tolyl-1,3,4,6,7,11b-hexahydro-pyrido[2,1-a]isoquinolin-2-one-O-benzyl-oxime (95 mg).

MS (ESI): 583.5 (MH⁺).

### rac-2-(2-Amino-9-methoxy-3-m-tolyl-1,3,4,6,7,11b-hexahydro-2H-pyrido[2,1-a]isoquinolin-8-yloxy)-1-(4-methyl-piperazin-1-yl)-ethanone, (2S,3S,11bS) and (2R,3S,11bS) diasteromers

This compound was obtained in analogy to example 46b from rac-(3S,11bS)-9-methoxy-8-[2-(4-methyl-piperazin-1-yl)-2-oxo-ethoxy]-3-m-tolyl-1,3,4,6,7,11b-hexahydro-pyrido[2,1-a]isoquinolin-2-one-O-benzyl-oxime (85 mg) by hydrogenation to give rac-2-(2-amino-9-methoxy-3-m-tolyl-1,3,4,6,7,11b-hexahydro-2H-pyrido[2,1-a]isoquinolin-8-yloxy)-1-(4-methyl-piperazin-1-yl)-ethanone as the (2R,3S,11bS)-diastereomer (6 mg); MS (ESI): 479.4 (MH⁺) and the (2S,3S,11bS)-diasteromer (22 mg). MS (ESI): 479.8 (MH⁺).

### Example 48

### rac-2-((2S,3S,11bS)-2-Amino-9-methoxy-3-m-tolyl-1,3,4,6,7,11b-hexahydro-2H-pyrido[2,1-a]isoquinolin-8-yloxy)-N,N-dimethyl-acetamide

### rac-2-{(3S,11bS)-2-[(E) and/or (Z)-Benzyloxyimino]-9-methoxy-3-m-tolyl-1,3,4,6,7,11b-hexahydro-2H-pyrido[2,1-a]isoquinolin-8-yloxy}-N,N-dimethyl-acetamide

This compound was obtained in analogy to example 45b from rac-(3S,11bS)-8-hydroxy-9-methoxy-3-m-tolyl-1,3,4,6,7,11b-hexahydro-pyrido[2,1-a]isoquinolin-2-one-O-benzyl-oxime (0.2 g) by treatment with potassium tert-butylate (58 mg) and 2-chloro-N,N-dimethylacetamide (0.06 mL) to give rac-2-{(3S, 11bS)-2-[(E) and/or (Z)-benzyloxyimino]-9-methoxy-3-m-tolyl-1,3,4,6,7,11b-hexahydro-2H-pyrido[2,1-a]isoquinolin-8-yloxy}-N,N-dimethyl-acetamide (161 mg).

MS (ESI): 528.5 (MH⁺).

### rac-2-((2S,3S,11bS)-2-Amino-9-methoxy-3-m-tolyl-1,3,4,6,7,11b-hexahydro-2H-pyrido[2,1-a]isoquinolin-8-yloxy)-N,N-dimethyl-acetamide

This compound was obtained in analogy to example 45d from rac-2-{(3S,11bS)-2-[(E) and/or (Z)-benzyloxyimino]-9-methoxy-3-m-tolyl-1,3,4,6,7,11b-hexahydro-2H-pyrido[2,1-a]isoquinolin-8-yloxy}-N,N-dimethyl-acetamide (156 mg) by hydrogenation to give rac-2-((2S,3S,11bS)-2-amino-9-methoxy-3-m-tolyl-1,3,4,6,7,11b-hexahydro-2H-pyrido[2,1-a]isoquinolin-8-yloxy)-N,N-dimethyl-acetamide (82 mg) as a single diasteromer.

MS (ESI): 424.5 MH⁺.

### Example 49

### rac-9-Methoxy-8-(2-methoxy-ethoxy)-3-m-tolyl-1,3,4,6,7,11b-hexahydro-2H-pyrido[2,1-a]isoquinolin-2-ylamine, (2S,3S,11bS) and (2R,3S,11bS) diasteromers

### rac-(3S, 11bS)-9-Methoxy-8-(2-methoxy-ethoxy)-3-m-tolyl-1,3,4,6,7,11b-hexahydro-pyrido[2,1-a]isoquinolin-2-one-O-benzyl-oxime

This compound was obtained as described in example 45b from rac-(3S,11bS)-8-hydroxy-9-methoxy-3-m-tolyl-1,3,4,6,7,11b-hexahydro-pyrido[2,1-a]isoquinolin-2-one-O-benzyl-oxime (200 mg) by treatment with potassium tert-butylate (90 mg) and 2-chloroethyl-methylether (0.09 mL) in DMF (6 mL) to give rac-(3S,11bS)-9-methoxy-8-(2-methoxy-ethoxy)-3-m-tolyl-1,3,4,6,7,11b-hexahydro-pyrido[2,1-a]isoquinolin-2-one-O-benzyl-oxime (88 mg) as a yellow gum.

MS (ESI): 501.5 MH⁺).

### rac-9-Methoxy-8-(2-methoxy-ethoxy)-3-m-tolyl-1,3,4,6,7,11b-hexahydro-2H-pyrido[2,1-a]isoquinolin-2-ylamine, (2S,3S,11bS) and (2R,3S,11bS) diastereomers

This compound was obtained from rac-(3S,11bS)-9-methoxy-8-(2-methoxy-ethoxy)-3-m-tolyl-1,3,4,6,7,11b-hexahydro-pyrido[2,1-a]isoquinolin-2-one O-benzyl-oxime (81 mg) by hydrogenation as described in example 45d to give rac-9-methoxy-8-(2-methoxy-ethoxy)-3-m-tolyl-1,3,4,6,7,11b-hexahydro-2H-pyrido[2,1-a]isoquinolin-2-ylamine as the (2R,3S,11bS) diastereomer (6 mg, not characterized), and the (2S,3S,11bS) diastereomer (35 mg); MS (ESI): 397.4 MH⁺.

### Example 50

### rac-2-(2-Amino-9-methoxy-3-m-tolyl-1,3,4,6,7,11b-hexahydro-2H-pyrido[2,1-a]isoquinolin-8-yloxy)-ethanol, (2S,3S, 11bS) and (2R,3S,11bS) diasteromers

### rac-(3S,11bS)-8-(2-Benzyloxy-ethoxy)-9-methoxy-3-m-tolyl-1,3,4,6,7,11b-hexahydro-pyrido[2,1-a]isoquinolin-2-one-O-benzyl-oxime

This compound was obtained as described in example 45b from rac-(3S,11bS)-8-hydroxy-9-methoxy-3-m-tolyl-1,3,4,6,7,11b-hexahydro-pyrido[2,1-a]isoquinolin-2-one O-benzyl-oxime (105 mg) by treatment with potassium tert-butylate (31 mg) and 2-bromoethyl-benzylether (0.05 mL) in DMF (4 mL) to give rac-(3S,11bS)-8-(2-benzyloxy-ethoxy)-9-methoxy-3-m-tolyl-1,3,4,6,7,11b-hexahydro-pyrido[2,1-a]isoquinolin-2-one-O-benzyl-oxime (113 mg) as a yellow gum.

MS (ESI): 577.4 (MH⁺).

### rac-2-(2-Amino-9-methoxy-3-m-tolyl-1,3,4,6,7,11b-hexahydro-2H-pyrido[2,1-a]isoquinolin-8-yloxy)-ethanol, (2S,3S,11bS) and (2R,3S,11bS) diasteromers

This compound was obtained from rac-(3S,11bS)-8-(2-benzyloxy-ethoxy)-9-methoxy-3-m-tolyl-1,3,4,6,7,11b-hexahydro-pyrido[2,1-a]isoquinolin-2-one-O-benzyl-oxime (105 mg) by hydrogenation as described in example 45d to give rac-2-(2-amino-9-methoxy-3-m-tolyl-1,3,4,6,7,11b-hexahydro-2H-pyrido[2,1-a]isoquinolin-8-yloxy)-ethanol as the (2R,3S,11bS)-diastereomer (8 mg); MS (ESI): 383.3 (MH⁺) and the (2S,3S,11bS)-diastereomer (43 mg). MS (ESI): 383.3 (MH⁺).

### Example 51

Film coated tablets containing the following ingredients can be manufactured in a conventional manner:

| Ingredients | Per tablet | | | |
|---|---|---|---|---|
| Kernel: | | | | |
| Compound of formula (I) | 10.0 | mg | 200.0 | mg |
| Microcrystalline cellulose | 23.5 | mg | 43.5 | mg |
| Lactose hydrous | 60.0 | mg | 70.0 | mg |
| Povidone K30 | 12.5 | mg | 15.0 | mg |
| Sodium starch glycolate | 12.5 | mg | 17.0 | mg |
| Magnesium stearate | 1.5 | mg | 4.5 | mg |
| (Kernel Weight) | 120.0 | mg | 350.0 | mg |
| Film Coat: | | | | |
| Hydroxypropyl methyl cellulose | 3.5 | mg | 7.0 | mg |
| Polyethylene glycol 6000 | 0.8 | mg | 1.6 | mg |
| Talc | 1.3 | mg | 2.6 | mg |
| Iron oxide (yellow) | 0.8 | mg | 1.6 | mg |
| Titanium dioxide | 0.8 | mg | 1.6 | mg |

The active ingredient is sieved and mixed with microcrystalline cellulose and the mixture is granulated with a solution of polyvinylpyrrolidone in water. The granulate is mixed with sodium starch glycolate and magnesium stearate and compressed to yield kernels of 120 or 350 mg respectively. The kernels are lacquered with an aq. solution/suspension of the above mentioned film coat.

### Example 52

Capsules containing the following ingredients can be manufactured in a conventional manner:

| Ingredients | Per capsule |
|---|---|
| Compound of formula (I) | 25.0 mg |
| Lactose | 150.0 mg |
| Maize starch | 20.0 mg |
| Talc | 5.0 mg |

The components are sieved and mixed and filled into capsules of size 2.

### Example 53

Injection solutions can have the following composition:

| Ingredients | |
|---|---|
| Compound of formula (I) | 3.0 mg |
| Polyethylene Glycol 400 | 150.0 mg |
| Acetic Acid | q.s. ad pH 5.0 |
| Water for injection solutions | ad 1.0 ml |

The active ingredient is dissolved in a mixture of polyethylene glycol 400 and water for injection (part). The pH is adjusted to 5.0 by acetic acid. The volume is adjusted to 1.0 ml by addition of the residual amount of water. The solution is filtered, filled into vials using an appropriate overage and sterilized.

### Example 54

Soft gelatin capsules containing the following ingredients can be manufactured in a conventional manner:

| Ingredients | | |
|---|---|---|
| Capsule contents | | |
| Compound of formula (I) | 5.0 | mg |
| Yellow wax | 8.0 | mg |
| Hydrogenated Soya bean oil | 8.0 | mg |
| Partially hydrogenated plant oils | 34.0 | mg |
| Soya bean oil | 110.0 | mg |
| Weight of capsule contents | 165.0 | mg |
| Gelatin capsule | | |
| Gelatin | 75.0 | mg |
| Glycerol 85% | 32.0 | mg |
| Karion 83 | 8.0 | mg (dry matter) |
| Titanium dioxide | 0.4 | mg |
| Iron oxide yellow | 1.1 | mg |

The active ingredient is dissolved in a warm melting of the other ingredients and the mixture is filled into soft gelatin capsules of appropriate size. The filled soft gelatin capsules are treated according to the usual procedures.

### Example 55

Sachets containing the following ingredients can be manufactured in a conventional manner:

| Ingredients | | |
|---|---|---|
| Compound of formula (I) | 50.0 | mg |
| Lactose, fine powder | 1015.0 | mg |
| Microcrystalline cellulose (AVICEL PH 102) | 1400.0 | mg |
| Sodium carboxymethyl cellulose | 14.0 | mg |
| Polyvinylpyrrolidone K 30 | 10.0 | mg |
| Magnesium stearate | 10.0 | mg |
| Flavoring additives | 1.0 | mg |

The active ingredient is mixed with lactose, microcrystalline cellulose and sodium carboxymethyl cellulose and granulated with a mixture of polyvinylpyrrolidone in water. The granulate is mixed with magnesium stearate and the flavoring additives and filled into sachets.

It is to be understood that the invention is not limited to the particular embodiments of the invention described above, as variations of the particular embodiments may be made and still fall within the scope of the appended claims.

What is claimed is:

1. A compound of the formula (I): 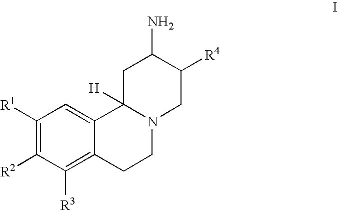
wherein
R¹ is selected from hydrogen or methoxy;
R² is selected from the group consisting of
hydroxy,
lower alkoxy, provided that R² is not methoxy in case R¹ is methoxy,
lower alkoxy mono- or disubstituted by
hydroxy, lower alkoxy, benzyloxy,
amino, alkylamino, dialkylamino, cyano,
unsubstituted phenyl, phenyl substituted by one to three groups selected from lower alkyl, lower alkoxy, halogen and lower halogenalkyl, or
tetrazolyl,
—O—(CH₂)ₘ—C(O)—NR⁸R⁹, wherein m is 1 or 2 and wherein R⁸ and R⁹ are independently selected from hydrogen, lower alkyl or tetrazolyl, or R⁸ and R⁹ together with the nitrogen atom to which they are attached form a 5- or 6-membered heterocycle which may contain an additional heteroatom selected from N, O or S, and which may be substituted by lower alkyl,
—O—(CH₂)ₙ—COOR¹⁰, wherein n is 1 or 2 and R¹⁰ is hydrogen or lower alkyl,
—O—(CH₂)ₚ—NH—C(O)—OR¹¹, wherein p is 1 or 2 and wherein R¹¹ is lower alkyl,
—O—SO₂—R¹², wherein R¹² is lower alkyl,
—NR¹³R¹⁴, wherein R¹³ is hydrogen or lower alkyl and R¹⁴ is lower alkyl or benzyl, and —NH—CO—(CH₂)_{q}—R¹⁵, wherein q is 1 or 2 and wherein R¹⁵ is lower alkyl or tetrazolyl;
R³ is selected from the group consisting of
hydrogen,
hydroxy,
lower alkoxy,
lower alkoxy mono- or disubstituted by
hydroxy, alkoxy, benzyloxy,
amino, alkylamino, dialkylamino, cyano,
unsubstituted phenyl, phenyl substituted one to three groups selected from lower alkyl,
lower alkoxy, halogen and lower halogenalkyl, or
tetrazolyl,
—O—(CH₂)ₘ—C(O)—NR⁸R⁹, wherein m is 1 or 2 and wherein R⁸ and R⁹ are independently selected from hydrogen, lower alkyl or tetrazolyl, or R⁸ and R⁹ together with the nitrogen atom to which they are attached form a 5- or 6-membered heterocycle which may contain an additional heteroatom selected from N, O or S, and which may be substituted by lower alkyl,
—O—(CH₂)ₙ—COOR¹⁰, wherein n is 1 or 2 and R¹⁰ is hydrogen or lower alkyl,
—O—(CH₂)ₚ—NH—C(O)—OR¹¹, wherein p is 1 or 2 and wherein R¹¹ is lower alkyl,
—O—SO₂—R¹², wherein R¹² is lower alkyl,
—NR¹³R¹⁴, wherein R¹³ is hydrogen or lower alkyl and R¹⁴ is lower alkyl or benzyl, and
—NH—CO—(CH₂)_{q}—R¹⁵, wherein q is 1 or 2 and wherein R¹⁵ is lower alkyl or tetrazolyl;
R⁴ is 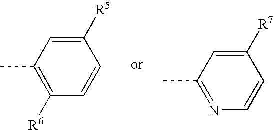
wherein
R⁵ is selected from the group consisting of lower alkyl, lower hydroxyalkyl, lower halogenalkyl, halogen and cycloalkyl; or
R⁵ can also be hydrogen in case R² is selected from the group consisting of —(CH₂)ₘ—C(O)—NR⁸R⁹, —O—(CH₂)ₚ—NH—C(O)—OR¹¹, —O—SO₂—R¹², —NR¹³R¹⁴,
—NH—CO—(CH₂)_{q}—R¹⁵ and lower alkoxy which is mono- or disubstituted by a group selected from hydroxy, benzyloxy, amino, alkylamino, dialkylamino or cyano;
R⁶ is selected from the group consisting of hydrogen, lower alkyl, lower alkoxy, lower hydroxyalkyl, lower halogenalkyl, halogen and cycloalkyl;
R⁷ is selected from the group consisting of lower alkyl, cycloalkyl, lower hydroxyalkyl, halogen and lower halogenalkyl;
and pharmaceutically acceptable salts thereof.

2. The compound according to claim 1, wherein
R⁴ is 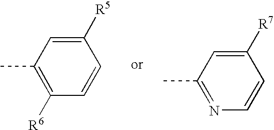
and wherein
R⁵ is selected from the group consisting of lower alkyl, lower hydroxyalkyl, lower halogenalkyl, halogen and cycloalkyl;
R⁶ is selected from the group consisting of hydrogen, lower alkyl, lower alkoxy, lower hydroxyalkyl, lower halogenalkyl, halogen and cycloalkyl; and
R⁷ is selected from the group consisting of lower alkyl, cycloalkyl, lower hydroxyalkyl, halogen and lower halogenalkyl.

3. The compound according to claim 1, wherein R² is selected from the group consisting of hydroxy,
lower alkoxy, provided that R² is not methoxy in case R¹ is methoxy,
lower alkoxy mono- or disubstituted by
hydroxy, lower alkoxy, benzyloxy,
amino, alkylamino, dialkylamino, cyano,
unsubstituted phenyl, phenyl substituted by one to three groups selected from lower alkyl, lower alkoxy, halogen and lower halogenalkyl, or
tetrazolyl,
—O—(CH₂)ₘ—C(O)—NR⁸R⁹, wherein m is 1 or 2 and wherein R⁸ and R⁹ are independently selected from hydrogen, lower alkyl or tetrazolyl, or R⁸ and R⁹ together with the nitrogen atom to which they are attached form a 5- or 6-membered heterocycle which may contain an additional heteroatom selected from N, O or S, and which may be substituted by lower alkyl,
—O—(CH₂)ₙ—COOR¹⁰, wherein n is 1 or 2 and R¹⁰ is hydrogen or lower alkyl,
—O—(CH₂)ₚ—NH—C(O)—OR¹¹, wherein p is 1 or 2 and wherein R¹¹ is lower alkyl,
—O—SO₂—R¹², wherein R¹² is lower alkyl,
—NR¹³R¹⁴, wherein R¹³ is hydrogen or lower alkyl and R¹⁴ is lower alkyl or benzyl, and
—NH—CO—(CH₂)_{q}—R¹⁵, wherein q is 1 or 2 and wherein R¹⁵ is lower alkyl or tetrazolyl;
and R³ is selected from the group consisting of hydrogen, hydroxy and lower alkoxy.

4. The compound according to claim 1, wherein R² is selected from the group consisting of hydroxy,
lower alkoxy mono- or disubstituted by hydroxy, benzyloxy, amino, cyano, phenyl or tetrazolyl,
—O—(CH₂)ₘ—C(O)—NR⁸R⁹, wherein m is 1 or 2 and wherein R⁸ and R⁹ are independently selected from hydrogen, lower alkyl or tetrazolyl, or R⁸ and R⁹ together with the nitrogen atom to which they are attached form a 5- or 6-membered heterocycle which may contain an additional heteroatom selected from N, O or S, and which may be substituted by lower alkyl,
—O—(CH₂)ₙ—COOR¹⁰, wherein n is 1 or 2 and R¹⁰ is hydrogen or lower alkyl,
—O—(CH₂)ₚ—NH—C(O)—OR¹¹, wherein p is 1 or 2 and wherein R¹¹ is lower alkyl,
—O—SO₂—R¹², wherein R¹² is lower alkyl,
—NR¹³R¹⁴, wherein R¹³ is hydrogen or lower alkyl and R¹⁴ is lower alkyl or benzyl, and
—NH—CO—(CH₂)_{q}—R¹⁵, wherein q is 1 or 2 and wherein R¹⁵ is lower alkyl or tetrazolyl.

5. The compound according to claim 1, wherein R² is hydroxy or lower alkoxy mono- or disubstituted by hydroxy, benzyloxy, amino, cyano, phenyl or tetrazolyl.

6. The compound according to claim 1, wherein R² is
—O—(CH₂)ₘ—C(O)—NR⁸R⁹, wherein m is 1 or 2 and wherein R⁸ and R⁹ are independently selected from hydrogen, lower alkyl or tetrazolyl, or R⁸ and R⁹ together with the nitrogen atom to which they are attached form a 5- or 6-membered heterocycle which may contain an additional heteroatom selected from N, O or S, and which may be substituted by lower alkyl.

7. The compound according to claim 6, wherein R² is
—O—(CH₂)ₘ—C(O)—NR⁸R⁹, wherein m is 1 or 2 and wherein R⁸ and R⁹ are independently selected from hydrogen, lower alkyl or tetrazolyl.

8. The compound according to claim 1, wherein R² is
—O—(CH₂)ₙ—COOR¹⁰, wherein n is 1 or 2 and R¹⁰ is hydrogen or lower alkyl.

9. The compound according to claim 1, wherein R² is
—O—SO₂—R¹², wherein R¹² is lower alkyl.

10. The compound according to claim 1, wherein R² is
—NH—CO—(CH₂)_{q}—R¹⁵, wherein q is 1 or 2 and wherein R¹⁵ is lower alkyl or tetrazolyl.

11. The compound according to claim 1, wherein R³ is hydrogen.

12. The compound according to claim 1, wherein R³ is selected from the group consisting of hydroxy,
lower alkoxy,
lower alkoxy mono- or disubstituted by
hydroxy, alkoxy, benzyloxy,
amino, alkylamino, dialkylamino, cyano,
unsubstituted phenyl, phenyl substituted one to three groups selected from lower alkyl, lower alkoxy, halogen and lower halogenalkyl, or
tetrazolyl, and
—O—(CH₂)ₘ—C(O)—NR⁸R⁹, wherein m is 1 or 2 and wherein R⁸ and R⁹ are independently selected from hydrogen, lower alkyl or tetrazolyl, or R⁸ and R⁹ together with the nitrogen atom to which they are attached form a 5- or 6-membered heterocycle which may contain an additional heteroatom selected from N, O or S, and which may be substituted by lower alkyl, and R² is hydroxy or lower alkoxy.

13. The compound according to claim 1, wherein R³ is hydroxy or lower alkoxy mono- or disubstituted by hydroxy, alkoxy, benzyloxy or phenyl.

14. The compound according to claim 1, wherein R³ is —O—(CH₂)ₘ—C(O)—NR⁸R⁹, wherein m is 1 or 2 and wherein R⁸ and R⁹ are independently selected from hydrogen, lower alkyl or tetrazolyl, or R⁸ and R⁹ together with the nitrogen atom to which they are attached form a 5- or 6-membered heterocycle which may contain an additional heteroatom selected from N, O or S, and which may be substituted by lower alkyl.

15. The compound according to claim 1, wherein R² is methoxy.

16. The compound according to claim 1, wherein R⁴ is 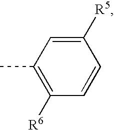
R⁵ is selected from the group consisting of lower alkyl, lower hydroxyalkyl, lower halogenalkyl, halogen and cycloalkyl; and
R⁶ is selected from the group consisting of hydrogen, lower alkyl, lower alkoxy, lower hydroxyalkyl, lower halogenalkyl, halogen and cycloalkyl.

17. The compound according to claim 1, wherein R⁵ is lower alkyl or lower halogenalkyl, and R⁶ is hydrogen or lower alkyl.

18. The compound according to claim 1, wherein R⁴ is 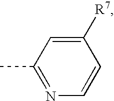
and R⁷ is lower alkyl, cycloalkyl, lower hydroxyalkyl, halogen or lower halogenalkyl.

19. The compound according to claim 1, wherein R⁷ is lower alkyl.

20. The compound according to claim 1, selected from the group consisting of:
(2S,3S,11bS)- and (2R,3R,11bR)-9-(2-amino-ethoxy)-3-(2,5-dimethyl-phenyl)-10-methoxy-1,3,4,6,7,11b-hexahydro-2H-pyrido[2,1-a]isoquinolin-2-ylamine,
(2S,3S,11bS)- and (2R,3R,11bR)-2-amino-3-(2,5-dimethyl-phenyl)-10-methoxy-1,3,4,6,7,11b-hexahydro-2H-pyrido[2,1-a]isoquinolin-9-ol,
(2S,3S,11bS)- and (2R,3R,11bR)-3-(2,5-dimethyl-phenyl)-10-methoxy-9-[2-(1H-tetrazol-5-yl)-ethoxy]-1,3,4,6,7,11b-hexahydro-2H-pyrido[2,1-a]isoquinolin-2-ylamine hydrochloride,
(2S,3S,11bS)- and (2R,3R,11bR)-3-[2-amino-3-(2,5-dimethyl-phenyl)-10-methoxy-1,3,4,6,7,11b-hexahydro-2H-pyrido[2,1-a]isoquinolin-9-yloxy]-propionitrile,
(2S,3S,11bS)- and (2R,3R,11bR)-methanesulfonic acid 2-amino-3-(2,5-dimethyl-phenyl)-10-methoxy-1,3,4,6,7,11b-hexahydro-2H-pyrido[2,1-a]isoquinolin-9-yl ester hydrochloride,
(2S,3S,11bS)- and (2R,3R,11bR)-2-[2-amino-3-(2,5-dimethyl-phenyl)-10-methoxy-1,3,4,6,7,11b-hexahydro-2H-pyrido[2,1-a]isoquinolin-9-yloxy]-ethanol hydrochloride
(2S,3S,11bS)- and (2R,3R,11bR)-[2-amino-3-(2,5-dimethyl-phenyl)-10-methoxy-1,3,4,6,7,11b-hexahydro-2H-pyrido[2,1-a]isoquinolin-9-yloxy]-acetic acid hydrochloride,
(2S,3S,11bS)- and (2R,3R,11bR)-2-[2-amino-3-(2,5-dimethyl-phenyl)-10-methoxy-1,3,4,6,7,11b-hexahydro-2H-pyrido[2,1-a]isoquinolin-9-yloxy]-N-(1 H-tetrazol-5-yl)-acetamide hydrochloride,
(2S,3S,11bS)- and (2R,3R,11bR)-2-[2-amino-3-(2,5-dimethyl-phenyl)-10-methoxy-1,3,4,6,7,11b-hexahydro-2H-pyrido[2,1-a]isoquinolin-9-yloxy]-acetamide hydrochloride,
(2S,3S,11bS)- and (2R,3R,11bR)-2-[2-amino-3-(2,5-dimethyl-phenyl)-10-methoxy-1,3,4,6,7,11b-hexahydro-2H-pyrido[2,1-a]isoquinolin-9-yloxy]-N-methyl-acetamide hydrochloride,
(2S,3S,11bS)- and (2R,3R,11bR)-N-[2-Amino-10-methoxy-3-(4-methyl-pyridin-2-yl)-1,3,4,6,7,11b-hexahydro-2H-pyrido[2,1-a]isoquinolin-9-yl]-2-(1H-tetrazol-5-yl)-acetamide hydrochloride,
(2S,3S,11bS)- and (2R,3R,11bR)-10-methoxy-9-methylamino-3-(4-methyl-pyridin-2-yl)-1,3,4,6,7,11b-hexahydro-2H-pyrido[2,1-a]isoquinoline-2,9-diamine hydrochloride,
(2S,3S,11bS)- and (2R,3R,11bR)-[2-amino-10-methoxy-3-(4-methyl-pyridin-2-yl)-1,3,4,6,7,11b-hexahydro-2H-pyrido[2,1-a]isoquinolin-9-yloxy]-acetic acid hydrochloride,
(2S,3S,11bS)- and (2R,3R,11bR)-2-[2-amino-10-methoxy-3-(4-methyl-pyridin-2-yl)-1,3,4,6,7,11b-hexahydro-2H-pyrido[2,1-a]isoquinolin-9-yloxy]-N-(2H-tetrazol-5-yl)-acetamide hydrochloride,
(2S,3S,11bS)- and (2R,3R,11bR)-methanesulfonic acid 2-amino-10-methoxy-3-phenyl-1,3,4,6,7,11b-hexahydro-2H-pyrido[2,1-a]isoquinolin-9-yl ester,
(2S,3S,11bS)- and (2R,3R,11bR)-2-(2-amino-10-methoxy-3-phenyl-1,3,4,6,7,11b-hexahydro-2H-pyrido[2,1-a]isoquinolin-9-yloxy)-N-methyl-acetamide,
(2S,3S,11bS)- and (2R,3R,11bR)-2-(2-amino-10-methoxy-3-phenyl-1,3,4,6,7,11b-hexahydro-2H-pyrido[2,1-a]isoquinolin-9-yloxy)-N,N-dimethyl-acetamide,
(2S,3S,11bS)- and (2R,3R,11bR)-2-amino-10-methoxy-3-m-tolyl-1,3,4,6,7,11b-hexahydro-2H-pyrido[2,1-a]isoquinolin-9-ol,
(2S,3S,11bS)- and (2R,3R,11bR)-2-(2-amino-10-methoxy-3-m-tolyl-1,3,4,6,7,11b-hexahydro-2H-pyrido[2,1-a]isoquinolin-9-yloxy)-ethanol,
(2S,3S,11bS)- and (2R,3R,11bR)-2-(2-amino-10-methoxy-3-m-tolyl-1,3,4,6,7,11b-hexahydro-2H-pyrido[2,1-a]isoquinolin-9-yloxy)-acetamide hydrochloride,
(2S,3S,11bS)- and (2R,3R,11bR)-2-(2-amino-10-methoxy-3-m-tolyl-1,3,4,6,7,11b-hexahydro-2H-pyrido[2,1-a]isoquinolin-9-yloxy)-1-morpholin-4-yl-ethanone hydrochloride,
(2S,3S,11bS) and (2R,3R,11bR)-2-amino-10-methoxy-3-(4-methyl-pyridin-2-yl)-1,3,4,6,7,11b-hexahydro-2H-pyrido[2,1-a]isoquinolin-9-ol,
(2S,3S,11bS)- and (2R,3R,11bR)-2-[2-amino-10-methoxy-3-(4-methyl-pyridin-2-yl)-1,3,4,6,7,11b-hexahydro-2H-pyrido[2,1-a]isoquinolin-9-yloxy]-ethanol,
(2S,3S,11bS)- and (2R,3R,11bR)-2-[2-amino-10-methoxy-3-(4-methyl-pyridin-2-yl)-1,3,4,6,7,11b-hexahydro-2H-pyrido[2,1-a]isoquinolin-9-yloxy]-propane-1,3-diol,
(2S,3S,11bS)- and (2R,3R,11bR)-2-[2-amino-10-methoxy-3-(4-methyl-pyridin-2-yl)-1,3,4,6,7,11b-hexahydro-2H-pyrido[2,1-a]isoquinolin-9-yloxy]-1-morpholin-4-yl-ethanone hydrochloride,
(2S,3S,11bS)- and (2R,3R,11bR)-2-[2-amino-10-methoxy-3-(4-methyl-pyridin-2-yl)-1,3,4,6,7,11b-hexahydro-2H-pyrido[2,1a]isoquinolin-9-yloxy]-acetamide hydrochloride,
(2S,3S,11bS)- and (2R,3R,11bR)-2-amino-3-(4-fluoromethyl-pyridin-2-yl)-10-methoxy-1,3,4,6,7,11b-hexahydro-2H-pyrido[2,1-a]isoquinolin-9-yloxy]-acetic acid methyl ester, rac-(2S,3S,11bS)-2-amino-9-methoxy-3-m-tolyl-1,3,4,6,7,11b-hexahydro-2H-pyrido[2,1-a]isoquinolin-8-ol,
rac-2-(2-amino-9-methoxy-3-m-tolyl-1,3,4,6,7,11b-hexahydro-2H-pyrido[2,1-a]isoquinolin-8-yloxy)-acetamide, (2S,3S,11bS) and (2R,3S,11bS) diasteromers,
rac-2-( (2S,3S,11bS)-2-amino-9-methoxy-3-m-tolyl-1,3,4,6,7,11b-hexahydro-2H-pyrido[2,1-a]isoquinolin-8-yloxy)-N,N-dimethyl-acetamide,
rac-9-methoxy-8-(2-methoxy-ethoxy)-3-m-tolyl-1,3,4,6,7,11b-hexahydro-2H-pyrido[2,1-a]isoquinolin-2-ylamine, (2S,3S,11bS) and (2R,3S,11bS) diasteromers,
rac-2-(2-amino-9-methoxy-3-m-tolyl-1,3,4,6,7,11b-hexahydro-2H-pyrido[2,1-a]isoquinolin-8-yloxy)-ethanol, (2S,3S,11bS) and (2R,3S,11bS) diasteromers,
and pharmaceutically acceptable salts thereof.

21. The compound according to claim 1, selected from the group consisting of:
(2S,3S,11bS)- and (2R,3R,11bR)-3-(2,5-dimethyl-phenyl)-10-methoxy-9-[2-(1H-tetrazol-5-yl)-ethoxy]-1,3,4,6,7,11b-hexahydro-2H-pyrido[2,1-a]isoquinolin-2-ylamine hydrochloride,
(2S,3S,11bS)- and (2R,3R,11bR)-[2-amino-3-(2,5-dimethyl-phenyl)-10-methoxy-1,3,4,6,7,11b-hexahydro-2H-pyrido[2,1-a]isoquinolin-9-yloxy]-acetic acid hydrochloride,
(2S,3S,11bS)- and (2R,3R,11bR)-2-[2-amino-3-(2,5-dimethyl-phenyl)-10-methoxy-1,3,4,6,7,11b-hexahydro-2H-pyrido[2,1-a]isoquinolin-9-yloxy]-N-(1H-tetrazol-5-yl)-acetamide hydrochloride,
(2S,3S,11bS)- and (2R,3R,11bR)-2-[2-amino-3-(2,5-dimethyl-phenyl)-10-methoxy-1,3,4,6,7,11b-hexahydro-2H-pyrido[2,1-a]isoquinolin-9-yloxy]-acetamide hydrochloride,
(2S,3S,11bS)- and (2R,3R,11bR)-2-[2-amino-3-(2,5-dimethyl-phenyl)-10-methoxy-1,3,4,6,7,11b-hexahydro-2H-pyrido[2,1-a]isoquinolin-9-yloxy]-N-methyl-acetamide hydrochloride,
(2S,3S,11bS)- and (2R,3R,11bR)-N-[2-amino-10-methoxy-3-(4-methyl-pyridin-2-yl)-1,3,4,6,7,11b-hexahydro-2H-pyrido[2,1-a]isoquinolin-9-yl]-2-(1H-tetrazol-5-yl)-acetamide hydrochloride,
(2S,3S,11bS)- and (2R,3R,11bR)-[2-amino-10-methoxy-3-(4-methyl-pyridin-2-yl)-1,3,4,6,7,11b-hexahydro-2H-pyrido[2,1-a]isoquinolin-9-yloxy]-acetic acid hydrochloride,
(2S,3S,11bS)- and (2R,3R,11bR)-2-[2-amino-10-methoxy-3-(4-methyl-pyridin-2-yl)-1,3,4,6,7,11b-hexahydro-2H-pyrido[2,1-a]isoquinolin-9-yloxy]-N-(2H-tetrazol-5-yl)-acetamide hydrochloride,
(2S,3S,11bS)- and (2R,3R,11bR)-methanesulfonic acid 2-amino-10-methoxy-3-phenyl-1,3,4,6,7,11b-hexahydro-2H-pyrido[2,1-a]isoquinolin-9-yl ester,
(2S,3S,11bS)- and (2R,3R,11bR)-2-amino-10-methoxy-3-m-tolyl-1,3,4,6,7,11b-hexahydro-2H-pyrido[2,1-a]isoquinolin-9-ol,
(2S,3S,11bS)- and (2R,3R,11bR)-2-(2-amino-10-methoxy-3-m-tolyl-1,3,4,6,7,11b-hexahydro-2H-pyrido[2,1-a]isoquinolin-9-yloxy)-ethanol,
(2S,3S,11bS)- and (2R,3R,11bR)-2-(2-amino-10-methoxy-3-m-tolyl-1,3,4,6,7,11b-hexahydro-2H-pyrido[2,1-a]isoquinolin-9-yloxy)-acetamide hydrochloride,
(2S,3S,11bS) and (2R,3R,11bR)-2-amino-10-methoxy-3-(4-methyl-pyridin-2-yl)-1,3,4,6,7,11b-hexahydro-2H-pyrido[2,1-a) isoquinolin-9-ol,
(2S,3S,11bS)- and (2R,3R,11bR)-2-[2-amino-10-methoxy-3-(4-methyl-pyridin-2-yl)-1,3,4,6,7,11b-hexahydro-2H-pyrido[2,1-a]isoquinolin-9-yloxy]-ethanol,
rac-2-((2S,3S,11bS)-2-amino-9-methoxy-3-m-tolyl-1,3,4,6,7,11b-hexahydro-2H-pyrido[2,1-a]isoquinolin-8-yloxy)-N,N-dimethyl-acetamide,
and pharmaceutically acceptable salts thereof.

22. A process for the manufacture of the compound according to claim 1, which process comprises the steps of:
a) converting a compound of the formula 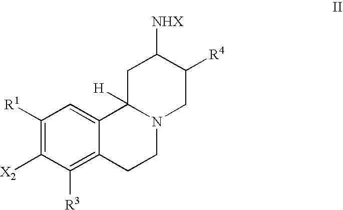
wherein X is hydrogen or tert-butoxycarbonyl, X₂ is —OH or —NH₂, R¹ and R⁴ are as defined in claim 1 and R³ is hydrogen, by side chain transformation into a compound of the formula 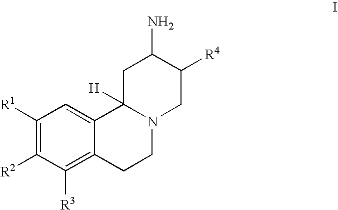
wherein R¹, R² and R⁴ are defined as in claim 1 and R³ is hydrogen, or alternatively,
b) converting a compound of the formula 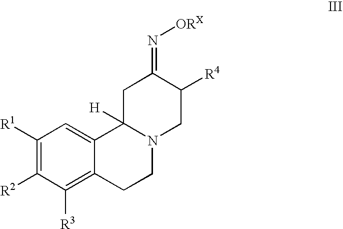
wherein R^{x} is hydrogen or benzyl and R¹ to R⁴ are as defined in claim 1,
by catalytic hydrogen reduction into a compound of the formula 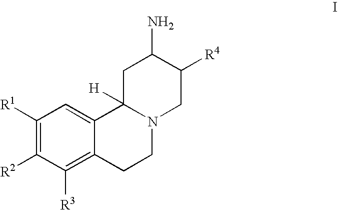
wherein R¹ to R⁴ are defined as in claim 1,
and optionally converting the compound of formula I into a pharmaceutically acceptable salt.

23. A pharmaceutical composition, comprising a therapeutically effective amount of a compound according to claim 1 and a pharmaceutically acceptable carrier and/or adjuvant.

24. A method for the treatment and/or prophylaxis of diseases which are associated with DPP-IV comprising the step of administering a therapeutically effective amount of a compound according to claim 1 to a human being or animal in need thereof.

25. The method according to claim 24, wherein said disease is diabetes, non-insulin dependent diabetes mellitus, impaired glucose tolerance, inflammatory bowel disease, encephalitis periaxialis scleroticans, rheumatoid arthritis, Colitis Ulcerosa, Morbus Crohn, psoriasis, lichen planus, benign prostate hypertrophy, hypertension, diseases wherein a diuretic agent has a beneficial effect, obesity, and/or metabolic syndrome or β-cell protection.
